(19) United States
(12) Patent Application Publication
(10) Pub. No.: US 2026/0337341 A1
(43) Pub. Date: Oct. 29, 2026

(54) NON-IONIC SURFACTANTS AND METHODS OF MAKING AND USING THE SAME

(71) Applicant: Life Technologies Corporation, Carlsbad, CA (US)

(72) Inventors: Premchendar Nandhikonda, Rockford, IL (US); Schuyler B. Corry, Burlington, NC (US); Penny J. Jensen, Marengo, IL (US); Eron Saxon, Rockford, IL (US); Daniel A. Webb, Roscoe, IL (US)

(73) Assignee: Life Technologies Corporation, Carlsbad, CA (US)

(21) Appl. No.: 19/429,699

(22) Filed: Dec. 22, 2025

Related U.S. Application Data
(60) Provisional application No. 63/787,487, filed on Apr. 11, 2025.

(51) Int. Cl. C08G 65/48

(52) U.S. Cl. CPC C08G 65/48

(57) ABSTRACT

Disclosed herein are aspects of a compound according to Formula I or Formula V

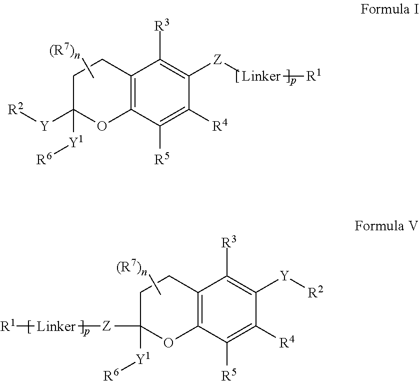

The compounds are useful as surfactants and can be used in applications for which a surfactant is indicated. The disclosed compounds are biodegradable and do not form toxic by-products when degraded. As such, the disclosed compounds can be used as replacements for current surfactants, such as current octylphenol ethoxylates (OPEs) and nonylphenol ethoxylates (NPEs) that have toxic effects when degraded. Also disclosed are aspects of a method for making the compounds and methods for using the compounds in cell assays.

### CROSS REFERENCE TO RELATED APPLICATIONS

This application claims the benefit of priority under 35 U.S.C. § 119(e) to U.S. Provisional Application No. 63/787,487 filed on Apr. 11, 2025, the contents of which are hereby expressly incorporated herein by reference in their entirety as though fully set forth herein.

### FIELD

The present disclosure is directed to new biodegradable surfactants and methods of making and using the same.

### BACKGROUND

Currently available non-ionic surfactants, such as Triton™ X-100 and Tergitol™ NP-40, are part of a group of chemicals known as octylphenol ethoxylates (OPEs) and nonylphenol (NPEs) that, when broken down in the environment, have toxic effects on aquatic organisms at extremely low concentrations. OPEs and NPEs are toxic xenobiotic compounds classified as endocrine disrupters capable of interfering with the hormonal system of numerous organisms. The use of OPEs and NPEs has already been banned for most uses in the EEA, UK, and Switzerland requiring capture, diversion, and incineration of all waste with limited exemptions or authorization.

Many other countries are evaluating similar actions where exemptions or authorizations are less clear than in the EU. There exists a need in the art for new surfactants that are biodegradable and do not exhibit the toxic effects associated with currently available non-ionic surfactants.

### SUMMARY

Disclosed herein are aspects of a compound having a structure according to Formula I.

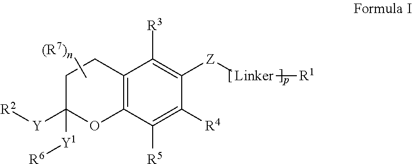

With respect to Formula I, R¹ is a hydrophilic group and R² is a lipophilic group. Each or R³, R⁴ and R⁵ independently is H, C₁₋₆alkyl, —OC₁₋₆alkyl, C₃₋₆cycloalkyl, or —OC₃₋₆cycloalkyl, R⁶ is H or C₁₋₃₀alkyl, R⁷ is C₁₋₆alkyl, and n is 0, 1, 2, 3, or 4. Y is a bond, O, or N(R^{a}), and Y¹ is a bond, O, or N(R^{a}). Z is O, N(R^{a}), C(═O), (C═O)N(R^{a}), C(═O)O, N(R^{a})C(═O), OC(═O), or a bond, Linker is —(CH₂)ₓO—, —(CH₂)ₓC(═O)O—, —(CH₂)ₓC(═O)N—, —(CH₂)ₓOC(═O)—, or —(CH₂)ₓNC(═O)—, where x is 1, 2, 3, or 4, and each R^{a} independently is H, C₁₋₄alkyl. And p is 0 or 1.

Also disclosed are aspects of a compound having a structure according to Formula V.

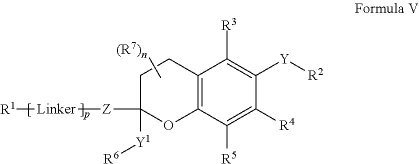

With respect to Formula V, R¹, R², R³, R⁴, R⁵, R⁶, R⁷, Y, Y¹, Z, Linker, n and p are as previously defined for Formula I.

Also disclosed herein are aspects of a method, comprising exposing a sample to a compound disclosed herein. And aspects of a method of lysing a cell also are disclosed. The method may comprise contacting the cell with an amount of a compound disclosed herein sufficient to facilitate cell lysis.

Further disclosed aspects concern a composition and/or a kit comprising a compound disclosed herein. In some aspects, the kit may further comprise a container.

The foregoing and other objects, features, and advantages of the disclosure will become more apparent from the following detailed description, which proceeds with reference to the accompanying figures.

### BRIEF DESCRIPTION OF THE DRAWINGS

FIGS. 1A and 1B are graphs of percentage of the control versus surfactant, illustrating the percentage of protein recovery with each surfactant (FIG. 1A) and the background signal of the surfactant only (FIG. 1).

FIG. 2 provides digital images of the lysates produced by different concentrations of surfactant, illustrating the presence of the nuclei.

FIG. 3 is an SDS-page gel stained with Coomassie staining, illustrating the Coomassie bands of the various surfactants at different concentrations.

FIG. 4 is a western blot, illustrating the HSP70 and HisH3 bands for the various surfactants at different concentrations.

FIG. 5 is a western blot, illustrating the EGFR and Cav1 bands for the various surfactants at different concentrations.

FIGS. 6A and 6B are graphs of percentage of the control versus surfactant, illustrating the percentage of protein recovery with each surfactant (FIG. 6A) and the background signal of the surfactant only (FIG. 6B).

FIG. 7 provides digital images of the lysates produced by different concentrations of surfactant, illustrating the presence of the nuclei.

FIG. 8 is an SDS-page gel stained with Coomassie staining, illustrating the Coomassie bands of the various surfactants at different concentrations.

FIG. 9 is a western blot, illustrating the EGFR and Cav1 bands for the various surfactants at different concentrations.

FIG. 10 is a western blot, illustrating the ATP1A1 and COXIV bands for the various surfactants at different concentrations.

FIGS. 11A and 11B are graphs of percentage of the control versus surfactant, illustrating the percentage of protein recovery with each surfactant (FIG. 11A) and the background signal of the surfactant only (FIG. 11B).

FIG. 12 provides digital images of the lysates produced by different concentrations of surfactant, illustrating the presence of the nuclei.

FIG. 13 is an SDS-page gel stained with Coomassie staining, illustrating the Coomassie bands of the various surfactants at different concentrations.

FIG. 14 is a western blot, illustrating the EGFR and Cav1 bands for the various surfactants at different concentrations.

FIG. 15 is a western blot, illustrating the ATP1A1 and COXIV bands for the various surfactants at different concentrations.

### DETAILED DESCRIPTION

### I. Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” or “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods and in some aspects encompasses a range up to ±15% of that numerical value, unless the context clearly dictates otherwise.

Certain functional group terms used herein include a symbol “-”, which is used to show how the defined functional group attaches to, or within, the compound to which it is bound. Also, a dashed bond (i.e., “ [symbol] ”) as used in certain formulas described herein indicates an “optional” bond to a substituent or atom of the formula other than hydrogen in the sense that the bond (and in some aspects, the substituent) may or may not be present. In any formulas comprising a dashed bond, if the optional bond and/or any corresponding substituent is not present, then the valency requirements of any atom(s) bound thereto is completed by a bond to a hydrogen atom.

The symbol “ [symbol] ” is used to indicate a bond disconnection in abbreviated structures/formulas provided herein. A person of ordinary skill in the art recognizes that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

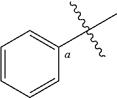 [SMILES: *c1ccccc1]

comprises a hydrogen atom attached to each carbon atom of the phenyl ring other than the “a” carbon, even though such hydrogen atoms are not illustrated.

If a group R is depicted as “floating” on a ring system, as for example in the group:

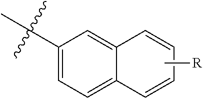

then, unless otherwise defined, a substituent R can reside on any atom of the fused bicyclic ring system, excluding the atom carrying the bond with the “ [symbol] ” symbol, so long as a stable structure is formed. In the example depicted, the R group can reside on an atom in either the left side or the right side ring of the naphthylene ring system.

When there are more than one such depicted “floating” groups, as for example in the formula:

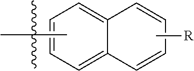

where there are two groups, namely, the R and the bond indicating attachment to a parent structure; then, unless otherwise defined, the “floating” groups can reside on any atoms of the ring system, again assuming each replaces a depicted, implied, or expressly defined hydrogen on the ring system and a chemically stable compound would be formed by such an arrangement.

Any functional group disclosed herein and/or defined above can be substituted or unsubstituted, unless otherwise indicated herein.

To facilitate review of the disclosure, the following explanations of specific terms are provided.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well. Aliphatic groups may be substituted with one or more groups other than hydrogen, such as aliphatic, heteroaliphatic, haloaliphatic, haloheteroaliphatic, aromatic, or an organic functional group.

Alkenyl: An unsaturated monovalent hydrocarbon having at least two carbon atoms to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g. cycloalkenyl), cis, or trans (e.g., E or Z).

Alkoxy: —O-aliphatic, such as —O-alkyl, —O-alkenyl, —O-alkynyl; with exemplary aspects including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds). Alkoxy groups may be substituted with one or more groups other than hydrogen, such as aliphatic, heteroaliphatic, haloaliphatic, haloheteroaliphatic, aromatic, or an organic functional group.

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (as in cycloalkyl).

Alkynyl: An unsaturated monovalent hydrocarbon having at least two carbon atoms to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (as in cycloalkynyl).

Amino: —NR^{b}R^{c}, wherein each of R^{b} and R^{c} independently is selected from hydrogen, aliphatic, heteroaliphatic, haloaliphatic, haloheteroaliphatic, aromatic, or an organic functional group, and can be substituted with one or more groups other than hydrogen, such as aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

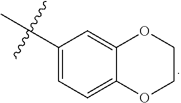 [SMILES: *c1ccc2c(c1)OCCO2]

However, in certain examples, context or express disclosure may indicate that the point of attachment is through a non-aromatic portion of the condensed ring system. For example,

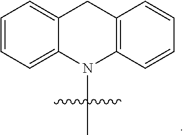 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aroxy: —O-aromatic. Aroxy groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅₋₁₅), such as five to ten carbon atoms (C₅₋₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as an aliphatic, heteroaliphatic, haloaliphatic, haloheteroaliphatic, aromatic, or an organic functional group.

Azo: —N═NR^{a} wherein R^{a} is hydrogen, aliphatic, heteroaliphatic, haloaliphatic, haloheteroaliphatic, aromatic, or an organic functional group. Azo groups may be substituted with one or more groups other than hydrogen, such as aliphatic, heteroaliphatic, haloaliphatic, haloheteroaliphatic, aromatic, or an organic functional group.

Carbamate: —OC(O)NR^{b}R^{c}, wherein each of R^{b} and R^{c} independently is selected from hydrogen, aliphatic, heteroaliphatic, haloaliphatic, haloheteroaliphatic, aromatic, or an organic functional group. Carbamate groups can be substituted with one or more groups other than hydrogen, such as aliphatic, heteroaliphatic, haloaliphatic, haloheteroaliphatic, aromatic, or an organic functional group.

Carbonate: —OC(O)OR^{a}, wherein R^{a} is selected from aliphatic, heteroaliphatic, haloaliphatic, haloheteroaliphatic, aromatic, or an organic functional group. Carbonate groups can be substituted with one or more groups other than hydrogen, such as aliphatic, heteroaliphatic, haloaliphatic, haloheteroaliphatic, aromatic, or an organic functional group. In independent aspects, R^{a} can be hydrogen.

Carboxyl: —C(O)OH.

Carboxylate: —C(O)O— or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Cyano: —CN.

Degree of Polymerization: The number of monomer units in a polymer. In the context of the present disclosure, when discussing, for example, polyalkene oxide and/or polyalkylene amine polymers comprising repeat units of monomers, the degree of polymerization is typically defined by a mass average molecular weight.

Disulfide: —SSR^{a}, wherein R^{a} is selected from hydrogen, aliphatic, heteroaliphatic, haloaliphatic, haloheteroaliphatic, aromatic, or an organic functional group. Disulfide groups can be substituted with one or more groups other than hydrogen, such as aliphatic, heteroaliphatic, haloaliphatic, haloheteroaliphatic, aromatic, or an organic functional group.

Dithiocarboxylic: —C(S)SR^{a} wherein R^{a} is selected from hydrogen, aliphatic, heteroaliphatic, haloaliphatic, haloheteroaliphatic, aromatic, or an organic functional group.

Dithiocarboxylic groups can be substituted with one or more groups other than hydrogen, such as aliphatic, heteroaliphatic, haloaliphatic, haloheteroaliphatic, aromatic, or an organic functional group.

EcoSurf™ EH-9: EcoSurf™ EH-9 (EcoSurf™-EH9, EH9) is a biodegradable nonionic surfactant comprising ethylene oxide and propylene oxide polymer units. Synonyms include: 2-Ethyl hexanol EoPo copolymer (9 EO), Ethyl hexanol ethoxylated propoxylated, and Ethylene oxide-propylene oxide copolymer mono(2-ethylhexyl) ether.

Ester: —C(O)OR^{a} or —OC(O)R^{a}, wherein R^{a} is selected from aliphatic, heteroaliphatic, haloaliphatic, haloheteroaliphatic, aromatic, or an organic functional group. Ester groups can be substituted with one or more groups other than hydrogen, such as aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic, including any polymers thereof having repeats of any such groups (e.g., polyalkene oxide compounds). Ether groups can be substituted with one or more groups other than hydrogen, such as aliphatic, heteroaliphatic, haloaliphatic, haloheteroaliphatic, aromatic, or an organic functional group.

Halo (or halide or halogen): Fluoro, chloro, bromo, or iodo. In some aspects, halo can also include astatine.

Haloaliphatic: An aliphatic group wherein one or more hydrogen atoms, such as one to 10 hydrogen atoms, independently is replaced with a halogen atom, such as fluoro, bromo, chloro, or iodo. Haloaliphatic groups can be substituted with one or more groups other than hydrogen, such as aliphatic, heteroaliphatic, haloaliphatic, haloheteroaliphatic, aromatic, or an organic functional group.

Haloheteroaliphatic: A heteroaliphatic group wherein one or more hydrogen atoms, such as one to 10 hydrogen atoms, independently is replaced with a halogen atom, such as fluoro, bromo, chloro, or iodo. Haloheteroaliphatic groups can be substituted with one or more groups other than hydrogen, such as aliphatic, heteroaliphatic, haloaliphatic, haloheteroaliphatic, aromatic, or an organic functional group.

Heteroatom: An atom other than carbon or hydrogen, such as (but not limited to) oxygen, nitrogen, sulfur, silicon, boron, selenium, or phosphorous. In particular disclosed aspects, such as when valency constraints do not permit, a heteroatom does not include a halogen atom.

Heteroaliphatic: An aliphatic group comprising at least one carbon atom and from one heteroatom to 20 heteroatoms, such as one to 15 heteroatoms, or one to 5 heteroatoms, which can be selected from, but not limited to, oxygen, nitrogen, sulfur, silicon, boron, selenium, phosphorous, and oxidized forms thereof within the group. Alkoxy, ether, PEG (polyethylene glycol), and thioether groups are exemplary (but non-limiting) examples of heteroaliphatic. Heteroaliphatic groups can be substituted with one or more groups other than hydrogen, such as aliphatic, heteroaliphatic, haloaliphatic, haloheteroaliphatic, aromatic, or an organic functional group.

Heterocycloaliphatic: A cyclic heteroaliphatic (i.e., non-aromatic) group of from 3-25 ring atoms, such as from 3 to 20 ring atoms, from 3 to 10 ring atoms, from 3-8 ring atoms or from 3-6 ring atoms. A heterocycloaliphatic includes at least one ring carbon atom and at least one ring heteroatom as defined herein. A heterocycloaliphatic group may be a heterocycloalkyl, heterocycloalkenyl, or heterocycloalkynyl group. In some aspects, the at least one ring heteroatom is selected from oxygen, nitrogen, sulfur, or a combination thereof. Heterocycloaliphatic groups can be substituted with one or more groups other than hydrogen, such as aliphatic, heteroaliphatic, haloaliphatic, haloheteroaliphatic, aromatic, or an organic functional group. Example heterocycloaliphatic groups include, but are not limited to, tetrahydropyranyl, tetrahydrofuryl, pyranyl, oxiranyl, azetidinyl, dioxolanyl, piperidinyl, piperazinyl, morphinyl, and the like.

Hydrophilic: A hydrophilic group according to the present disclosure is a functional group or other chemical group that exhibits affinity for water.

Hydrophilic-Lipophilic Balance (HLB): A numerical value that represents the balance of the size and strength of the hydrophilic and lipophilic moieties of a surfactant compound. The HLB scale ranges from 0 to 20.

Hydroxyl: —OH

Ketone: —C(O)R^{a}, wherein R^{a} is selected from aliphatic, heteroaliphatic, haloaliphatic, haloheteroaliphatic, aromatic, or an organic functional group. Ketone groups can be substituted with one or more groups other than hydrogen, such as aliphatic, heteroaliphatic, haloaliphatic, haloheteroaliphatic, aromatic, or an organic functional group.

Lipophilic: A lipophilic group according to the present disclosure is a functional group or other chemical group that lacks affinity for water.

Tergitol™ NP-40: A compound having a structure

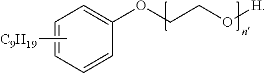

Organic Functional Group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic; phosphonate; phosphate; silyl ether; sulfinyl; sulfonamide; thial; or combinations thereof. Organic functional groups can be substituted with one or more groups other than hydrogen, such as aliphatic, heteroaliphatic, haloaliphatic, haloheteroaliphatic, aromatic, or an organic functional group.

Oxime: —CR^{a}═NOH, wherein R^{a} is hydrogen, aliphatic, heteroaliphatic, haloaliphatic, haloheteroaliphatic, aromatic, or an organic functional group. Oxime groups can be substituted with one or more groups other than hydrogen, such as aliphatic, heteroaliphatic, haloaliphatic, haloheteroaliphatic, aromatic, or an organic functional group.

Peroxy: —O—OR^{a} wherein R^{a} is hydrogen, aliphatic, heteroaliphatic, haloaliphatic, haloheteroaliphatic, aromatic, or an organic functional group. Peroxy groups can be substituted with one or more groups other than hydrogen, such as aliphatic, heteroaliphatic, haloaliphatic, haloheteroaliphatic, aromatic, or an organic functional group.

Phosphate: —O—P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. The R^{a} groups of the phosphate can be substituted with one or more groups other than hydrogen, such as aliphatic, heteroaliphatic, haloaliphatic, haloheteroaliphatic, aromatic, or an organic functional group.

Phosphonate: —P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. The R^{a} groups of the phosphonate group can be substituted with one or more groups other than hydrogen, such as aliphatic, heteroaliphatic, haloaliphatic, haloheteroaliphatic, aromatic, or an organic functional group.

Silyl Ether: —OSiR^{a}R^{b}R^{c}, wherein each of R^{a}, R^{b} and R^{c} independently is selected from hydrogen, aliphatic, heteroaliphatic, haloaliphatic, haloheteroaliphatic, aromatic, or an organic functional group. Silyl ether groups can be substituted with one or more groups other than hydrogen, such as aliphatic, heteroaliphatic, haloaliphatic, haloheteroaliphatic, aromatic, or an organic functional group.

Sulfinyl: —S(O)R^{a}, wherein R^{a} is selected from hydrogen, aliphatic, heteroaliphatic, haloaliphatic, haloheteroaliphatic, aromatic, or an organic functional group. Sulfinyl groups can be substituted with one or more groups other than hydrogen, such as aliphatic, heteroaliphatic, haloaliphatic, haloheteroaliphatic, aromatic, or an organic functional group.

Sulfonyl: —SO₂R^{a}, wherein R^{a} is selected from hydrogen, aliphatic, heteroaliphatic, haloaliphatic, haloheteroaliphatic, aromatic, or an organic functional group. Sulfonyl groups can be substituted with one or more groups other than hydrogen, such as aliphatic, heteroaliphatic, haloaliphatic, haloheteroaliphatic, aromatic, or an organic functional group.

Sulfonamide: —SO₂NR^{b}R^{c} or —N(R^{b})SO₂R^{c}, wherein each of R^{b} and R^{c} independently is selected from hydrogen, aliphatic, heteroaliphatic, haloaliphatic, haloheteroaliphatic, aromatic, or an organic functional group. Sulfonamide groups can be substituted with one or more groups other than hydrogen, such as aliphatic, heteroaliphatic, haloaliphatic, haloheteroaliphatic, aromatic, or an organic functional group.

Sulfonate: —SO₃⁻, wherein the negative charge of the sulfonate group may be balanced with an M⁺ counter ion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Surfactant: A compound that lowers surface tension between two fluids (liquids and/or gases) and/or between a solid and a fluid. Surfactants can exhibit properties the allow their use as surfactants, dispersants, emulsifiers, foaming agents, wetting agents, lubricants, or a combination thereof.

Tocopherol: A class of vitamin E compounds. One example compound is alpha-tocopherol which has the structure

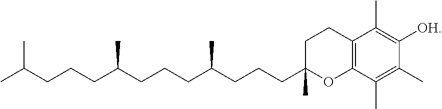 [SMILES: Cc1c(C)c2c(c(C)c1O)CC[C@@](C)(CCC[C@H](C)CCC[C@H](C)CCCC(C)C)O2]

Terminating Group: A functional group that is used to terminate an R¹ group of the formulas according to the present disclosure.

Thial: —C(S)H.

Thiocarboxylic acid: —C(O)SH, or —C(S)OH.

Thiocyanate: —S—CN or —N═C═S.

Thioester: —C(O)SR^{a} or —C(S)OR^{a} wherein R^{a} is selected from hydrogen, aliphatic, heteroaliphatic, haloaliphatic, haloheteroaliphatic, aromatic, or an organic functional group. Thioester groups can be substituted with one or more groups other than hydrogen, such as aliphatic, heteroaliphatic, haloaliphatic, haloheteroaliphatic, aromatic, or an organic functional group.

Thioether: —S-aliphatic or —S-aromatic, such as —S-alkyl, —S-alkenyl, —S-alkynyl, —S-aryl, or —S-heteroaryl; or -aliphatic-S-aliphatic, -aliphatic-S-aromatic, -aromatic-S-aliphatic, or -aromatic-S-aromatic. Thioether groups can be substituted with one or more groups other than hydrogen, such as aliphatic, heteroaliphatic, haloaliphatic, haloheteroaliphatic, aromatic, or an organic functional group.

Thioketone: —C(S)R^{a} wherein R^{a} is selected from hydrogen, aliphatic, heteroaliphatic, haloaliphatic, haloheteroaliphatic, aromatic, or an organic functional group. Thioketone groups can be substituted with one or more groups other than hydrogen, such as aliphatic, heteroaliphatic, haloaliphatic, haloheteroaliphatic, aromatic, or an organic functional group.

Triton™ X-100: A compound having a structure

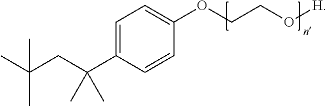

Trolox: A compound having a structure

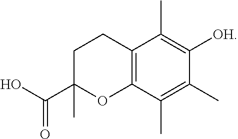 [SMILES: Cc1c(C)c2c(c(C)c1O)CCC(C)(C(=O)O)O2]

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diasteromers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms. In cases of limited rotation, e.g. around the amide bond or between two directly attached rings such as the pyrazolyl and pyridinyl rings, atropisomers are also possible and are also specifically included in the compounds of the invention.

### II. Introduction

Surfactants play an important role in life science technology and other various industries. With respect to their roles in the life sciences, they are often important components used in both nucleic acid purification and direct PCR processes. For example, in direct PCR, surfactants are needed to enhance the efficiency of the reaction by improving the accessibility of the DNA template. They also can be used to aid in cell lysis, viral inactivation, enhancing DNA accessibility, reducing non-specific binding, and ultimately contributing to the success of obtaining pure nucleic acids and amplifying specific DNA targets. Other processes and/or applications that rely on surfactants are discussed herein and can include, but are not limited to, DNA hybridization, kits for diagnosing allergy-, asthma-, and autoimmune-related diseases, nucleic acid-based sample preparation workflows, and the like.

Current non-ionic surfactants, such as Triton™ X-100 and/or Tergitol™ NP-40, that are available as surfactants for myriad biological and/or other industrial uses have a trade-off associated with good performance: they produce degradation by-products that exhibit endocrine disruption effects that interfere with the hormonal system of numerous organisms, particularly aquatic species. These surfactants are often used in sample preparation products, including protein extraction buffers, wash buffers, protein interaction kits, and protein purification kits. To date, no alternative surfactants are available as direct replacements for Triton™ X-100 and/or Tergitol™ NP-40, particularly those that are suitable for life science applications.

While less hazardous surfactants are available, they do not meet quality or performance standards across a broad range of products or applications, including sensitive biological assays typically used in life science technologies.

The present disclosure is directed to new compounds which are surfactants. The new compounds may be used to replace current surfactants, like Triton™ X-100 and/or Tergitol™ NP-40. The disclosed compounds are less hazardous than Triton™ X-100 and/or Tergitol™ NP-40. The compounds may work as effectively as, or better than, Triton™ X-100 and/or Tergitol™ NP-40. The disclosed compounds can be prepared using cost-effective methods. The disclosed compounds are biodegradable without producing toxic by-products like the endocrine disrupting by-products that are produced from degradation of Triton™ X-100 and/or Tergitol™ NP-40. The disclosed compounds are suitable for use as surfactants in myriad applications/industries.

### III. Compounds

Disclosed herein are aspects of a compound that exhibits surfactant properties. In some aspects, the compound has a structure according to Formula I.

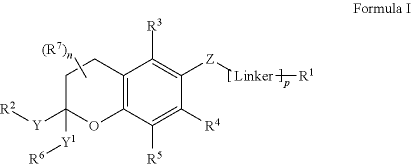

With respect to Formula I, R¹ is a hydrophilic group and R² is a lipophilic group.

Each of R³, R⁴ and R⁵ independently is H, C₁₋₆alkyl, —OC₁₋₆alkyl, C₃₋₆cycloalkyl, or —OC₃₋₆cycloalkyl. In some aspects, each of R³, R⁴ and R⁵ independently is H or C₁₋₆alkyl.

In some aspects, all of R³, R⁴ and R⁵ are H. In other aspects, at least one of R³, R⁴ and R⁵ is not H. In some aspects, one of R³, R⁴ and R⁵ is not H. In other aspects, two of R³, R⁴ and R⁵ are not H. And in certain aspects, all of R³, R⁴ and R⁵ are not H. In particular aspects, all of R³, R⁴ and R⁵ are C₁₋₆alkyl, such as methyl.

In some aspects, R³, R⁴, and R⁵ are independently —O—C₁₋₆alkyl, —O—C₃₋₆cycloalkyl,

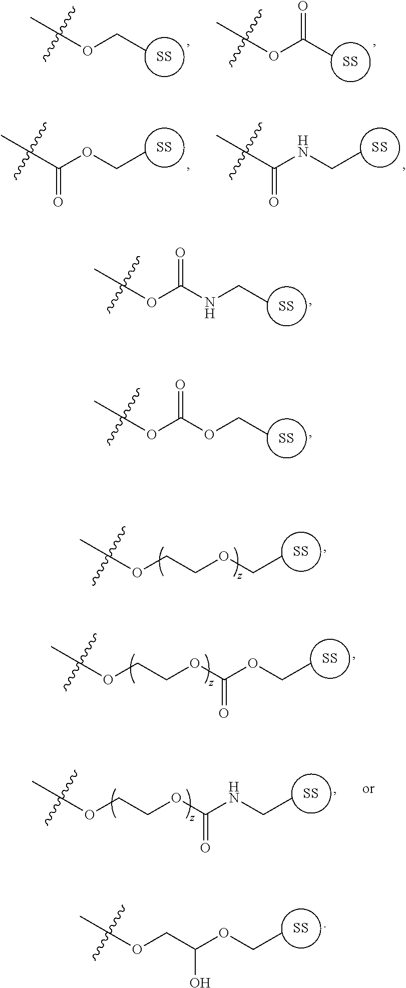

In some aspects, the solid support (SS) may be selected from agarose resins such as Sepharose, polystyrene resins such as Merrifield resin, Wang resin, and Rink amide resin, polystyrene beads, PEG-based beads such as TentaGel and pegylated-polystyrene beads, silica and silica-gel supports including functionalized silica beads, alumina supports, magnetic silica or polymer-coated magnetic beads, controlled-pore glass beads, functionalized magnetic beads, functionalized silica gel, cellulose and modified cellulose resins, chitosan beads, dextran-based supports such as Sephadex, and pre-functionalized systems including NHS-activated beads, controlled-pore glass beads, maleimide-activated beads, epoxy-activated supports, and streptavidin-biotin capture systems, maleimide-activated beads, and epoxy-activated supports.

In some aspects, the solid support (SS) may be a resin, a bead, a particle, magnetic bead, a non-magnetic bead, an ion exchange matrix, an affinity chromatography matrix, a size exclusion chromatography matrix, a hydrophobic interaction chromatography matrix, an immobilized metal affinity chromatography, a reverse phase chromatography matrix, immunoaffinity chromatography matrix, or a mixed mode chromatography matrix, or any of the solid supports disclosed herein.

In some aspects, R³, R⁴, and R⁵ are independently —O—C₁₋₆alkyl, —O—C₃₋₆cycloalkyl,

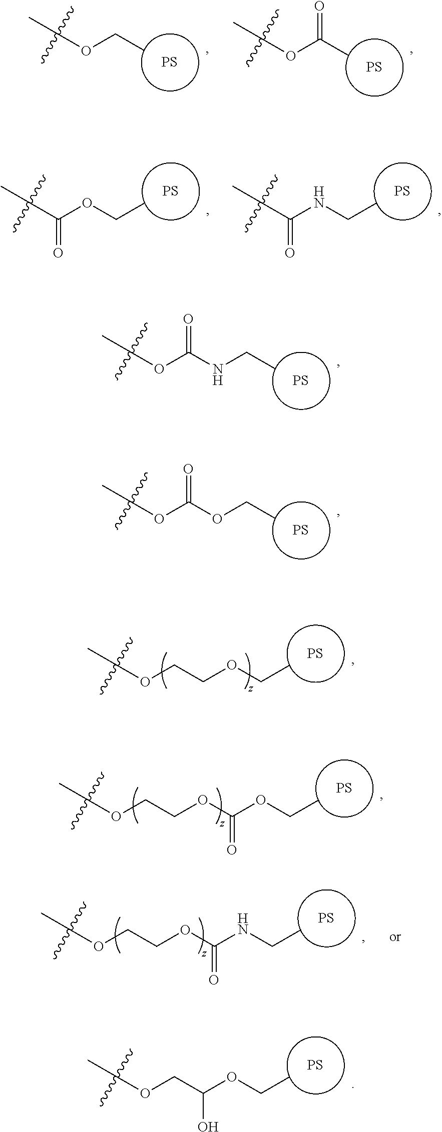

R⁶ is H or C₁₋₃₀alkyl, such as C₁₋₂₅alkyl, C₁₋₂₀alkyl, C₁₋₁₅alkyl, C₁₋₁₀alkyl, or C₁₋₆alkyl. In some aspects, R⁶ is H or C₁₋₄alkyl. In certain aspects, R⁶ is H or methyl. In some particular aspects, R⁶ is methyl, but in other particular aspects, R⁶ is H.

R⁷ is C₁₋₆alkyl, and n is 0, 1, 2, 3, or 4, such as 0, 1 or 2. In some aspects, n is 0.

Y is a bond, O, or N(R^{a}). In some aspects, Y is a bond.

Y¹ is a bond, O, or N(R^{a}). In some aspects, Y¹ is a bond. In certain aspects, Y¹ is a bond and R⁶ is H, and in certain other aspects, Y¹ is a bond and R⁶ is methyl.

Z is O, N(R^{a}), C(═O), (C═O)N(R^{a}), C(═O)O, N(R^{a})C(═O), OC(═O), or a bond. In certain aspects, Z is O. In other aspects, Z is a bond.

Linker is —(CH₂)ₓO—, —(CH₂)ₓC(═O)O—, —(CH₂)ₓC(═O)N—, —(CH₂)ₓOC(═O)—, or —(CH₂)ₓNC(═O)—.

x is 1, 2, 3, or 4, such as 1, 2, or 3, and in some aspects, x is 2 or 3. In certain aspects, x is 2, but in other aspects, x is 3.

p is 0 or 1. In some aspects p is 0. In other aspects, p is 1. In certain aspects, p is 1, Z is —OC(═O)— and Linker is —(CH₂)ₓC(═O)O—. In particular aspects, Z and Linker together form —OC(═O)CH₂CH₂C(═O)O—.

Each R^{a} independently is H, C₁₋₄alkyl, such as H, methyl, ethyl, or isopropyl. In some aspects, each R^{a} independently is H or methyl, and in certain aspects, each R^{a} is H.

In some aspects, R¹ is R^{b} or a heterocycloaliphatic.

R^{b} is a polyalkylene oxide group, such as polyethylene glycol (PEG), polypropylene glycol (PPG), or a combination thereof, optionally terminated in an alkyl group, such as methyl. In such aspects, the PEG or PPG group may be referred to as mPEG or mPPG. In some aspects, R^{b} is —([(CH₂)_{y}]—O)_{z}—R^{c} where each y independently is 2 or 3, and z is from 4 to 25.

R^{c} is H or C₁₋₄alkyl, such as H or C₁₋₂alkyl, and in some aspects R^{c} is H or methyl.

In certain aspects, each y is 2. But in other certain aspects, each y is 3.

In some aspects, z is from 4 to 30, such as from 6 to 25, form 6 to 20, from 6 to 15, from 6 to 12, or from 8 to 12. In certain aspects, z is 8 (for example PEG8), 9 (for example PEG9), 10 (for example PEG10), 11 (for example PEG 11), or 12 (for example PEG12).

In a particular aspect, R^{b} is PEG8 optionally terminated by methyl (that is, R^{c} is H or methyl).

In a particular aspect, R^{b} is PEG9 optionally terminated by methyl (that is, R^{c} is H or methyl).

In a particular aspect, R^{b} is PEG10 optionally terminated by methyl (that is, R^{c} is H or methyl).

In a particular aspect, R^{b} is PEG11 optionally terminated by methyl (that is, R^{c} is H or methyl).

In a particular aspect, R^{b} is PEG12 optionally terminated by methyl (that is, R^{c} is H or methyl).

In some aspects, R^{b} is a PEG or PPG group having a mass average molecular weight ranging from 500 to 800 daltons, such as 525 to 800, or 550 to 800, or 575 to 800, or 600 to 800, or 625 to 800, or 650 to 800, or 675 to 800, or 700 to 800, or 725 to 800, or 750 to 800, or 775 to 800. In any aspects, the PEG or PPG group is terminated by H, methyl, or a combination thereof. In some aspects, the PEG or PPG group is terminated by H but in other aspects, the PEG or PPG group is terminated by methyl. In particular aspects, R^{b} is a PEG group having a mass average molecular weight ranging from 500 to 575, or 700 to 775. In representative aspects, the PEG group has a mass average molecular weight of 500, 550, 700, or 750. In any such aspects comprising a PEG or PPG group, the average molecular weights do not include the weight of any terminating group.

In some aspects, R¹ is R^{b}.

In other aspects, R¹ is a heterocycloaliphatic group, such as an oxygen-containing heterocycloaliphatic group, and may be a 5- or 6-membered oxygen-containing heterocycloaliphatic group. In some aspects, R¹ is a sugar moiety or is derived from a sugar moiety, such as a 6-membered sugar moiety. In some aspects, R¹ is derived from glucuronic acid, trehalose, a glucopyranoside, or a sugar moiety comprising 3 or 4 sugar moieties optionally 3-4 glucose moieties.

In some aspects, R¹ is

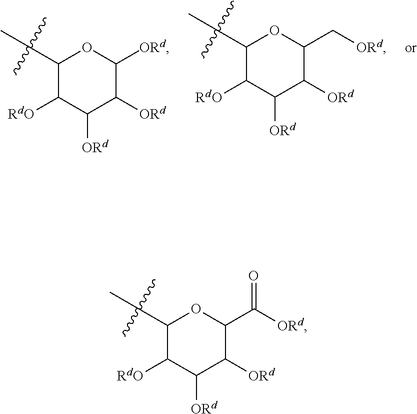

where each R^{d} independently is H, R^{b},

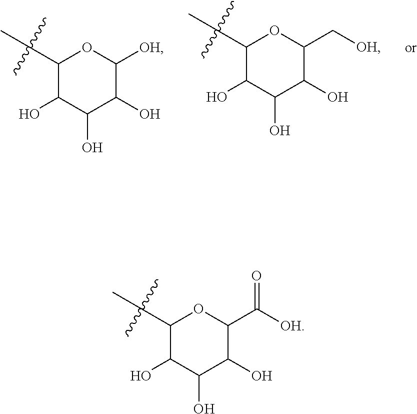 [SMILES: *C1OC(O)C(O)C(O)C1O ; *C1OC(CO)C(O)C(O)C1O ; *C1OC(C(=O)O)C(O)C(O)C1O]

In some aspects, each R^{d} is H and R¹ is

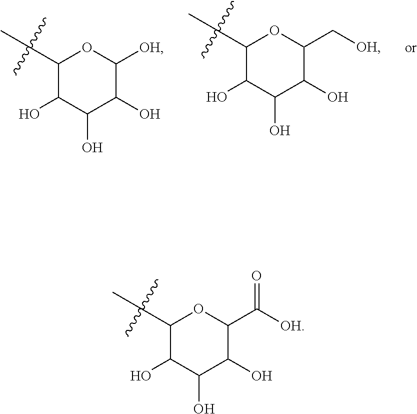 [SMILES: *C1OC(O)C(O)C(O)C1O ; *C1OC(CO)C(O)C(O)C1O ; *C1OC(C(=O)O)C(O)C(O)C1O]

In some aspects, at least one R^{d} is R^{b}. In some such aspects, any R^{d} that are not R^{b} are H.

In some aspects, one R^{d} is R^{b} and three R^{d} are H.

In some aspects, two R^{d} are R^{b} and two R^{d} are H.

In some aspects, three R^{d} are R^{b} and one R^{d} is H.

In some aspects, all four R^{d} are R^{b}.

In some aspects, R² is an aliphatic group, an aromatic group, or a combination of aliphatic and aromatic groups, such as, solely by way of example, benzyl. In some aspects, R² is an aliphatic group, such as a C₁₋₃₀alkyl, C₂₋₃₀alkenyl, or C₂₋₃₀alkynyl. R² may be a straight chain, branched chain, and/or be cyclic or contain a cyclic moiety. In some aspects, R² is C₁₋₃₀alkyl, such as C₁₋₂₅alkyl, C₁₋₂₀alkyl, C₁₋₁₅alkyl, C₁₋₁₂alkyl, or C₁₋₁₀alkyl. In other aspects, R² is C₂₋₃₀alkyl, such as C₂₋₂₅alkyl, C₂₋₂₀alkyl, C₂₋₁₅alkyl, C₂₋₁₂alkyl, or C₂₋₁₀alkyl, or C₃₋₃₀alkyl, such as C₃₋₂₅alkyl, C₃₋₂₀alkyl, C₃₋₁₅alkyl, C₃₋₁₂alkyl, or C₃₋₁₀alkyl, for example C₄₋₃₀alkyl, such as C₄₋₂₅alkyl, C₄₋₂₀alkyl, C₄₋₁₅alkyl, C₄₋₁₂alkyl, or C₄₋₁₀alkyl. In certain aspects, R² is C₃alkyl (for example, isopropyl), C₄alkyl, C₅alkyl, C₆alkyl, C₇alkyl, C₈alkyl, C₉alkyl, C₁₀alkyl, C₁₂alkyl, C₁₄alkyl, or C₁₆alkyl. In certain aspects, R² is a straight chain, but in certain other aspects, R² is branched, and may have 1, 2, 3, 4 or more branches.

In a particular aspect, R² is

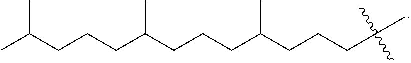 [SMILES: *CCCC(C)CCCC(C)CCCC(C)C]

In another aspect, R² is methyl and R⁶ is methyl. And in some such aspects, Y and Y¹ are both bonds.

In other aspects, R² is aromatic or a combination of aromatic and aliphatic groups. R² may be aryl, such as phenyl, or heteroaryl. And in any such aspects, R² may be substituted with 1, 2, 3, 4, or 5 lipophilic substituents, such as C₁₋₂₅alkyl, C₂₋₂₅alkenyl, or C₂₋₂₅alkynyl, or any combination thereof. Additionally, or alternatively, the aromatic moiety may be connected to the rest of the molecule by an aliphatic linker. In some such aspects, R² is —(C₁₋₁₀alkyl)-(phenyl optionally substituted with from 1-5 substituent selected from C₁₋₂₅alkyl, C₂₋₂₅alkenyl, or C₂₋₂₅alkynyl).

In some aspects, the hydrophilic and lipophilic groups for R¹ and R², respectively, are selected to provide a hydrophilic-lipophilic balance (“HLB”) value (calculated using Griffin's method) ranging from 10 to 18, such as 10 to 12, or 10 to 13, or 11 to 13, or 11 to 14, or 12 to 14, or 12 to 15, or 13 to 15, or 13 to 16, or 14 to 16, or 11 to 18, or 12 to 18, or 13 to 18, or 14 to 18, or 15 to 18, or 16 to 18, or 17 to 18.

In some aspects, the compound has a structure according to Formulas II or III.

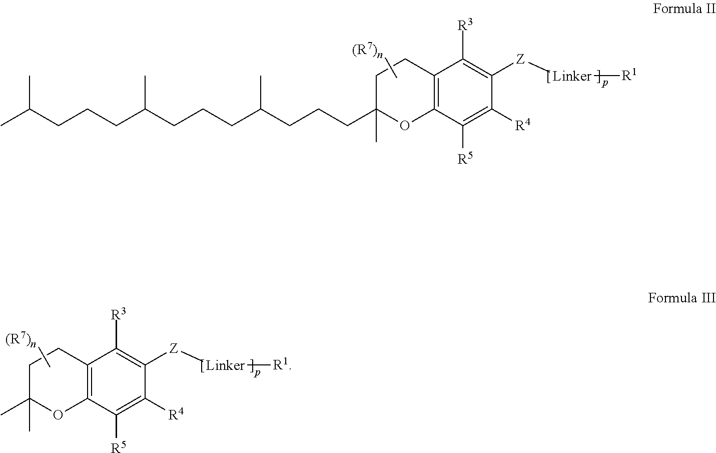

With respect to Formulas II and III, R¹, R³, R⁴, R⁵, R⁷, Z, Linker, n and p are as previously defined for Formula I.

In some aspects, the compound has a structure according to Formulas IIa, IIb, IIc, IId, IIIa, IIIb, IIIc, IIId, IVa, or IVb.

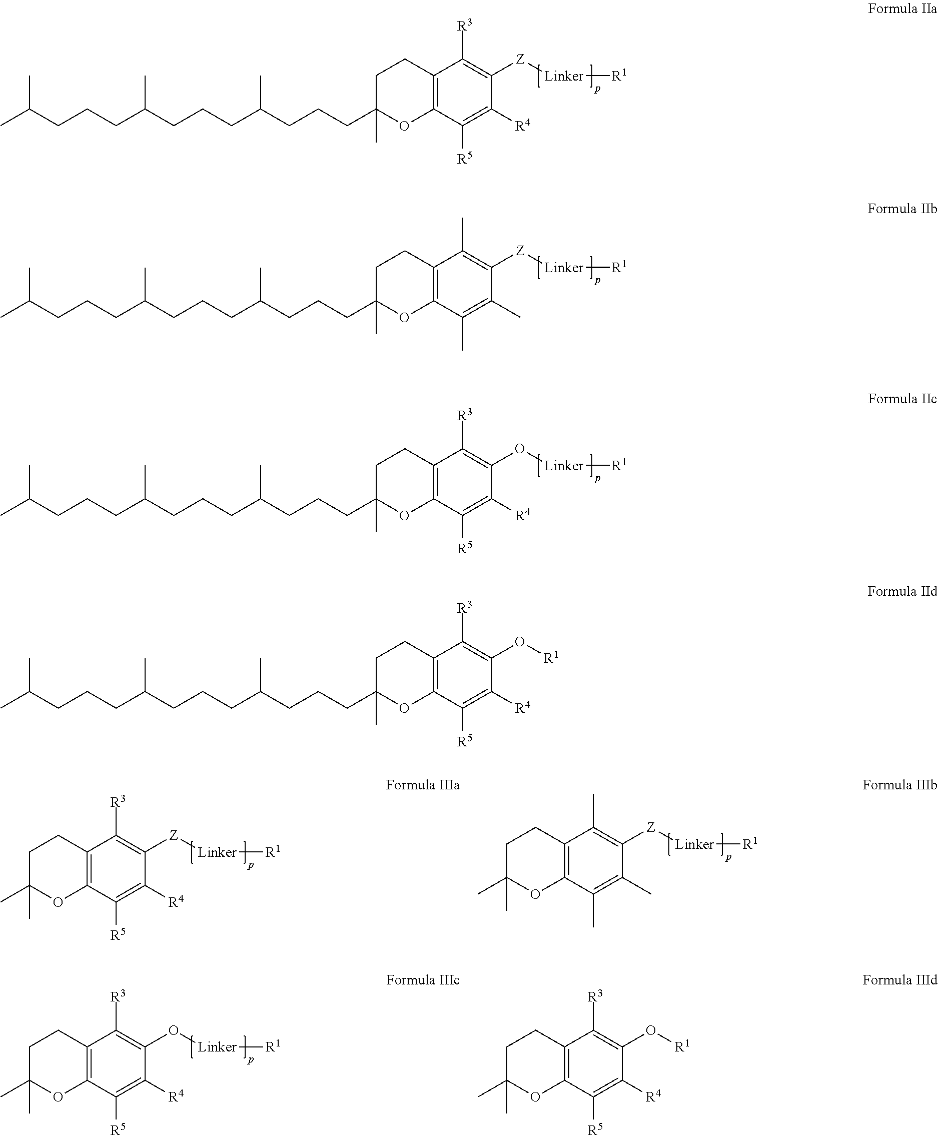

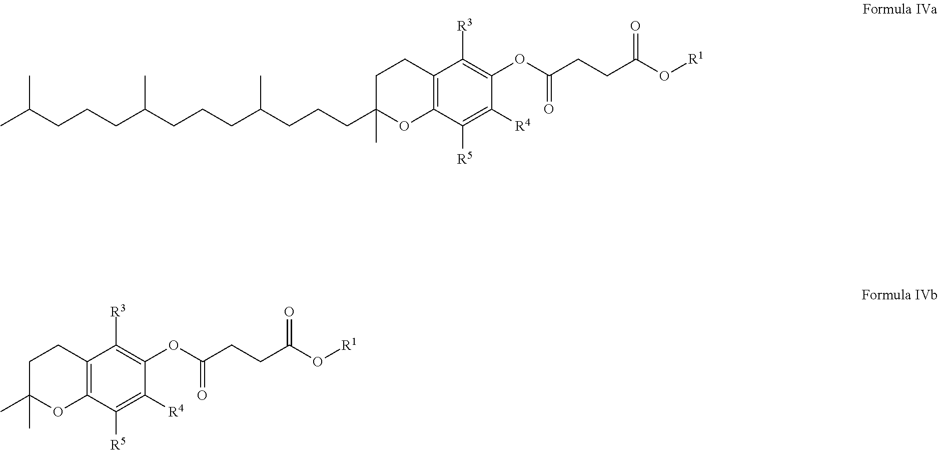

With respect to Formulas IIa, IIb, IIc, IId, IIIa, IIIb, IIIc, IIId, Iva, or IVb, R¹, R³, R⁴, R⁵, Z, Linker, and p are as previously defined for Formula I.

Certain disclosed exemplary compounds within the scope of Formula I include:

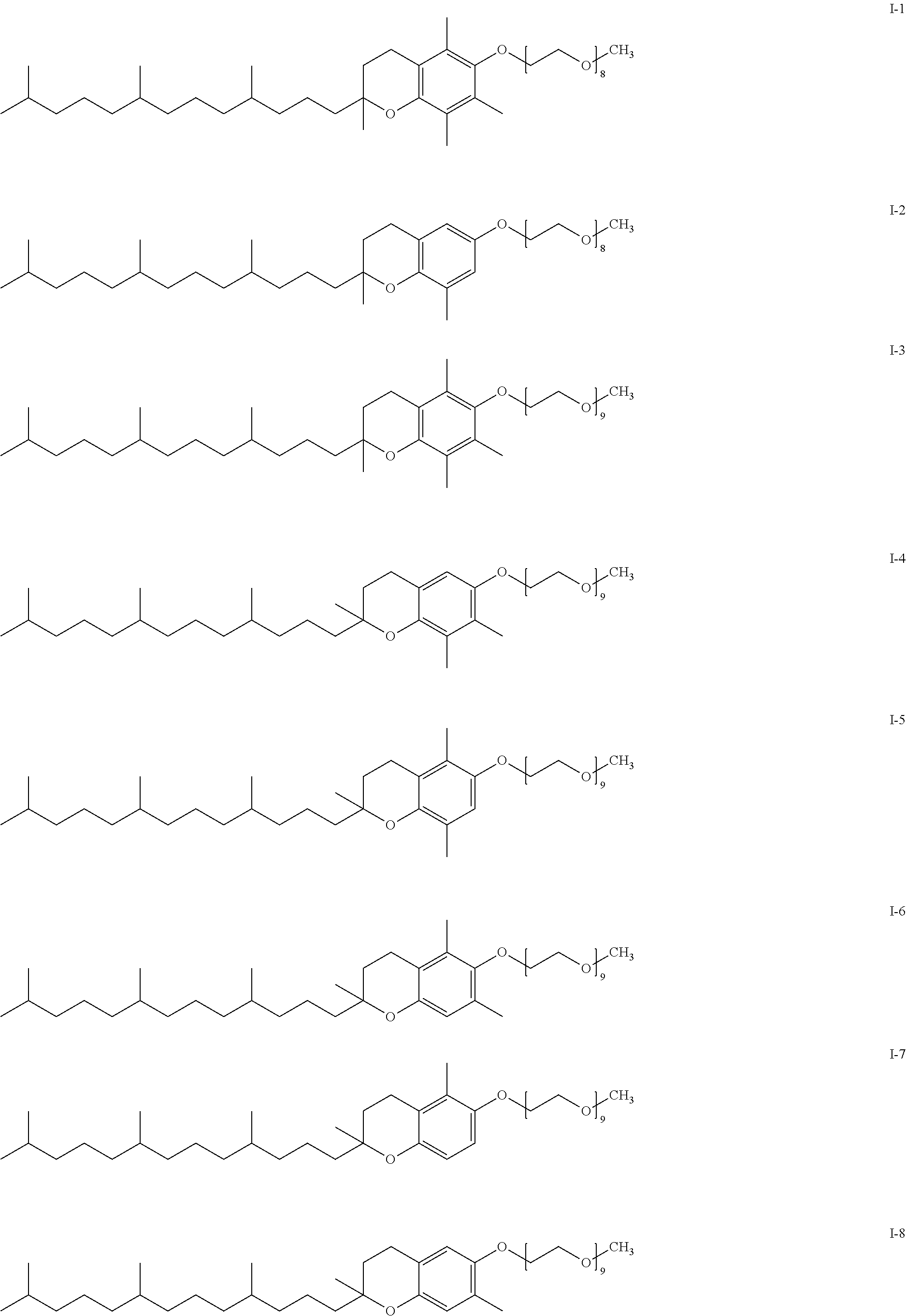

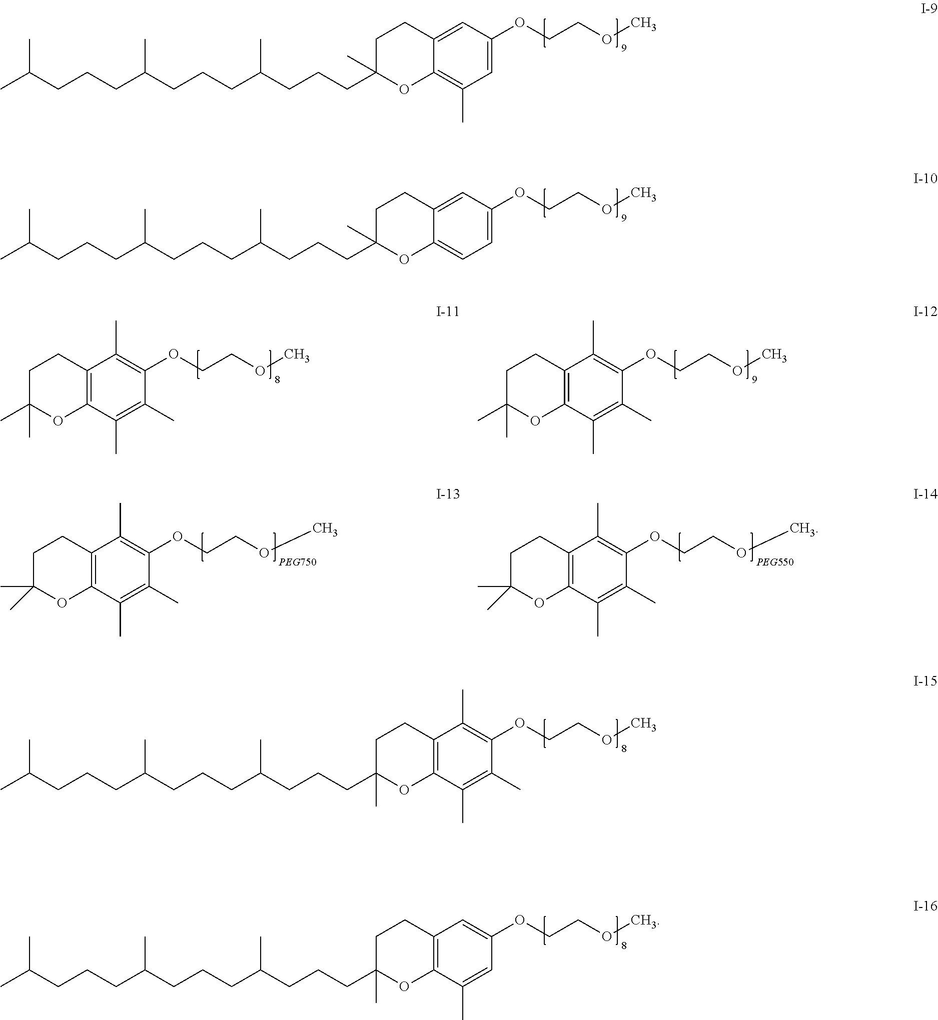

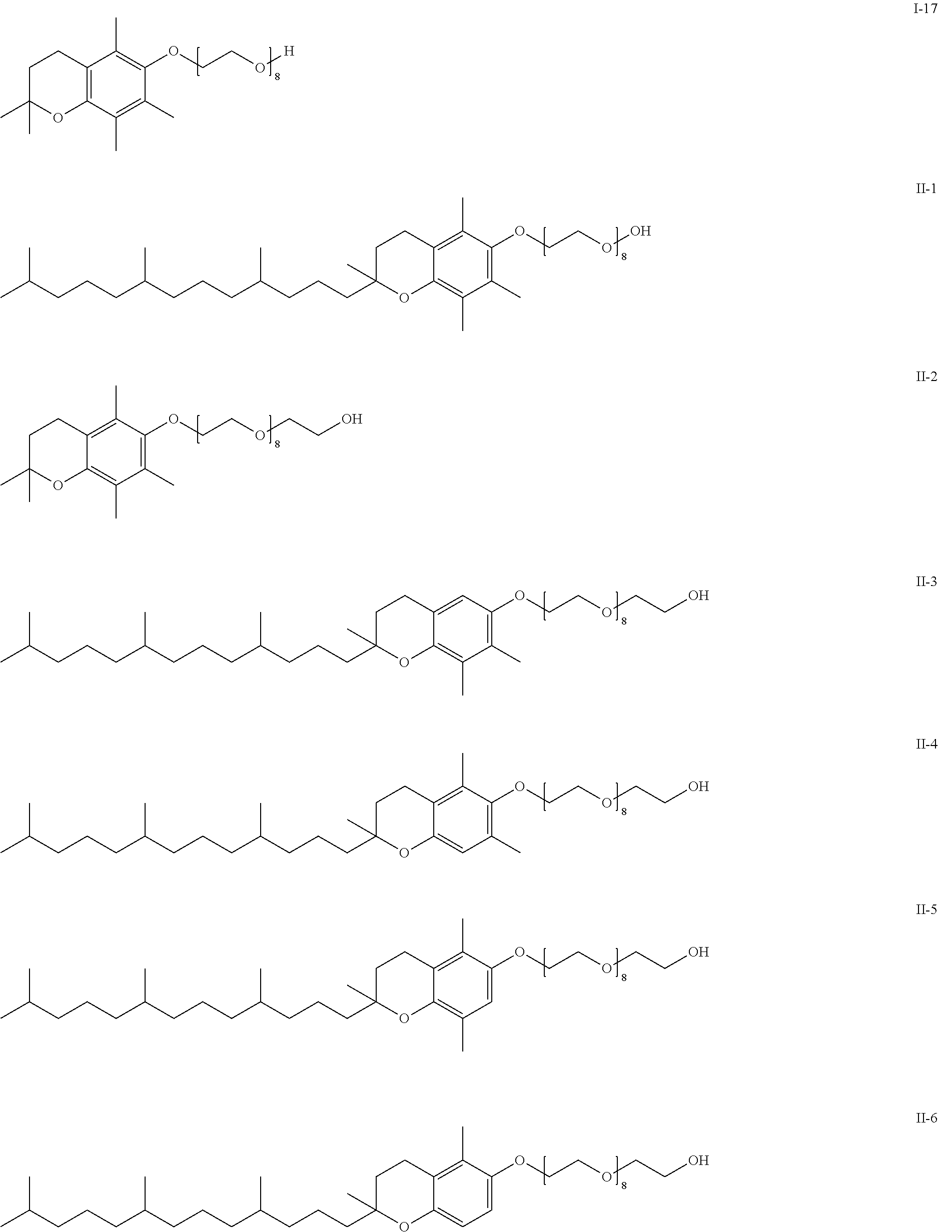

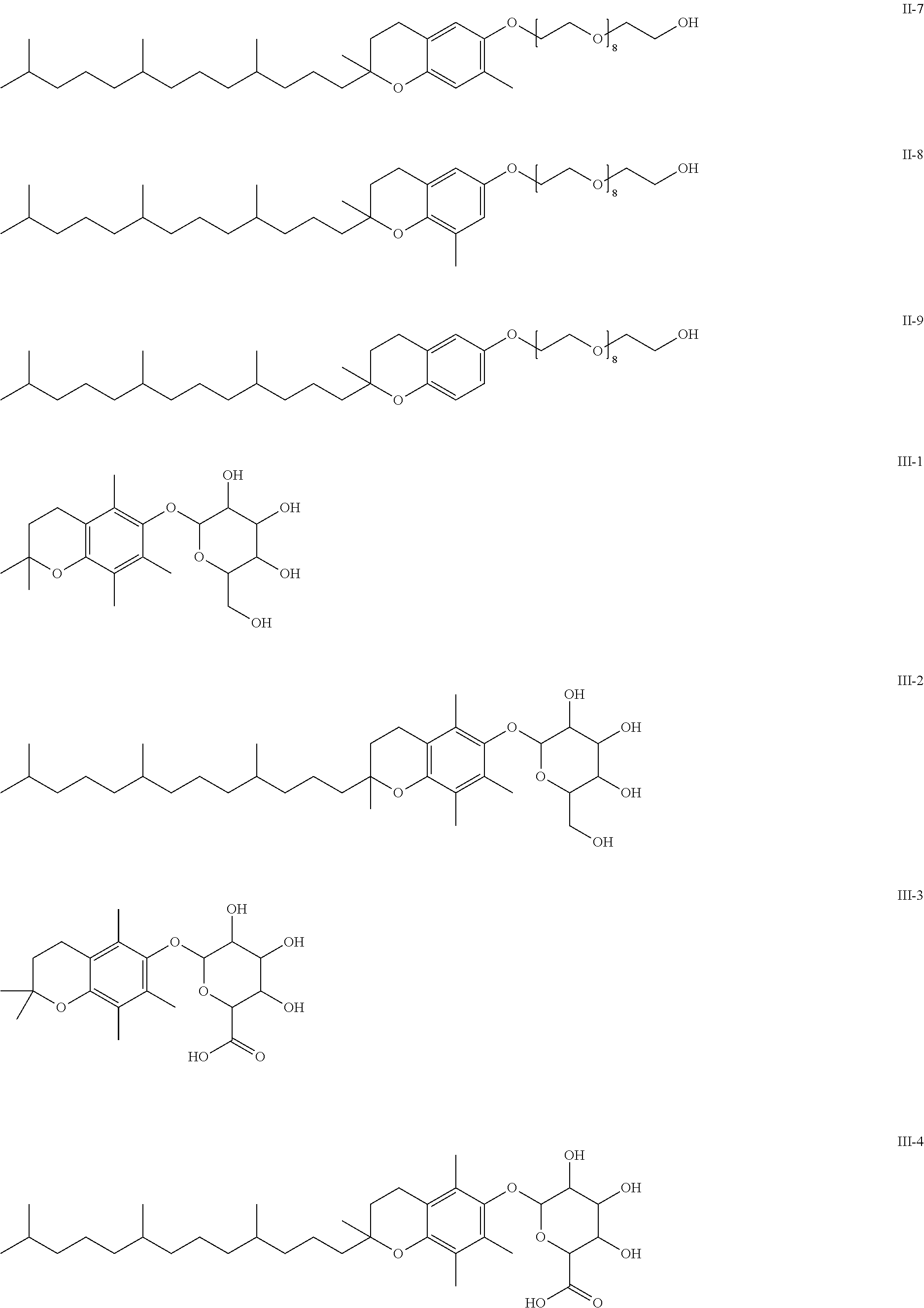

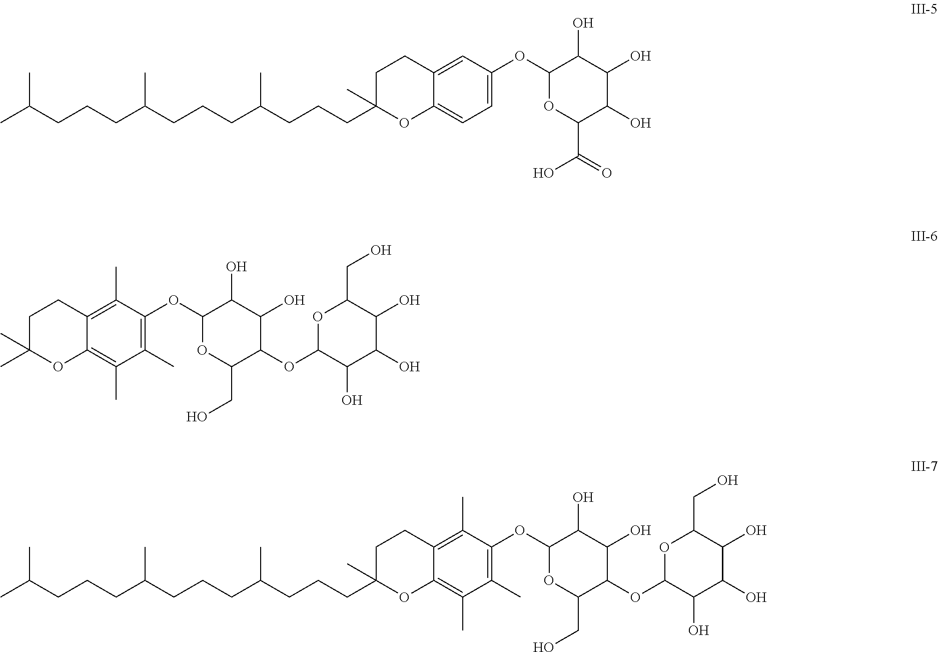 [SMILES: CC(C)CCCC(C)CCCC(C)CCCC1(C)CCc2cc(OC3OC(C(=O)O)C(O)C(O)C3O)ccc2O1 ; Cc1c(C)c2c(c(C)c1OC1OC(CO)C(OC3OC(CO)C(O)C(O)C3O)C(O)C1O)CCC(C)(C)O2 ; Cc1c(C)c2c(c(C)c1OC1OC(CO)C(OC3OC(CO)C(O)C(O)C3O)C(O)C1O)CCC(C)(CCCC(C)CCCC(C)CCCC(C)C)O2]

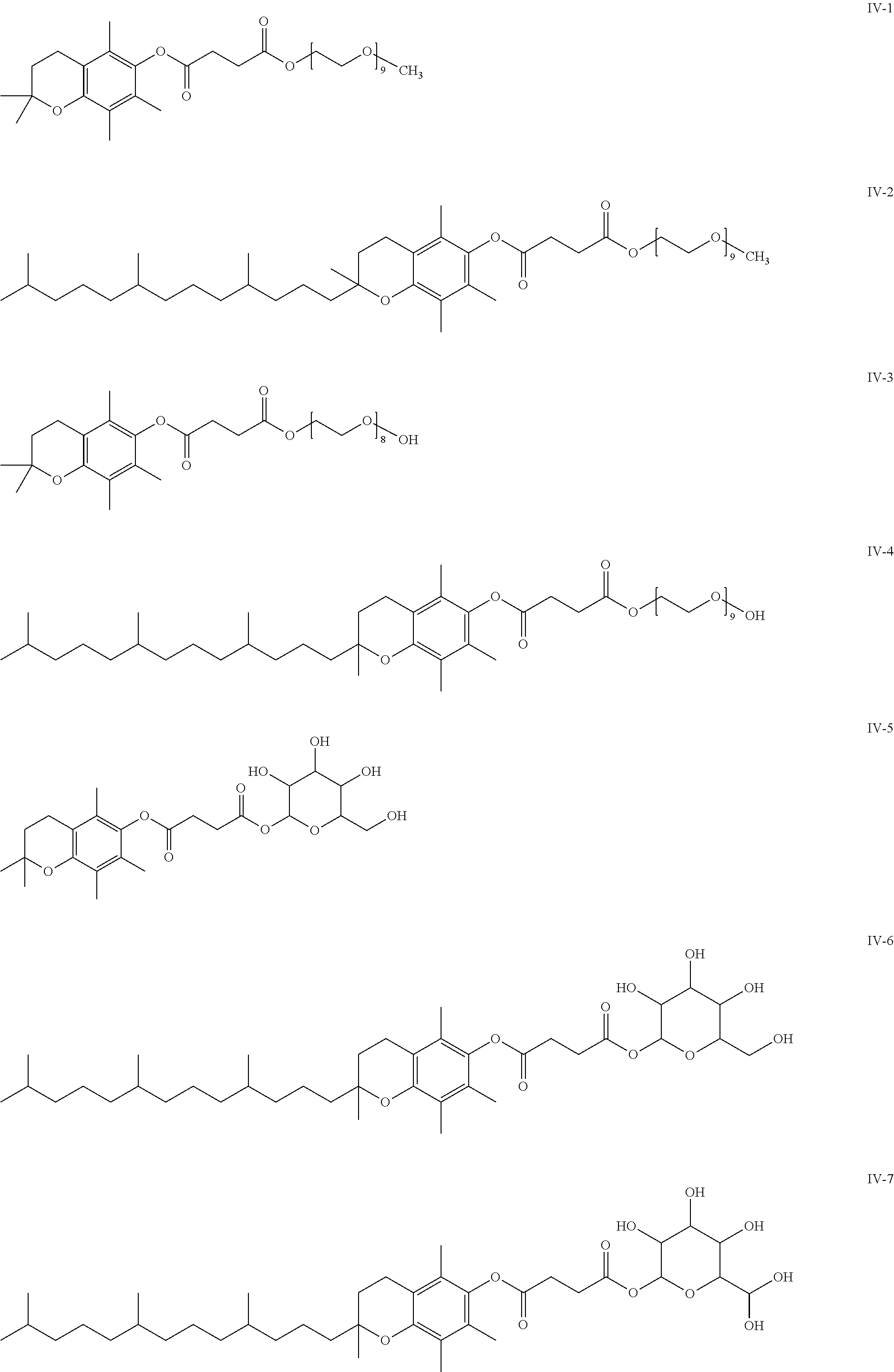

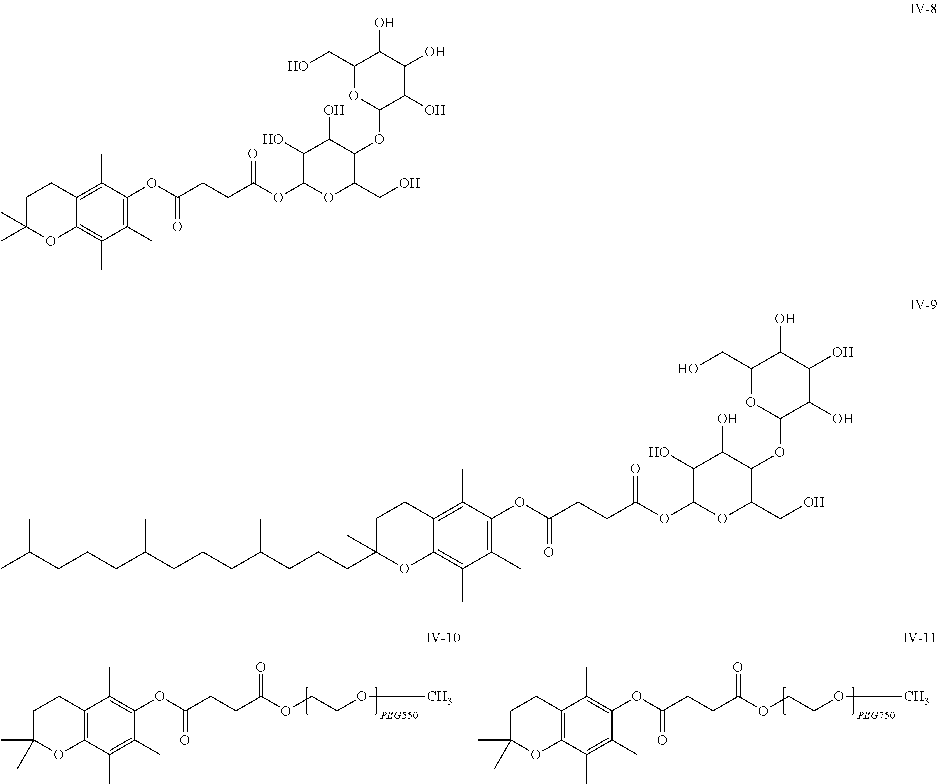

Also disclosed herein are aspects of a compound having a structure according to Formula V.

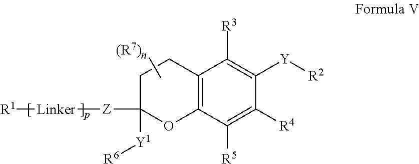

With respect to Formula V, R¹, R², R³, R⁴, R⁵, R⁶, R⁷, Y, Y¹, Z, Linker, n and p are as previously defined for Formula I.

In some aspects, Y is O.

In some aspects, R² is a straight alkyl chain (for example, up to 12 carbons in length), an aromatic or benzyl group (for example, phenyl or —CH₂phenyl), or a branched alkyl chains, for example

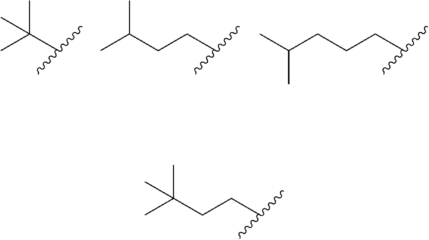

### IV. Synthesis

Disclosed compounds can be prepared as exemplified below, as illustrated for specific compounds in the examples, and as will be understood by a person of ordinary skill in the art of organic synthesis. An exemplary synthesis may include the following reaction step according to Scheme 1.

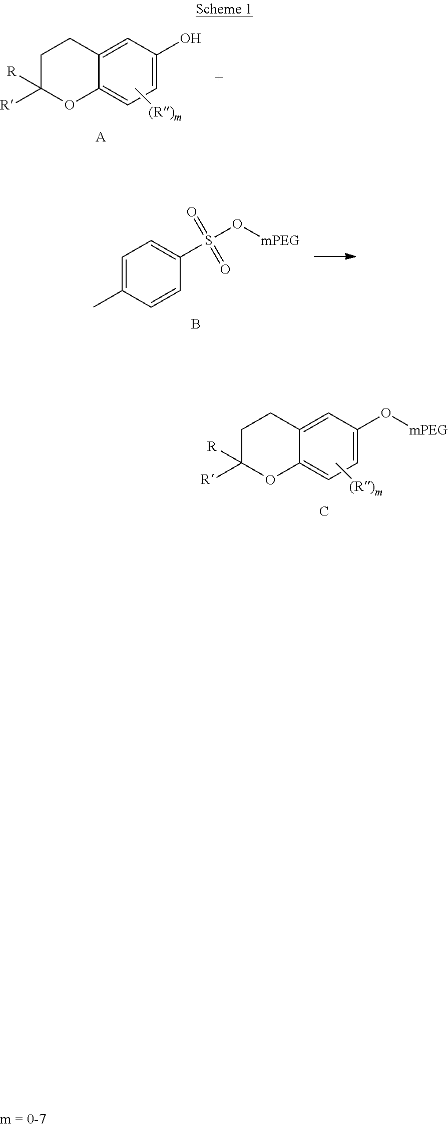

With respect to Scheme 1, compound A is treated with mPEG tosylate (Tos) compound B in the presence of a suitable base, such as sodium hydride, in a suitable solvent. Suitable solvents include, but are not limited to, aprotic solvents, such as tetrahydrofuran, acetonitrile, an alkyl ether (for example diethyl ether), toluene, hexanes, cyclohexane, or a combination thereof. The reaction is allowed to proceed at a suitable temperature, such as from 15 °C to 50 °C, or room temperature. Upon completion, the crude product is extracted from the reaction mixture and purified, such as by column chromatography.

An alternative exemplary synthesis may include the following first reaction step according to Scheme 2.

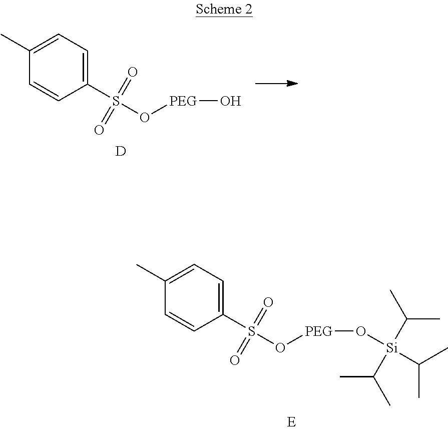

With respect to Scheme 2, tosylated PEG compound D is treated with triisopropylsilyl chloride (TIPS) in the presence of imidazole. Suitable solvents for this reaction include aprotic solvents, such as tetrahydrofuran, acetonitrile, an alkyl ether (for example diethyl ether), toluene, hexanes, cyclohexane, or a combination thereof. Typically the addition of the TIPS proceeds with cooling to a temperature between 25 °C and 0 °C, such as between 10 °C and 0 °C. After the addition, the reaction is allowed to warm to ambient temperature. Upon completion the product is isolated by a suitable technique, such as column chromatography.

A second step in the alternative synthesis proceed according to Scheme 3.

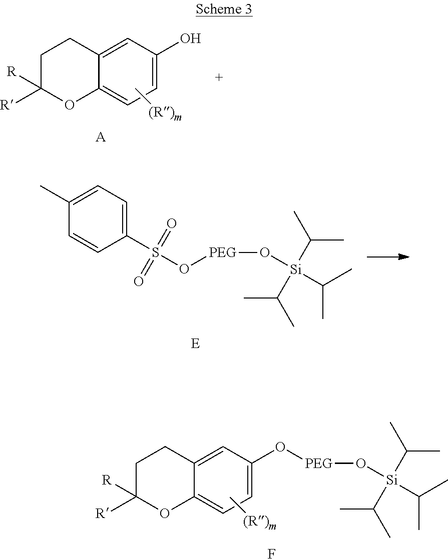

With respect to Scheme 3, compound A is treated with compound E in the presence of a suitable base, such as sodium hydride, in a suitable solvent. Suitable solvents include, but are not limited to, aprotic solvents, such as tetrahydrofuran, acetonitrile, an alkyl ether (for example diethyl ether), toluene, hexanes, cyclohexane, or a combination thereof. The reaction is allowed to proceed at a suitable temperature, such as from 15 °C to 50 °C, or room temperature. Upon completion, the crude product is extracted from the reaction mixture and purified, such as by column chromatography.

A third step in the alternative synthesis proceeds according to Scheme 4.

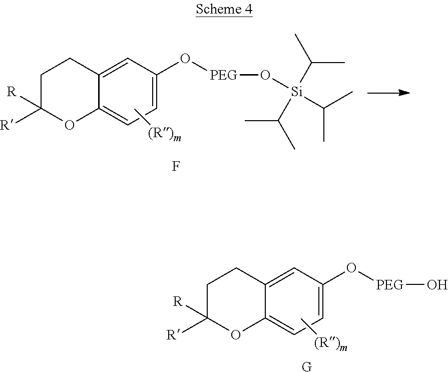

With respect to Scheme 4, compound F is dissolved in a suitable solvent, such as tetrahydrofuran (THF), and water is added. After cooling to below 10 °C, Tetra-n-butylammonium fluoride (TBAF) is added dropwise as a solution in THF. After addition, the reaction is allowed to warm to room temperature. Upon completion, the product is isolated and purified, such as by column chromatography.

A second alternative synthesis to make certain exemplary compounds proceeds according to Scheme 5.

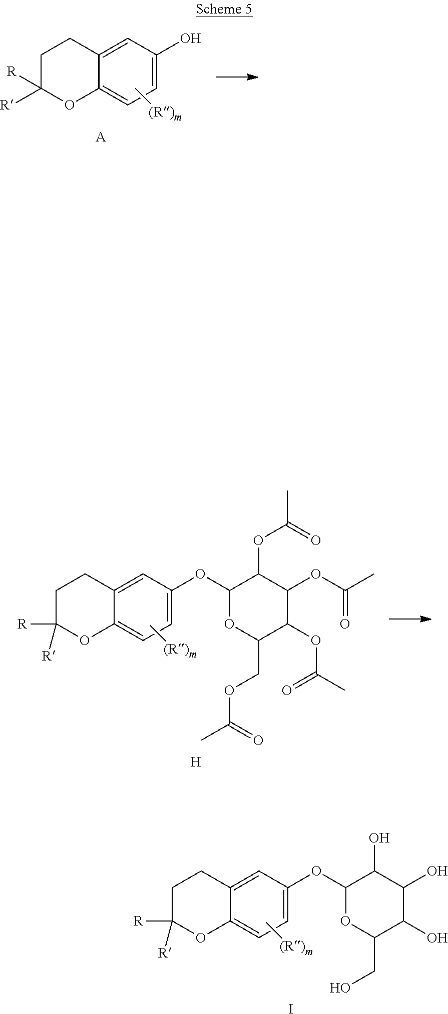

With respect to Scheme 5, α-acetobromogalactose is dissolved in a suitable anhydrous solvent and oven dried 3 angstrom molecular sieves are added. Suitable solvents include, but are not limited to, aprotic solvents, such as toluene, hexanes, cyclohexane, THF, or a haloalkane, for example, dichloromethane, dichloroethane, chloroform, and the like. Compound A is added followed by portionwise addition of a suitable base, such as a carbonate base, for example, silver carbonate. The reaction is allowed to proceed at a suitable temperature, such as from 15 °C to 50 °C, or room temperature. Upon completion, the reaction mixture is filtered, and compound H is isolated by a suitable technique, such as column chromatography.

Compound H is then dissolved in a suitable solvent and cooled to between 0 °C and 10 °C. Suitable solvents include, but are not limited to, aprotic solvents, such as tetrahydrofuran, acetonitrile, an alkyl ether (for example diethyl ether), toluene, hexanes, cyclohexane, or a combination thereof. A solution of lithium hydroxide in water is added dropwise. After the addition, the reaction is allowed to warm to room temperature. Upon completion, the product is isolated and purified, such as by column chromatography.

Additional exemplary compounds may be prepared according to a third alternative synthesis that proceeds according to Scheme 6.

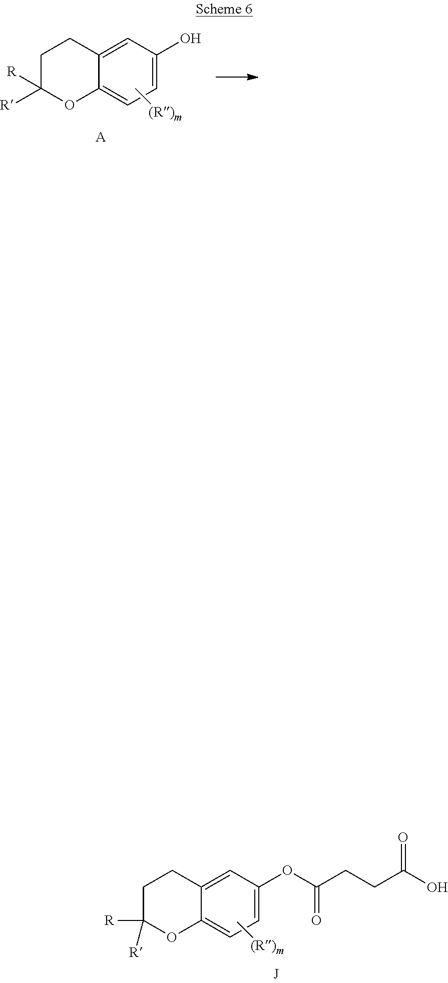

Compound A is treated with 4-dimethylaminopyridine (4-DMAP) and succinic anhydride in a suitable solvent. Suitable solvents include, but are not limited to, aprotic solvents, such as tetrahydrofuran, acetonitrile, an alkyl ether (for example diethyl ether), toluene, hexanes, cyclohexane, or a combination thereof. Upon completion, compound J is isolated and purified, such as by column chromatography.

A second reaction step in the third alternative synthesis proceeds according to Scheme 7.

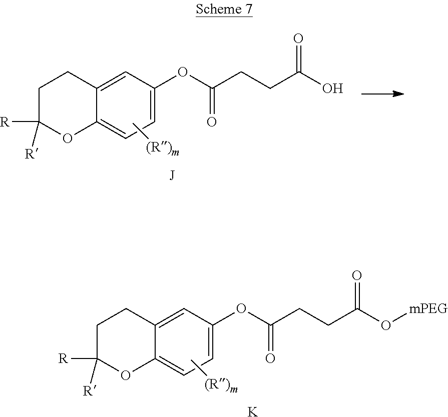

With respect to Scheme 7, compound J is treated with 4-DMAP and an m-PEG-OH compound in a suitable anhydrous solvent at room temperature. Suitable solvents include, but are not limited to, aprotic solvents, such as tetrahydrofuran, acetonitrile, an alkyl ether (for example diethyl ether), toluene, hexanes, cyclohexane, or a combination thereof. N,N-diisopropylcarbodiimide is the added dropwise. Upon completion, compound K is isolated and purified, such as by column chromatography.

With respect to solid-support (SS) bound surfactants, appropriately functionalized hydroxy-substituted tocopherol derivatives, carboxylic acid substituted or hydroxymethyl-substituted tocopherol derivatives are converted to PEG-linked constructs bearing terminal amine or hydroxyl groups. These pegylated intermediates are subsequently coupled to functionalized bead surfaces (for example, carboxyl- or epoxy-activated agarose or polystyrene beads or the like) to generate bead-immobilized non-ionic surfactant analogs.

Alternatively, polystyrene beads are modified with poly(ethylene glycol) linkers bearing terminal hydroxyl groups (PS-PEG-OH). The tocopherol derivatives are subsequently coupled to the PEG terminus via ester or carbonate linkages to yield PS-PEG-tocopherol derivatives as solid-support bound surfactants. Polystyrene beads functionalized with carboxylic acid groups

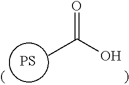

or hydroxy methyl groups

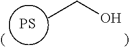

can be used to react with the PEG-alcohols to obtain polystyrene beads with PEG linkers bearing terminal carboxylic acid groups or terminal hydroxy groups.

### V. Applications

The disclosed compounds are useful as surfactants and can be used in many applications where a non-ionic surfactant is suggested or preferred. Applications include, but are not limited to, lysing cells, permeabilizing membranes, inactivating viruses, separating hydrophilic proteins from membranes, reducing surface tension, or decellularizing tissue. Additionally, the disclosed compound may be useful as an excipient or adjuvant in a vaccine, a cleaning agent, a component in buffers particularly biological buffers, a wetting agent, an emulsifier, a surfactant, or as a surface treatment for metals.

Compounds of this disclosure may be used in a broad range of products and applications across many industries, including but not limited to paint, textiles, metalworking, life sciences, pharmaceutical, industrial, environmental, construction, personal care, and specialty applications. Compounds of the present disclosure may also be useful in the manufacture of a wide range of products, serving as key components in creating surfactant mixtures, coatings, and emulsifier systems that require stable, effective surface activity.

Compounds of this disclosure may be useful as process and finishing aids in the manufacture of industrial raw materials and textiles across chemical, polymer, metallurgical, and biological industries, including as emulsifiers, oil-in-water emulsifiers, water-in-oil emulsifiers, wetting agents, phase transfer agents solubilization agents, viscosity modifiers, thickeners, gelling agents, foaming agents, foam stabilizers, antifoam agents, dispersion agents, micelle-forming agents, foam fractionation agents, cleaning agents, plasticizer systems, anti-static agents, anti-fog agents, anti-block additives, mold-flow modifiers, and resin stabilizers. In other aspects, compounds of this disclosure may be used in the formulations of hydrogels, aerogels, superabsorbent polymers, and polymers. In other aspects, compounds of this disclosure may also be used in biotechnology and enzyme production, such as in fermentation media for biotechnology and enzyme production; in polymer and plastic manufacturing; in metal and mineral processing, for example as collectors, frothers, or modifiers in mineral flotation systems, in degreasing and pickling baths during metal fabrication processes, in slag foaming, flux aids, in pickling and passivation processes, in metal extraction processes, and in metalworking fluid processes; in production of construction materials, such as cements, paints, coatings, and ceramic; in bio-based raw material manufacturing, for example in biorefineries and oleochemical plants; in manufacture, production and finishing of textiles, including leather; in pigment and filler dispersion manufacturing, for example to wet, stabilize, and uniformly distribute solid particles within liquid systems; in paper and pulp manufacturing including for purposes such as deinking, fiber wetting, and cleaning aids; and in water and utility systems used in manufacturing process, including as wetting agents, antifoams, defoamers, and dispersants.

In some aspects, compounds of this disclosure may also be used in the manufacture of polymers and resins; for example, they may be used in emulsion polymerization processes to make a wide range of polymers and resins used in a variety of applications, such as adhesives, paints, paper coating and textile coatings, and monodispersed particles and beads. In other aspects, compounds of this disclosure may be used in controlled radical polymerization processes, including atom transfer radical polymerization, reversible addition-fragmentation chain transfer, and nitroxide-mediated polymerizations, to stabilize dispersed phases, control particle size, solubilize monomers and catalysts, prevent coagulation, and enable high conversion polymerizations in aqueous emulsion, dispersion, and mini-emulsion systems. In other aspects, compounds of this disclosure may be used extensively in electronics and semiconductor manufacturing processes to control wetting behavior, enhance particle removal, stabilize abrasive or nanoparticle dispersions, improve uniformity of photoresist and coating layers, facilitate etching and post-etch cleaning, assist in metal deposition and plating processes, and support defect reduction across wafer fabrication steps. For example, compound of this disclosure may be used in semiconductor cleaning solutions, photoresist stripping formulations, photo developer formulations, etching formulations, chemical mechanical planarization (CMP) slurries, electrode slurries, post-CMP cleaners, wafer cleaners and mask cleaners. In other aspects, compounds of this disclosure can be used in printed circuit board manufacturing baths and cleaners. In other aspects, compounds of this disclosure may be also used in display manufacturing, including coating processes for organic light emitting diodes and light emitting diodes.

In some aspects, compounds of this disclosure may also be used in the manufacturing of batteries, supercapacitors, fuel cells and solar cells. In some aspects, compounds of this disclosure may be used in energy storage and solar technologies to control interfacial behavior, improve material dispersion, and enhance manufacturing consistency across a range of device architectures.

In some aspects, for example in batteries, supercapacitors, and fuel cells, compounds of this disclosure may function as electrode slurry and binder dispersants that enable uniform distribution of active materials, conductive additives, and polymers, leading to improved electrode homogeneity and performance. In some aspects, compounds of this disclosure may be incorporated into separator coatings and electrolyte formulations to modify wettability, stabilize interfaces, and reduce defects. In some aspects, for example in solar cell manufacturing—including perovskite and thin-film photovoltaics—compounds of this disclosure may be used to assist in forming smooth, defect-free coatings, improving film morphology, and enabling reliable deposition processes. Through these roles and others, compounds of this disclosure may be used support enhanced efficiency, stability, and manufacturability across modern electrochemical and photovoltaic energy devices.

In other aspects, compounds of this disclosure may be widely used in rubber, elastomer, latex, and tire manufacturing because they provide essential emulsification, wetting, and stabilization functions across multiple process stages. For example, in emulsion polymerized rubbers and acrylic latexes, compounds of this disclosure may be used to help disperse hydrophobic monomers, control particle size, and maintain latex colloidal stability under varying pH, temperature, and shear conditions. In solid rubber and tire compound mixing, compounds of this disclosure may act as processing aids that improve filler dispersion, enhance silica compatibility, reduce mixing energy, and support uniform incorporation of curatives and additives.

In latex dipping and rubber article fabrication, compounds of this disclosure may enable consistent wetting, film formation, foam control, and dispersion of pigments and compounding ingredients.

They are also used in post-vulcanization cleaning, mold release systems, and surface treatments to improve adhesion, appearance, and functional performance of finished rubber goods.

In some aspects, compounds of this disclosure may be widely employed in various advanced materials and chemical processing operations due, in part, to their ability to control interfacial phenomena, stabilize dispersed phases, and influence nucleation and growth behavior.

In crystal habit modification, in some aspects, compounds of this disclosure may be used to adsorb selectively onto specific crystal faces, thereby altering growth rates and enabling the production of crystals with tailored morphology, size, or purity. In nanoparticle synthesis, compounds of this disclosure may be used to act as steric stabilizers and structure-directing agents, preventing agglomeration of nucleating particles and supporting controlled growth of metal, metal oxide, polymeric, or inorganic nanoparticles. In solvent extraction processes, compounds of this disclosure may facilitate partitioning of hydrophobic solutes by enhancing solubilization and modifying phase boundaries, may be used in cloud point extraction to enable selective concentration or removal of target analytes. In membrane separation systems, compounds of this disclosure may be used as antifouling agents and wetting modifiers, improving membrane performance by reducing deposition of organic or biological materials, enhancing permeability, or stabilizing dispersed feed components. In other aspects, compounds of this disclosure may be used in eluents for chromatographic separations, in supercritical fluid extraction or in other advanced separation processes. In some aspects, for example in spray drying and spray granulation, compounds of this disclosure may be used to reduce surface tension and assist in droplet formation, particle agglomeration, and powder morphology control, improving flowability, solubility, and stability of resulting particulate products. In some aspects, compounds of this disclosure may be used in microencapsulation and controlled-release particle formation to stabilize emulsions or dispersions during capsule formation, promote uniform particle size, and enhance encapsulation efficiency of active ingredients. In some aspects, in heterogeneous catalysis, compounds of this disclosure may be used to stabilize catalyst nanoparticles, control pore structure in catalyst supports, and prevent sintering or deactivation by maintaining uniform dispersion under reaction conditions. Collectively, these functions highlight the versatility of compounds of this disclosure as interfacial engineering agents across diverse chemical, materials, and purification technologies.

In some aspects, for example in environmental and agricultural contexts, there is a particular need for non-ionic surfactants that are readily biodegradable and low-toxicity, for uses in eco-friendly cleaning agents, wastewater formulations, irrigation water treatments, and remediation products and other applications to reduce environmental persistence. In some aspects, compounds of this disclosure may be incorporated into formulations for pesticides, fungicides, herbicides, insecticides and plant growth regulators, including as a means to enhance the wetting and spreading of active ingredients on plant surfaces. They may also be used to facilitate improved adhesion and penetration of active compounds, reduce surface tension, and promote uniform coverage. In some aspects, compounds of this disclosure may be used as a spray mix adjuvant or as an evaporation control agent. In some aspects, compounds of this disclosure may also be used as adjuvants that stabilize emulsions, improve dispersion of micronized solids, and increase biological efficacy while reducing the need for volatile organic solvents. In some aspects, compounds of this disclosure may also be used as soil conditioning agents, as soil penetration enhancers, drift control agents, for example when spraying chemicals, seed treatment agents, and waste-to-energy feedstock conditioning agents. They also may be used in fertilizer coatings, seed coatings, seed treatment agents, and micronutrient delivery systems. In industrial water treatments, non-ionic surfactants may be used as anti-fouling agents, boiler treatment aids, and cooling tower dispersants. They may also be used in algal cultivation to improve dispersion, enhance nutrient and gas transfer, prevent cell aggregation, and maintain stable, uniform growth conditions. One of ordinary skill in this industry can readily appreciate the many uses for compounds of this disclosure, including in groundwater remediations like Surfactant-Enhanced Aquifer Remediation (SEAR), and soil washing; in wastewater and industrial effluent treatment; in bioremediation and microbial enhancement, to aid microbial degradation of hydrophobic pollutants; for environmental cleaning and decontamination applications, including spill response, surface decontamination, and industrial equipment and tank cleaning; and in analytical and environmental monitoring applications, including for example, in micellar electrokinetic chromatography or cloud point extraction, where they may be used for concentrating trace pollutants from water samples. In some aspects, compounds of this disclosure may be used in used in environmental treatment processes to enhance the separation and handling of contaminants in stormwater, runoff, landfill leachate, and sludge management applications. They may function as wetting, dispersing, and conditioning agents that improve solids-liquid separation, support sludge dewatering, and may be paired with foam-control technologies commonly used in wastewater treatment and aeration systems. They may also be used in dust suppression and erosion control applications, including as wetting agents to improve the penetration of water, as binding agents for dusty soils and unpaved roads, or to reduce airborne particulate emissions and soil erosion in places like mining or construction sites.

In other environmental applications. In some aspects, compounds of this disclosure may be used in foam fractionation, air stripping enhancements, and in ash wetting agents, like those used, for example, in coal utility processes. In some aspects, compounds of this disclosure may be used in air emission and odor control applications; for example, they may be incorporated into scrubbing solutions in gas treatment systems to capture volatile organic compounds or odorous compounds, by enhancing solubilization of hydrophobic gases or aerosols in the liquid scrubbing medium.

In some aspects, for example, in industrial applications, compounds of this disclosure may be used as pigment wetting agents and stabilizers in coatings and inks, contributing to improved flow, gloss, and film formation. In polymer chemistry, compounds of this disclosure may function as emulsifier mixtures in emulsion polymerization, where they may stabilize latex particles and influence particle size distribution, polymer morphology, and viscosity. In other aspects, compounds of this disclosure may be incorporated into automotive and architectural coatings, industrial lubricants and metalworking fluids, mold release agents, and corrosion inhibitor systems. They may be used to improve pigment dispersion, leveling, and surface smoothness; provide boundary lubrication, corrosion inhibition, and emulsification of oil-water mixtures; reduce surface tension and prevent adhesion between molds and formed materials; and stabilize protective films. In other aspects, compounds of this disclosure may be used in leather, hide or fur processing, including use during processes such as soaking, dehairing, degreasing, tanning, fatliquoring, dyeing and finishing. They may also be incorporated into leather treatment agents, waterproofing, softening or conditioning compositions.

In some aspects, compounds of this disclosure may be used in the construction sector, where a few examples include the integration of non-ionic surfactants into adhesives, sealants, and surface coatings; incorporated into materials such as flooring, tile, bathtubs, drywall, mirrors, insulations, semi-permanent fixtures, roofing materials, and asphalt and bitumen emulsions. Compounds of this disclosure may be incorporated into surface treatments and anti-corrosive finishes and act as dispersants, wetting agents and emulsion stabilizers for many applications, and may be used to improve adhesion to various substrates and the uniformity of polymer blends. In some aspects, compounds of this disclosure may be incorporated into concrete, mortar, or grout admixtures as superplasticizers, water reducers, or air-entraining agents.

In some aspects, compounds of this disclosure may be used in bio-decontamination and cleaning applications. In some aspects, they may be used in bio-spill response, for example in biocidal or cleaning formulations. In some aspects, compounds of this disclosure may be used in decontamination of medical, pharmaceutical, veterinary and lab equipment rooms; they may also be used in the decontamination of personal protective equipment and textiles such as gowns, masks, and reusable protective wear. In some aspects, they may be used in the formulation of liquid, paste, and powder cleaning products for industrial, institutional, and household use, including, for example, deodorizers, degreasers, aircraft cleaners, carpet and floor cleaners, dishwashing detergents and liquids, laundry detergents, pet shampoos, pet cleaners, fabric pre-treatment sprays, fabric softeners, fabric conditioners, disinfectant sprays and surface cleaners and wipes, sorbents and spill control kits, eye wash kits, drain and glass cleaners and products, air freshener products, polishes, leather degreasing and surface finishing agents. The low foaming tendency and chemical stability on compounds of this disclosure make them suitable for use for automated cleaning systems and high-performance degreasing applications. They may be used in solvents, chelating agents, and disinfectants, allowing for broad-spectrum cleaning and decontamination applications. Compounds of this disclosure may be used in auto body shampoos, polishes, and detailing products, and in formulations for cleaning and protecting interior surfaces and upholstery.

In some aspects, compounds of this disclosure may be used in specialty and occupational products, such as specialty coatings and surface treatments like aircraft paints, photographic developing chemicals, and medical or dental supplies. In other aspects, compounds of this disclosure may be incorporated into inks, glues, and craft adhesives to improve pigment dispersion, flow, and uniform drying. In 3D or additive manufacturing, it can be used in printing resins, printing inks, printing binders, printing powders, photopolymer resins, binder-jet inks, powder-bed wetting, post-processing cleaners. In writing instruments, compounds of this disclosure may be used to aid in the maintenance of ink fluidity and prevent clogging of delivery mechanisms.

In other aspects, compounds of this disclosure may be used in household maintenance products such as concrete patching compounds, primers, paint and finish removers, degreasers, paints, primers, surface sealants, septic system treatment products, antiscalants, and generally in products where there is a need to emulsify organic residues and promote biodegradation. In other aspects, compounds of this disclosure may be used in personal care products and cosmetics, including in products such as hair coloring, ostomy products, after-sun care products, wound care products, micellar water, shampoos, hair styling products, soaps, body washes and gels, bath and body oils, facial cleansers and moisturizers, perfumes, deodorants, anti-perspirants, sunburn treatment products, facial masks, body and face scrubs, toners, nail and cuticle care products, products for treating muscle or joint pain, makeup products, sterilization products, contact lens cleaners, lens wetting solutions, and lotions. In other aspects, compounds of this disclosure may be used in oral care products, such as in toothpaste, tooth powder, mouthwash, oral rinse, dental gel, denture cleaner, denture adhesives, interdental cleaners, and oral care foams; they may also be used in the coating for dental floss.

In other aspects, compounds of this disclosure may be used in autobody work products, such as cleaners, polishers, washes and shampoos. In other aspects, compounds of this disclosure may be used to act as a dispersant, friction modifier, antiwear additive, or emulsifier in many lubricants, such as engine oil, crankcase oil, gear oil, transmission fluid, hydraulic fluid, compressor oil, or grease.

In some aspects, compounds of this disclosure may be used in the food and beverage manufacturing and production industry for a number of applications, including improving texture, shelf life, and uniformity of food products. For example, they may be used as foaming agents, antifoaming agents, defoaming agents, food emulsification agents, and stabilizing agents. They may be used to create and stabilize emulsions between immiscible ingredients like oil and water; as solubilizers to enable homogenous blending; as dispersal agents, for example to disperse flavors, colors and vitamins in aqueous media; as antifoaming agents during food processes such as fermentation, bottling, or frying; as wetting and coating agents to facilitate even distribution, for example in food glazing, pan coatings, and dough conditioners; and in cleaning and sanitation products. Compounds of this disclosure may also be used in food packaging adhesives or food packaging coatings. In other aspects, compounds of this disclosure may be used in animal feed, pet food, aquaculture feed, or nutritional supplements; for example, they may be used as emulsifiers, stabilizers, or wetting agents in feed premixes, liquid feeds, or medicated feeds.

In some aspects, compounds of this disclosure may be used in pharmaceutical and biotechnological industries, where they may be used as solubilizers, emulsifiers, wetting agents, and stabilizers in pharmaceutical and biotechnological formulations. In some aspects, they may also be used as stabilizers and protectors, including to prevent crystallization, aggregation, or denaturation of proteins, vaccines, and biologics during formulation and storage. In some aspects, they may be used in pharmaceutical manufacturing, including as antifoaming and processing agents, or in pharmaceutical-grade cleaning formulations. Their mild nature allows for protein solubilization, membrane permeabilization, and stabilization of labile compounds. In some aspects, compounds of this disclosure may also be used as cryopreservation agents to stabilize cell membranes, reduce ice crystal-induced damage, and improve the uniform distribution and penetration of cryoprotective compounds during freezing and thawing. In some aspects, compounds of this disclosure may be used during manufacturing for various clean-in-place and sterilize-in-place operations; they may also be used in buffers during chromatographic purification, filtration, microfiltration or ultrafiltration steps. In some aspects, in veterinary and human pharmaceuticals, they may be used to improve bioavailability and stability of hydrophobic actives.

They may be included in drug delivery systems, biologics, and excipient formulations. They may also be used to prevent crystallization, aggregation, or denaturation of proteins, vaccines, and biologics during formulation and storage. In some aspects compounds of this invention may be used in various formulations, including parenteral formulations, such as injectables or intravenous emulsions, liposomes, or nanoemulsions; inhalation and nasal formulations; ophthalmic formulations, topical gels, creams or ointments; and in transdermal patches and suppositories.

In some aspects, compounds of this disclosure may be used in various aspects of medical device design, manufacture, and performance enhancement due, in part, to their ability to modify surface properties without imparting charge or ionic residues. Compounds of this disclosure may be used for cleaning and surface preparation of device substrates to remove contaminants and improve coating adhesion; as wettability enhancers and dispersing agents in hydrophilic or drug-eluting coatings; and as stabilizers in emulsions or polymer-drug formulations. Compounds of this disclosure may be used to improve biocompatibility by reducing protein adsorption and biofouling on device surfaces, while maintaining chemical stability through sterilization processes. They may be incorporated into coating compositions, processing baths, or formulation media to achieve controlled surface energy, uniform coating morphology, and consistent therapeutic performance across a range of medical devices such as catheters, stents, and implantable components. They may be used as surface-modifying primers and adhesion promoters for medical device assemblies. They may also be incorporated as components or adjuvants of biocides, antimicrobial coatings, surface disinfectants, or instrument cleaners.

In some aspects, compounds of this disclosure may be used in numerous applications in fuels, fuel production processes, and fuel additive technologies due to their ability to modify interfacial properties, enhance dispersion, and improve overall fuel system performance. For example, in gasoline, diesel, jet, marine, and biofuel formulations, compounds of this disclosure may be used as dispersants that help prevent or remove deposits on injectors, intake valves, and combustion surfaces, thereby maintaining engine cleanliness and efficiency. In other aspects, compounds of this disclosure may be incorporated into deposit-control additives and injector-cleaner packages to stabilize contaminants, solubilize gums, and reduce particulate agglomeration.

In other aspects, compounds of this disclosure may be used in in distillate fuels, for example to serve as cold-flow improvers that influence wax crystal morphology to enhance low-temperature operability, as well as demulsifiers that promote separation of water from fuel to protect storage and engine systems. Additionally, compounds of this disclosure may act as lubricity improvers, forming boundary films that reduce wear in low-sulfur and ultra-low-sulfur fuels. In other aspects, compounds of this disclosure may be used in fuel production processes, for example in desalting or treating processes. These diverse functionalities make compounds of this disclosure key components for optimizing stability, handling, and performance across modern fuel and fuel-additive applications.

In other aspects, compounds of this disclosure may be used widely in the energy, oil and gas, and mining industries, where they may be incorporated into numerous formulations including drilling muds and fluids, completion fluids, enhanced oil recovery (EOR) agents, demulsifiers, and cleaning compositions. In some aspects they may be used as dispersants and wax/paraffin inhibitors; as corrosion inhibitors and scale inhibitors for production and refining streams; as bitumen and heavy oil emulsifiers; or as oil sands processing aids. In some aspects, in enhanced oil recovery applications, compounds of this disclosure may be used to reduce interfacial tension between crude oil and injected aqueous phase, including to facilitate mobilization of residual oil otherwise trapped after primary and secondary recovery stages, to improve sweep efficiency during water flooding or chemical flooding operations, and increase total hydrocarbon recovery.

During crude oil production, compounds of this disclosure may be used in a number of ways, including as demulsifiers to destabilize and resolve unwanted emulsions which can form for a variety of reasons, including due to natural surface-active constituents such as asphaltenes, resins, and organic acids. They may be used to promote coalescence of dispersed water droplets and facilitate phase separation in separators and treaters. Compounds of this disclosure may also be incorporated into stimulation and cleaning treatments, including acidizing and hydraulic fracturing fluids. In these systems, compounds of this disclosure may reduce surface tension and alter rock wettability, enhancing fluid penetration into formation pores and removal of organic deposits such as paraffins or asphaltenes. In other aspects, in pipeline and equipment cleaning, compounds of this disclosure may be used as dispersants and detergents that emulsify hydrocarbon residues and sludge, thereby maintaining flow efficiency and mitigating corrosion.

In each of the foregoing applications, a person of ordinary skill in the art will readily understand how to choose from the compounds disclosed to select a compound with an HLB such that the surfactant is suitable for the use they intend. Moreover, a person of ordinary skill in the art will readily understand how to use routine testing for the selected compound of this disclosure to establish the parameters required for using the compounds of this disclosure in the various disclosed uses.

Nonionic surfactants, for example Triton™ X-100, Nonidet™ NP-40, and Tergitol™ NP-40, are employed widely across many industries. However, Triton™ X-100, Nonidet™ NP-40, and Tergitol™ NP-40 present environmental and toxicological concerns, including potential endocrine-disrupting properties, aquatic toxicity, and bioaccumulation associated with their structures. Regulatory restrictions—particularly within the European Union under REACH and environmental directives—have prompted the search for safer, high-performance alternatives.

Compounds of the present disclosure are such alternatives OECD 236 Fish Embryo Acute Toxicity (FET) test provides a standardized method for assessing the acute aquatic toxicity of chemical substances using early-life stages of fish. In this test, fertilized eggs-typically from Danio rerio (zebrafish)—are exposed to defined concentrations of a substance, and developmental endpoints such as coagulation, absence of somite formation, lack of heartbeat, and non-hatching are monitored over a 96-hour period. The resulting effect concentrations, including the LC50—the concentration of a substance causing lethal effects in 50% of embryos—offer quantitative measures of toxic potency that support regulatory hazard classification and environmental risk assessment. In some aspects, compounds of this disclosure may have a higher LC50 than Triton™ X-100, Nonidet™ NP-40, and Tergitol™ NP-40. In some aspects, compounds of this disclosure may have a LC50 at a concentration equal to or greater than 0.05, 0.1, 0.3, 0.5, 1, 2, 3, 5, 10, 15 or 20 mg/L.

Daphnia aquatic toxicity tests provide a standardized and sensitive method for evaluating the potential harmful effects of chemical substances on freshwater invertebrates, serving as an early indicator of ecological risk. In these assays, Daphnia magna or Daphnia pulex are exposed to defined concentrations of a test material under controlled conditions, and their immobilization or mortality is measured over a fixed period, typically 24 to 48 hours. The resulting effect concentrations, such as the EC₅₀—the concentration causing immobilization in 50% of the test organisms within 48 hours—enable quantitative comparison of toxic potency across substances and support regulatory classification of environmental hazards. By offering high reproducibility, well-characterized biological sensitivity, and rapid results, Daphnia tests play a crucial role in screening and differentiating product candidates, informing safe-by-design efforts, and ensuring compliance with international aquatic toxicity requirements. Triton™ X-100, Nonidet™ NP-40, and Tergitol™ NP-40 have a low EC50. By-products of Formula I result in significantly higher EC50 as compared to Triton™ X-100, Nonidet™ NP-40, and Tergitol™ NP-40 In some aspects, compounds of this disclosure have higher EC50—that is, they will not reach 50% immobilization until higher concentrations—as compared to Triton™ X-100, Nonidet™ NP-40, and Tergitol™ NP-40. In some aspects, compounds of this disclosure have an EC50 equal to or greater than 0.5, 1, 2, 3, 4, 5, 7.5, 10, 12, 15, or 20 mg/L, when tested under a Daphnia test.

The OECD 301B CO₂ Evolution Test provides a standardized and highly reliable method for determining the ready biodegradability of organic substances by quantifying the carbon dioxide evolved during microbial degradation under controlled aerobic conditions. By comparing the measured CO₂ production to the theoretical maximum expected from complete mineralization, the test delivers a clear, quantitative indication of a substance's environmental persistence. This method offers significant regulatory and practical benefits, including reproducible assessment across different chemical classes, early identification of environmentally benign product candidates, and support for compliance with global environmental safety requirements. The test's sensitivity and well-defined criteria enable developers to evaluate biodegradation performance with confidence, facilitating the design and selection of materials that minimize long-term ecological impact. A substance is determined to be “readily biodegradable” under OECD 301b if it has achieved a 60% biodegradability result, which is equivalent to 60% of ThOD (theoretical oxygen demand) or ThCO2 (theoretical carbon dioxide) production within a ten-day window during a 28-day test. Triton™ X-100, Nonidet™ NP-40, and Tergitol™ NP-40 have failed to achieve a 60% biodegradability result, leading to a desire to find effective non-ionic surfactants with a more favorable biodegradability profile. In some aspects, compounds of this disclosure may achieve a biodegradability result that is higher than Triton™ X-100, Nonidet™ NP-40, or Tergitol™ NP-40. In some aspects, compounds of this disclosure may achieve a 60% biodegradability result, as measured according to OECD 301b. In other aspects, compounds of this disclosure may have a biodegradability result that is equal to or greater than 20%, 25%, 30%, 40%, 50%, 60%, 65%, 70%, 75%, 80%, 85%, 90%, 95%, or 100% biodegradability result, as measured according to OECD 301b.

In one aspect, the disclosed compounds are useful in methods comprising cell lysis. In some aspects, the method comprises treating the cell with the disclosed compound(s) to facilitate cell lysis.

In one aspect, the disclosed compounds are useful in methods for virus inactivation, particularly inactivating a virus having a lipid envelope, or a virus that may develop a lipid envelope. In some aspects, a method for inactivating a virus comprises combining the disclosed compound with a liquid comprising the virus.

In other aspects, the disclosed compounds are useful for DNA or nucleic acid extraction such as from blood or pathogens applications; protein expression and/or purification; ELISA's; Western blotting; and RT_PCR applications.

Also disclosed herein are aspects of a kit comprising a compound disclosed herein.

### VI. Exemplary Aspects

The following numbered paragraphs illustrate exemplary aspects of the disclosed technology.

Paragraph 1. A compound having a structure according to Formula I

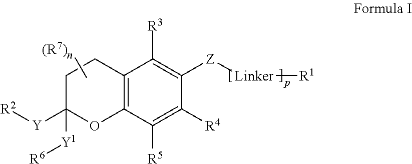

wherein:
   R¹ is a hydrophilic group;
R² is a lipophilic group;
each or R³, R⁴ and R⁵ independently is H, C₁₋₆alkyl, —OC₁₋₆alkyl, C₃₋₆cycloalkyl, or —OC₃₋₆cycloalkyl;
R⁶ is H or C₁₋₃₀alkyl;
R⁷ is C₁₋₆alkyl;
n is 0, 1, 2, 3, or 4;
Y is a bond, O, or N(R^{a});
Y¹ is a bond, O, or N(R^{a});
Z is O, N(R^{a}), C(═O), (C═O)N(R^{a}), C(═O)O, N(R^{a})C(═O), OC(═O), or a bond;
Linker is —(CH₂)ₓO—, —(CH₂)ₓC(═O)O—, —(CH₂)ₓC(═O)N—, —(CH₂)ₓOC(═O)—, or —(CH₂)ₓNC(═O)—;
x is 1, 2, 3, or 4;
p is 0 or 1; and
each R^{a} independently is H, C₁₋₄alkyl.

Paragraph 2. The compound of paragraph 1, wherein Y¹ is a bond.

Paragraph 3. The compound of paragraph 1 or paragraph 2, wherein R⁶ is H or methyl.

Paragraph 4. The compound of any one of paragraphs 1-3, wherein Y is a bond.

Paragraph 5. The compound of any one of paragraphs 1-4, wherein R² is C₁₋₂₅alkyl, C₂₋₂₅alkenyl, or C₂₋₂₅alkynyl.

Paragraph 6. The compound of any one of paragraphs 1-5, wherein R² is C₁₋₂₅alkyl.

Paragraph 7. The compound of any one of paragraphs 1-6, wherein R² is C₄₋₂₀alkyl.

Paragraph The compound of any one of paragraphs 1-7, wherein R² is

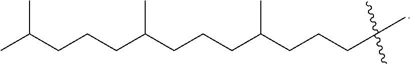 [SMILES: *CCCC(C)CCCC(C)CCCC(C)C]

Paragraph 9. The compound of any one of paragraphs 1-6, wherein R² is methyl.

Paragraph 10. The compound of paragraph 8 or paragraph 9, wherein R⁶ is methyl.

Paragraph 11. The compound of paragraph 10, wherein both of Y and Y¹ are bonds.

Paragraph 12. The compound of any one of paragraphs 1-11, wherein the compound has a structure according to Formula II or Formula III

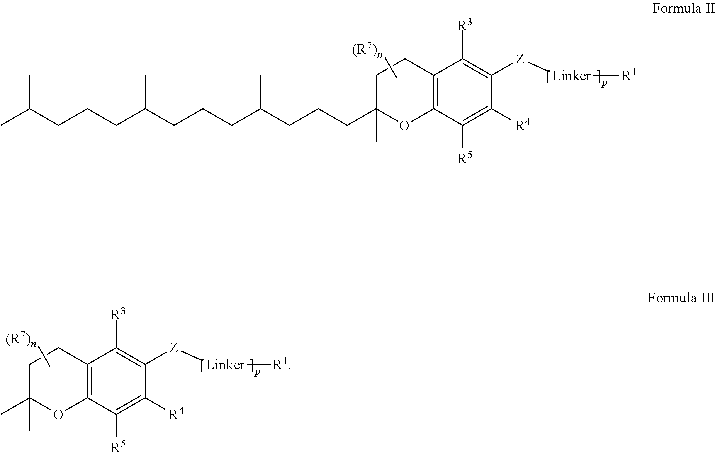

Paragraph 13. The compound of any one of paragraphs 1-12, wherein n is 0.

Paragraph 14. The compound of any one of paragraphs 1-12, wherein n is 1 or 2.

Paragraph 15. The compound of any one of paragraphs 1-14, wherein the compound has a structure according to Formulas IIa, IIb, IIc, IId, IIIa, IIIb, IIIc, IIId, Iva, or IVb.

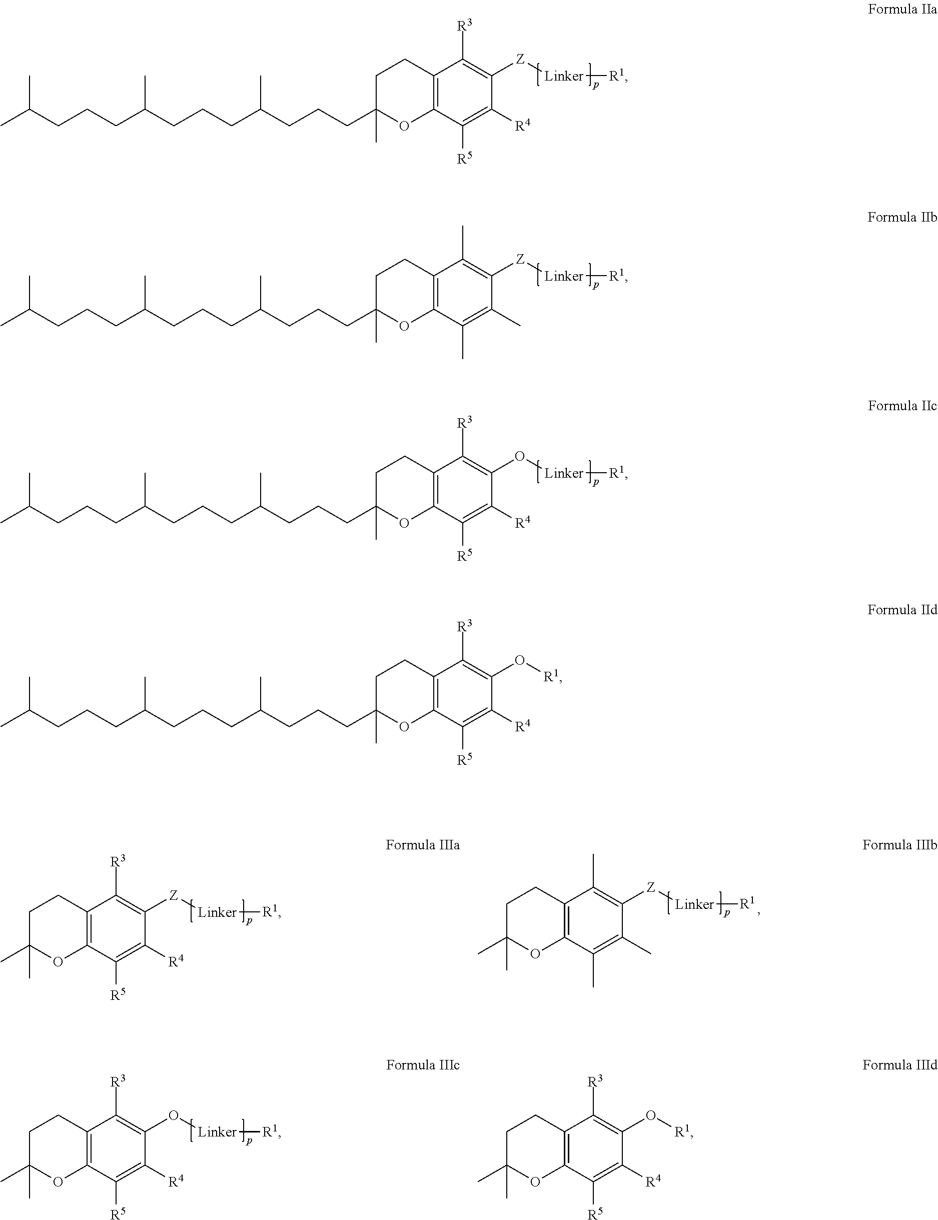

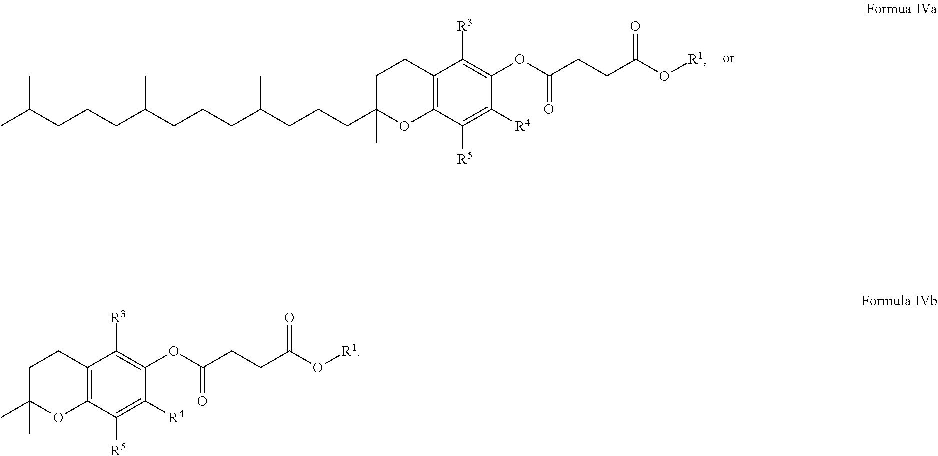

Paragraph 16. The compound of any one of paragraphs 1-15, wherein each of R³, R⁴ and R⁵ independently is H or C₁₋₆alkyl.

Paragraph 17. The compound of any one of paragraphs 1-16, wherein each of R³, R⁴ and R⁵ independently is H or methyl.

Paragraph 18. The compound of any one of paragraphs 1-17, wherein at least one of R³, R⁴ and R⁵ is not H.

Paragraph 19. The compound of paragraph 18, wherein two of R³, R⁴ and R⁵ are not H.

Paragraph 20. The compound of paragraph 18, wherein all three of R³, R⁴ and R⁵ are not H.

Paragraph 21. The compound of paragraph 20, wherein all of R³, R⁴ and R⁵ are methyl.

Paragraph 22. The compound of any one of paragraphs 1-21, wherein Z is O.

Paragraph 23. The compound of any one of paragraphs 1-22, wherein p is 0.

Paragraph 24. The compound of any one of paragraphs 1-22, wherein p is 1.

Paragraph 25. The compound of paragraph 24, wherein Z is OC(═O) and Linker is —(CH₂)ₓC(═O)O—.

Paragraph 26. The compound of paragraph 25, wherein x is 2 or 3.

Paragraph 27. The compound of any one of paragraphs 24-26, wherein the Z-Linker moiety is —OC(O)CH₂CH₂C(O)O—.

Paragraph 28. The compound according to any one of paragraphs 1-27, wherein R¹ is a polyalkylene oxide group.

Paragraph 29. The compound according to paragraph 28, wherein the polyalkylene oxide group is polyethylene glycol (PEG), polypropylene glycol (PPG), or a combination thereof, optionally terminated in an alkyl group.

Paragraph 30. The compound of paragraph 28 or paragraph 29, wherein:
R¹ is —([(CH₂)_{y}]-0)_{z}—R;
each y independently is 2 or 3;
z is from 4 to 25; and
R^{c} is H or C₁₋₄alkyl.

Paragraph 31. The compound of paragraph 30, wherein each y is 2.

Paragraph 32. The compound of paragraph 30 or paragraph 31, wherein R^{c} is H.

Paragraph 33. The compound of paragraph 30 or paragraph 31, wherein R^{c} is CH₃.

Paragraph 34. The compound of any one of paragraphs 28-33, wherein z is from 8 to 12.

Paragraph 35. The compound of paragraph 34, wherein z is 8 or 9.

Paragraph 36. The compound of any one of claims 28-35, wherein R¹ is a PEG or PPG group having a mass average molecular weight ranging from 500 to 800 daltons.

Paragraph 37. The compound of any one of paragraphs 1-27, wherein R¹ is a heterocycloaliphatic group.

Paragraph 38. The compound of paragraph 37, wherein the heterocycloaliphatic group is a 5- or 6-membered oxygen-containing heterocycloaliphatic group.

Paragraph 39. The compound of paragraph 37 or paragraph 38, wherein the heterocycloaliphatic group derived from a sugar moiety.

Paragraph 40. The compound of any one of paragraphs 37-39, wherein
R¹ is

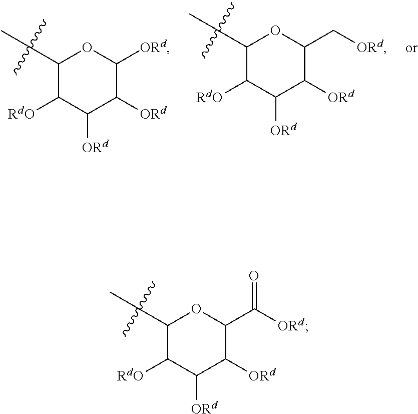

and
each R^{d} independently is H, a polyalkylene oxide group,

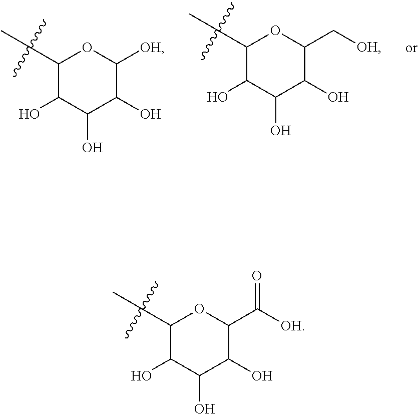 [SMILES: *C1OC(O)C(O)C(O)C1O ; *C1OC(CO)C(O)C(O)C1O ; *C1OC(C(=O)O)C(O)C(O)C1O]

Paragraph 41. The compound of paragraph 40, wherein the polyalkylene oxide group is polyethylene glycol (PEG), polypropylene glycol (PPG), or a combination thereof, optionally terminated in an alkyl group.

Paragraph 42. The compound of paragraph 40 or paragraph 41, wherein:
the polyalkylene oxide group is —([(CH₂)_{y}]—O)_{z}—R^{c};
each y independently is 2 or 3;
z is from 4 to 25; and
R^{c} is H or C₁₋₄alkyl.

Paragraph 43. The compound of paragraph 42, wherein z is 8 to 12.

Paragraph 44. The compound of paragraph 42, wherein z is 8 or 9.

Paragraph 45. The compound of paragraph 40, wherein each R^{d} is H.

Paragraph 46. The compound of paragraphs 40, wherein each R^{d} independently is

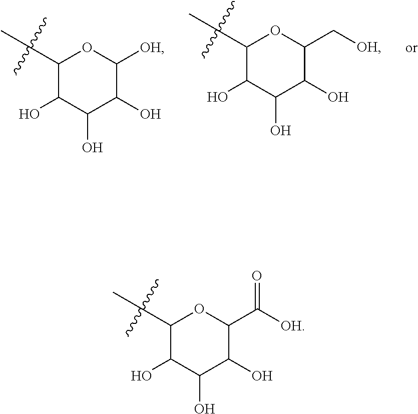 [SMILES: *C1OC(O)C(O)C(O)C1O ; *C1OC(CO)C(O)C(O)C1O ; *C1OC(C(=O)O)C(O)C(O)C1O]

Paragraph 47. The compound of any one of paragraphs 1-46, wherein R¹ and R² are selected to provide a hydrophilic-lipophilic balance (“HLB”) value ranging from 10 to 18.

Paragraph 48. The compound of paragraph 1, selected from

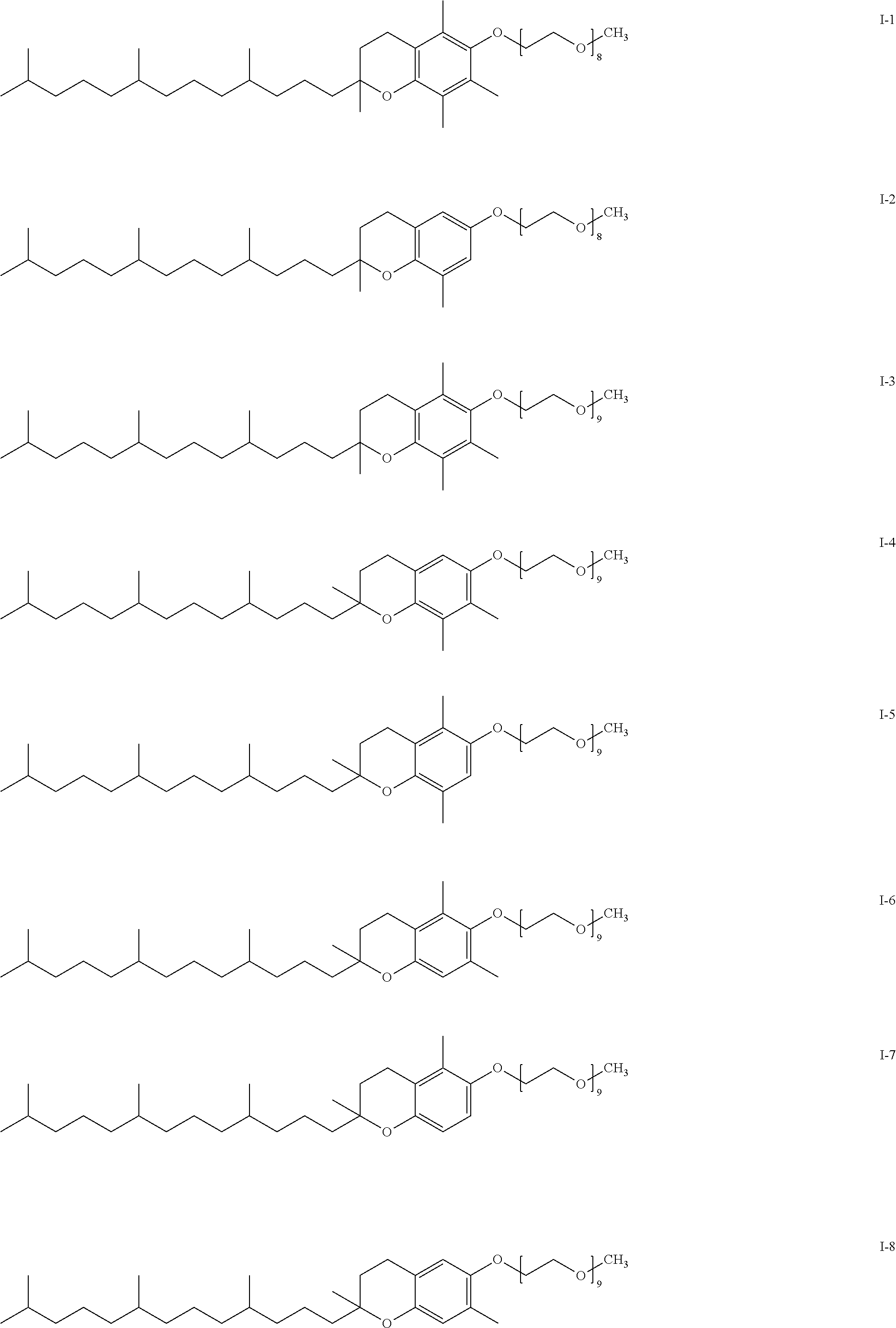

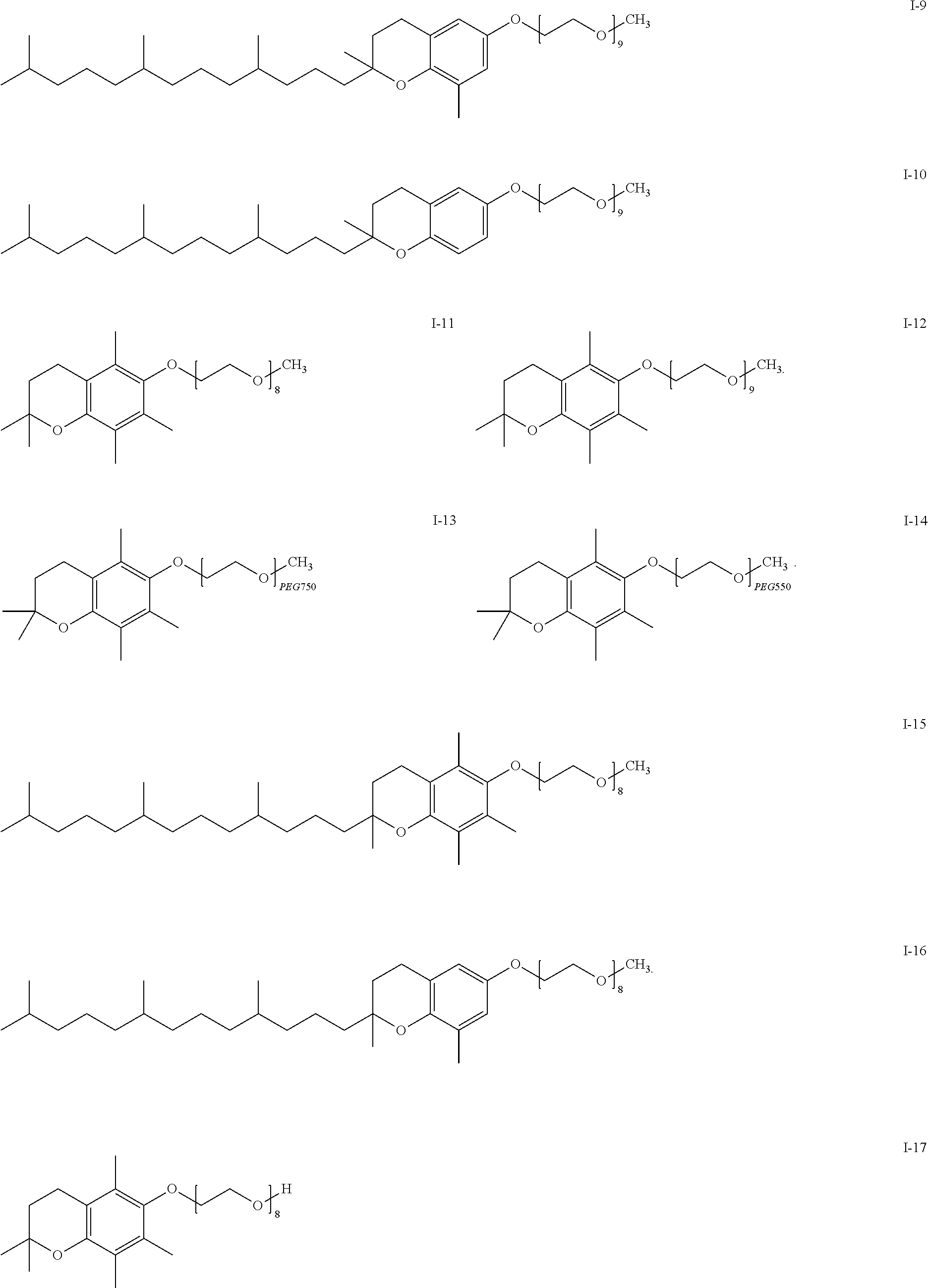

Paragraph 49. The compound of paragraph 1 selected from

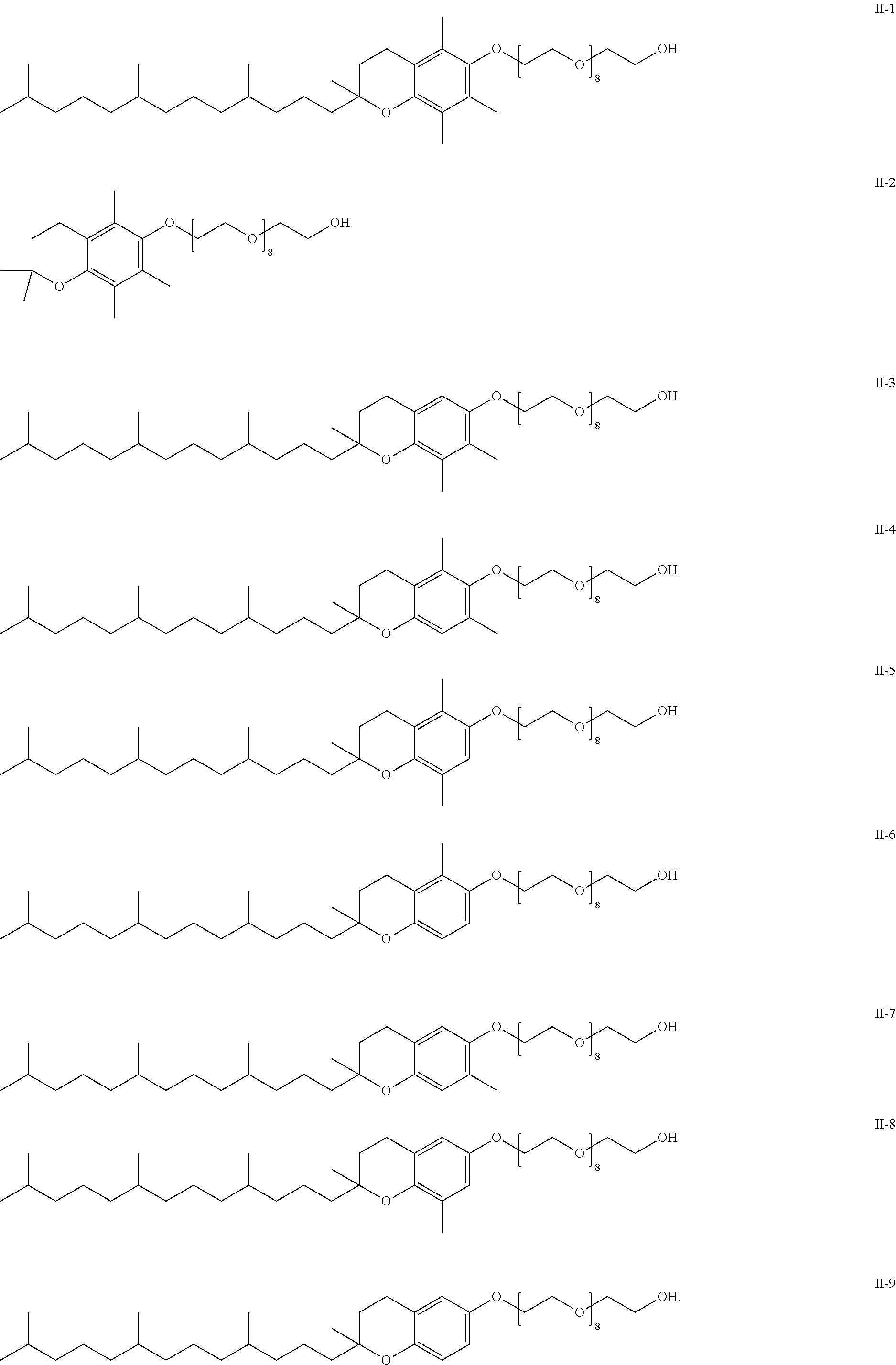

Paragraph 50. The compound of paragraph 1, selected from

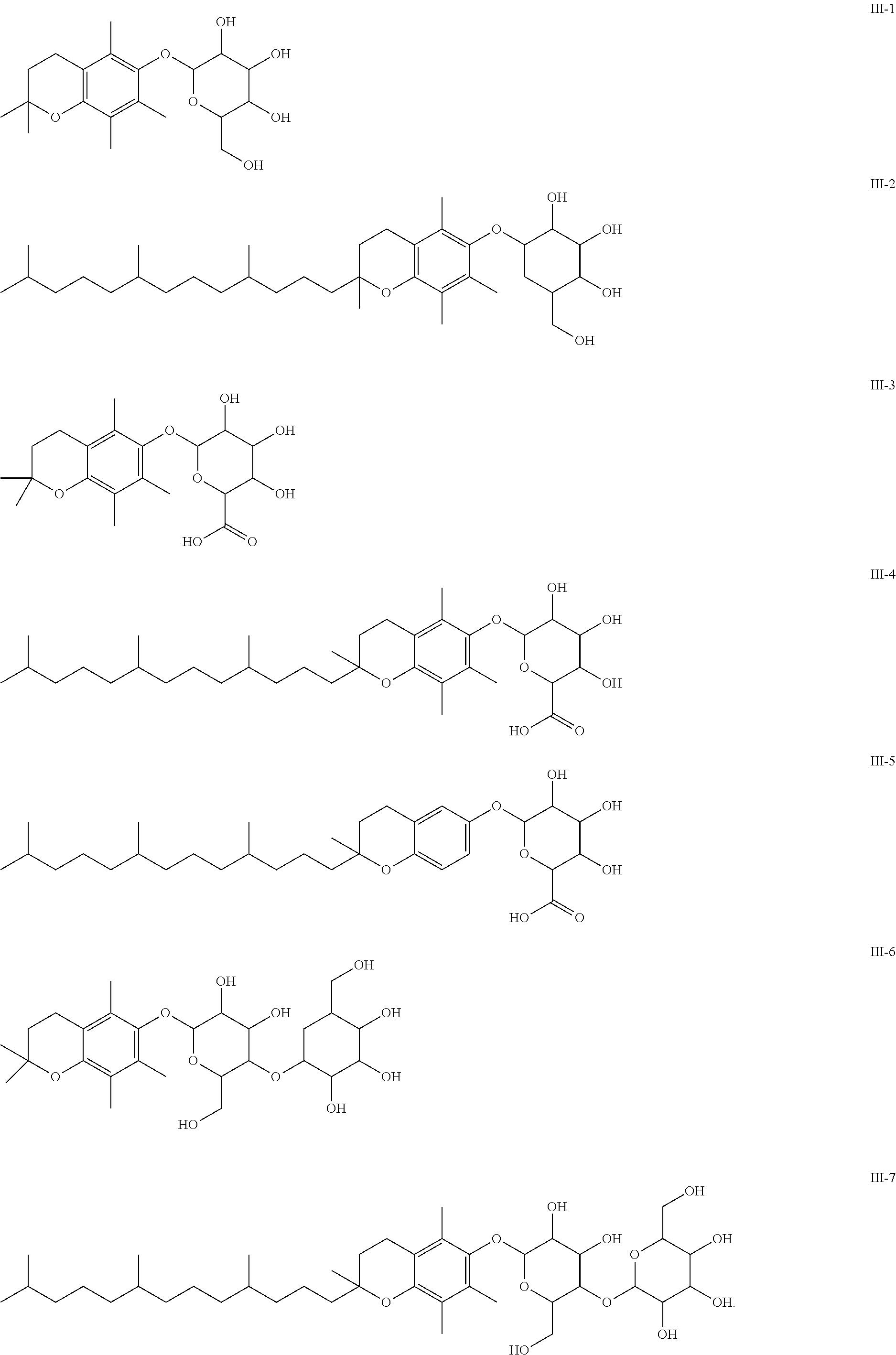

Paragraph 51. The compound of paragraph 1, selected from

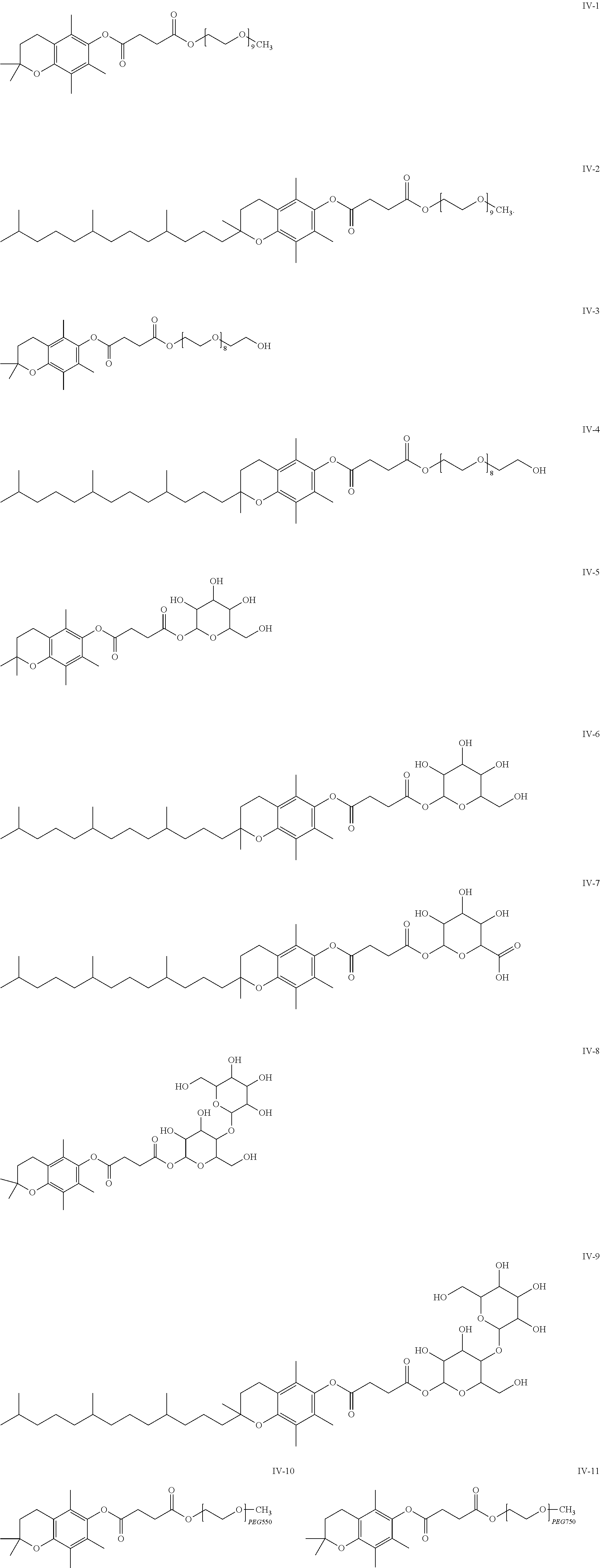

Paragraph 52. A compound having a structure according to Formula V

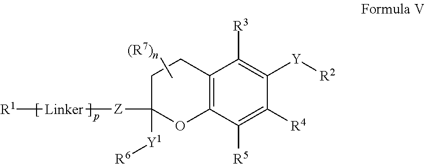

wherein:
   R¹ is a hydrophilic group;
R² is a lipophilic group;
each or R³, R⁴ and R⁵ independently is H, C₁₋₆alkyl, —OC₁₋₆alkyl, C₃₋₆cycloalkyl, or —OC₃₋₆cycloalkyl;
R⁶ is H or C₁₋₃₀alkyl;
R⁷ is C₁₋₆alkyl;
n is 0, 1, 2, 3, or 4;
Y is a bond, O, or N(R^{a}); Y¹ is a bond, O, or N(R^{a});
Z is O, N(R^{a}), C(═O), (C═O)N(R^{a}), C(═O)O, N(R^{a})C(═O), OC(═O), or a bond;
Linker is —(CH₂)ₓO—, —(CH₂)ₓC(═O)O—, —(CH₂)ₓC(═O)N—, —(CH₂)ₓOC(═O)—, or —(CH₂)ₓNC(═O)—;
x is 1, 2, 3, or 4;
p is 0 or 1; and
each R^{a} independently is H, C₁₋₄alkyl.

Paragraph 53. A method, comprising exposing a sample to a compound according to any one of paragraphs 1-52.

Paragraph 54. The method of paragraph 53, wherein the sample comprises a cell, optionally wherein the cell is solubilized and/or permeabilized.

Paragraph 55. A method of lysing a cell, comprising contacting the cell with an amount of a compound according to any one of paragraphs 1-52 sufficient to facilitate cell lysis.

Paragraph 56. A composition, comprising a compound according to any one of paragraphs 1-52.

Paragraph 57. A kit comprising a compound according to any one of paragraphs 1-52 and a container.

### VII. Examples

### Example 1

### General Procedure to Make m-PEG Analogs

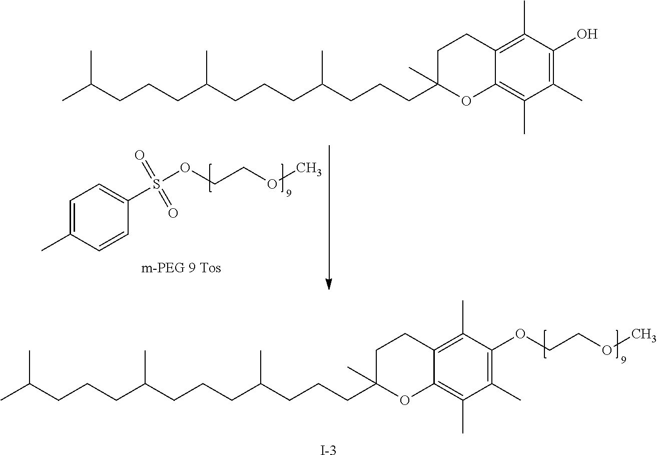

m-PEG 9 Tos was dissolved in anhydrous tetrahydrofuran. Sodium hydride (60% dispersion in mineral oil) was added portion wise, and the mixture was stirred for 1 hour at room temperature (RT). The tocopherol starting material was added portion wise and the reaction was stirred for 18 hours at RT. Upon completion the reaction was diluted with ethyl acetate. The organic layer was then washed with water, followed by a wash with brine. The organic layer was then dried with anhydrous magnesium sulfate. The crude material was purified by column chromatography to yield the desired product, compound I-3 as a yellow oil.

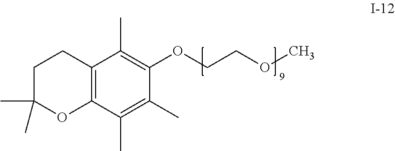

The structure of compound I-12 (TOC-dPEG9-OMe or TOC-240) was characterized by ¹H NMR (400 MHz, DMSO-d6): δ 3.51 (21H, s), 3.24 (3H, s), 2.08 (6H, d, J=8.0 Hz), 1.98 (3H, s), 1.74 (2H, t, J=5.7 Hz), 1.24 (6H, s).

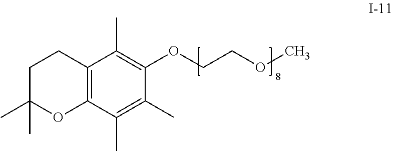

The structure of compound I-11 (TOC-dPEG8-OMe or LG-TOC-769 or TOC-769) was characterized by ¹H NMR (400 MHz, DMSO-d6): δ 3.50 (20H, s), 3.24 (3H, s), 2.08 (6H, d, J=9.9 Hz), 1.99 (3H, s), 1.73 (3H, t, J=6.2 Hz), 1.24 (6H, s).

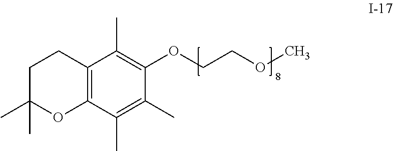

The structure of compound I-17 (TOC-dPEG8-OH or TOC-963) was characterized by ¹H NMR (400 MHz, CDCl₃) δ 3.669-3.563 (m, 22H), 2.527-2.490 (t, J=6.82 Hz, 2H), 2.101 (s, 3H), 2.063 (s, 3H), 1.998 (s, 3H), 1.726-1.689 (t, J=6.86 Hz, 2H), 1.592-1.518 (m, 3H), 1.383-1.294 (m, 3H), 1.211 (s, 6H), 0.928-0.890 (t, J=7.33 Hz, 4H).

Analogs are made by the same method, for example, using m-PEG-8 Tos to make PEG-8 analogs or using m-PEG-9 Tos to make PEG-9 analogs.

Example compounds that can be made by this method include, but are not limited to:

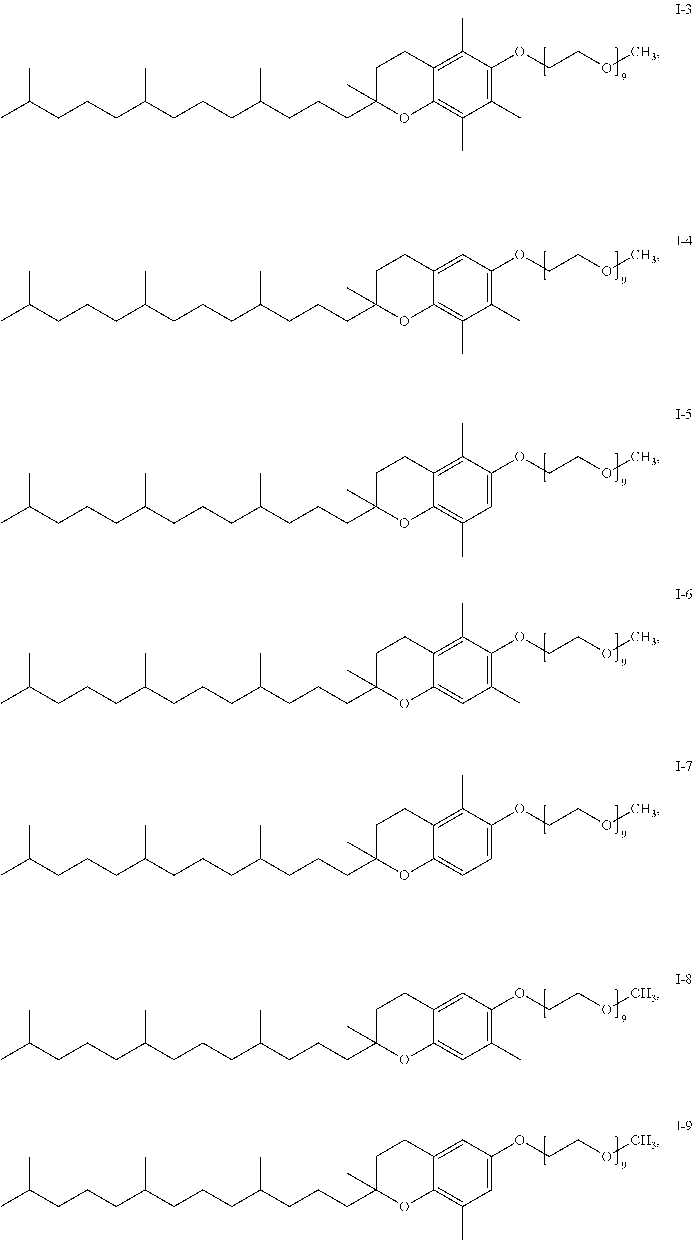

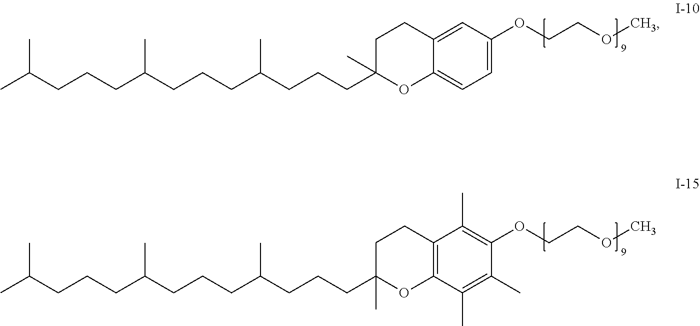

The structure of compound 1-15 was characterized by ¹H NMR (400 MHz, CDCl₃) δ 3.645-3.556 (m, 21H), 3.304 (s, 3H), 2.513-2.473 (t, J=6.75 Hz, 2H), 2.105 (s, 3H), 2.061 (s, 3H), 1.999 (s, 3H), 1.770-1.656 (m, 2H), 1.506-1.419 (m, 3H), 1.369-1.275 (m, 4H), 1.214-1.150 (m, 10H), 1.089-0.976 (m, 6H), 0.807-0.763 (m, 12H).

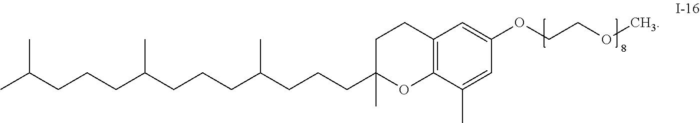

Similarly, additional analogs are made by the same method, for example, poly(ethylene glycol) methyl ether average molecular weight 550 and poly(ethylene glycol) methyl ether average molecular weight 750. For example, compounds I-13 and I-14.

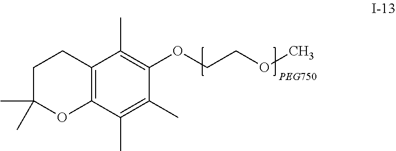

The structure of compound I-13 (TOC-PEG750-OMe or TOC-184) was characterized by ¹H NMR (400 MHz, DMSO-d6): δ 3.50 (58H, s), 3.24 (9H, s), 2.08 (6H, d, J=8.9 Hz), 1.99 (3H, s), 1.73 (3H, t, J=6.4 Hz), 1.24 (6H, s).

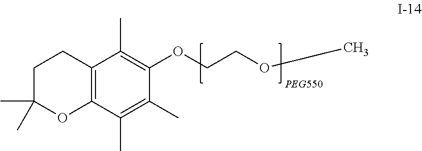

The structure of compound I-14 (TOC-PEG550-OMe or TOC-180) was characterized by ¹H NMR (400 MHz, DMSO-d6): δ 3.50 (37H, s), 3.24 (9H, s), 2.08 (6H, d, J=8.9 Hz), 1.99 (3H, s), 1.73 (3H, t, J=6.4 Hz), 1.24 (6H, s).

### Example 2

### General Procedure for Making Tos-PEG-TIPS Analogs

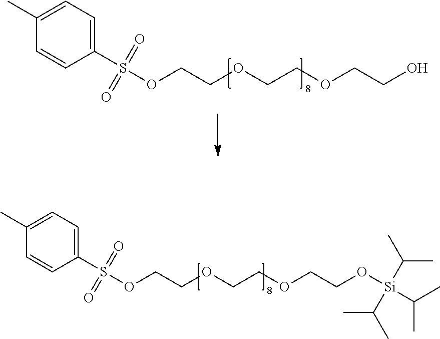

PEG 9 Tos was dissolved in anhydrous tetrahydrofuran. The mixture was cooled to 4 °C in an ice bath. Imidazole was added in one portion, followed by the dropwise addition of TIPS chloride. The reaction was then warmed to RT and stirred for 4 hours. Upon completion, the reaction was filtered and concentrated to dryness. The product was purified by column chromatography to yield the desired product.

### Example 3

### General Procedure to Make TIPS Protected PEG Analogs

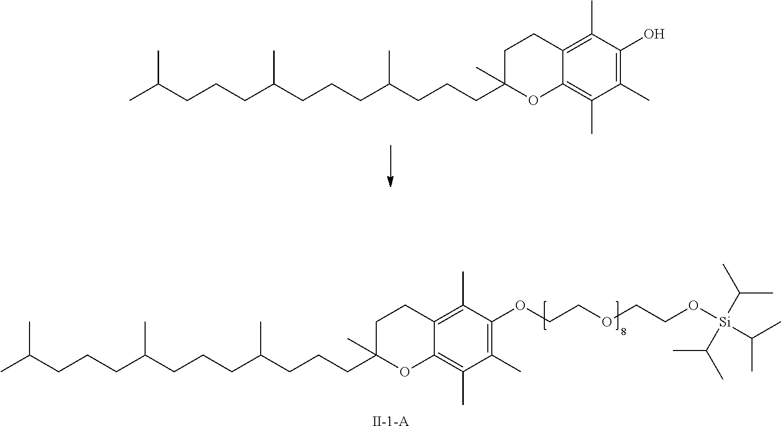

Tos-PEG 9-TIPS (for example, made according to Example 2) was dissolved in anhydrous tetrahydrofuran. Sodium hydride (60% dispersion in mineral oil) was added portion wise and the mixture was stirred for 1 hour at RT. The tocopherol starting material was added portion wise and the reaction was stirred for 18 hours at RT. Upon completion the reaction was diluted with ethyl acetate. The organic layer was then washed with water, followed by a wash with brine. The organic layer was then dried with anhydrous magnesium sulfate. The crude material was purified by column chromatography to yield the desired product as a yellow oil.

Example compounds that can be made by this method include, but are not limited to:

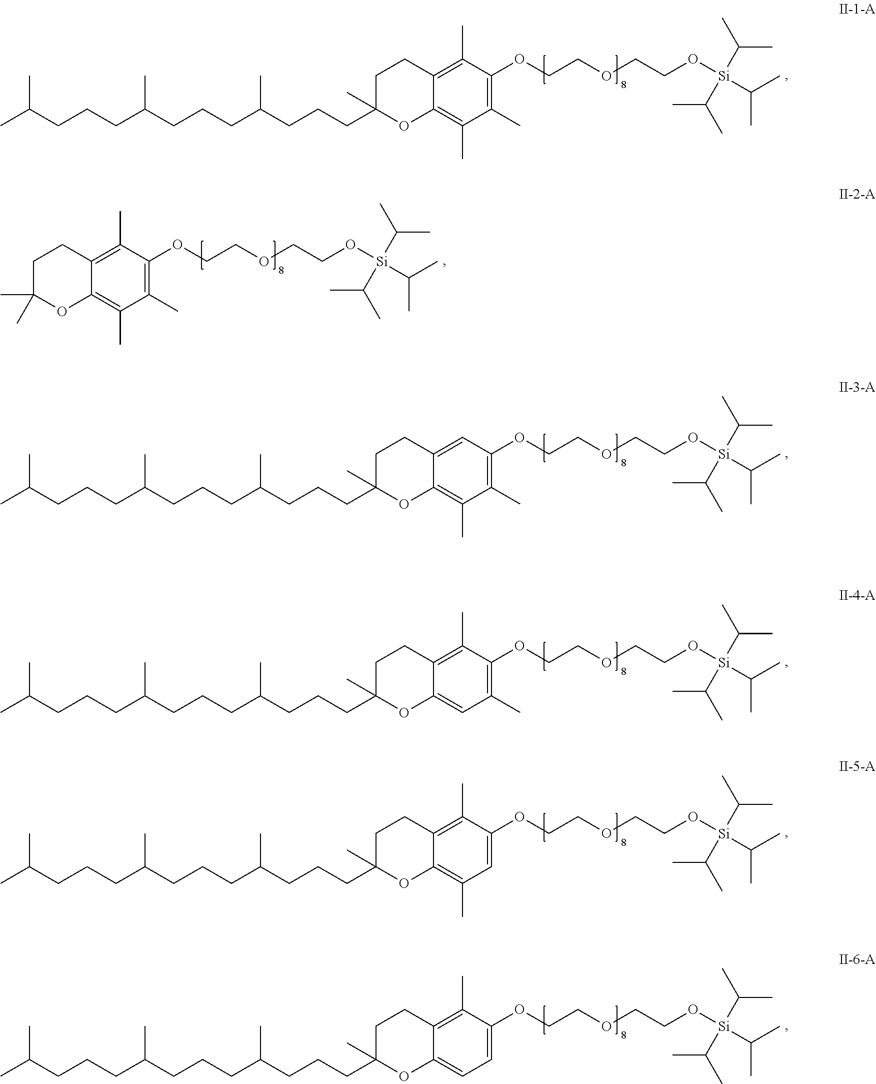

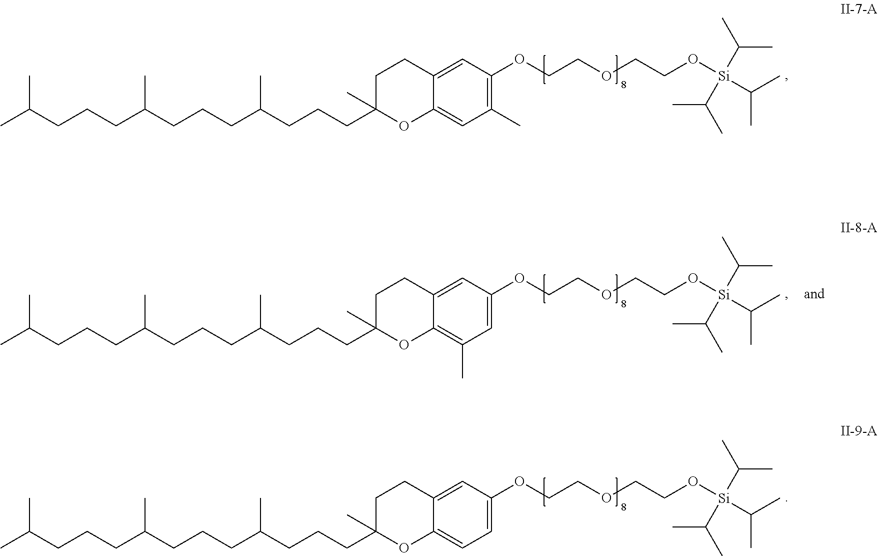

### Example 4

### General Procedure for Removing the TIPS Protecting Group

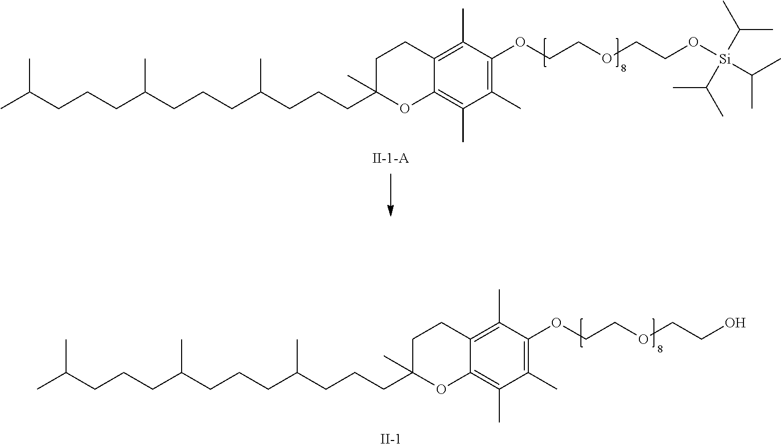

A Tocopherol-PEG-TIPS starting material (for example, made according to Example 3) was dissolved in tetrahydrofuran. Water was added in one portion, and the mixture was cooled to 4 °C in an ice bath. 1 M TBAF in tetrahydrofuran was added dropwise to the reaction, and the reaction was then warmed to RT and stirred for 3 hours. Once complete, the reaction was diluted with ethyl acetate. The organic layer was washed with brine and dried with anhydrous magnesium sulfate. The crude product was purified by column chromatography to yield the desired product.

Example compounds that can be made by this method include, but are not limited to:

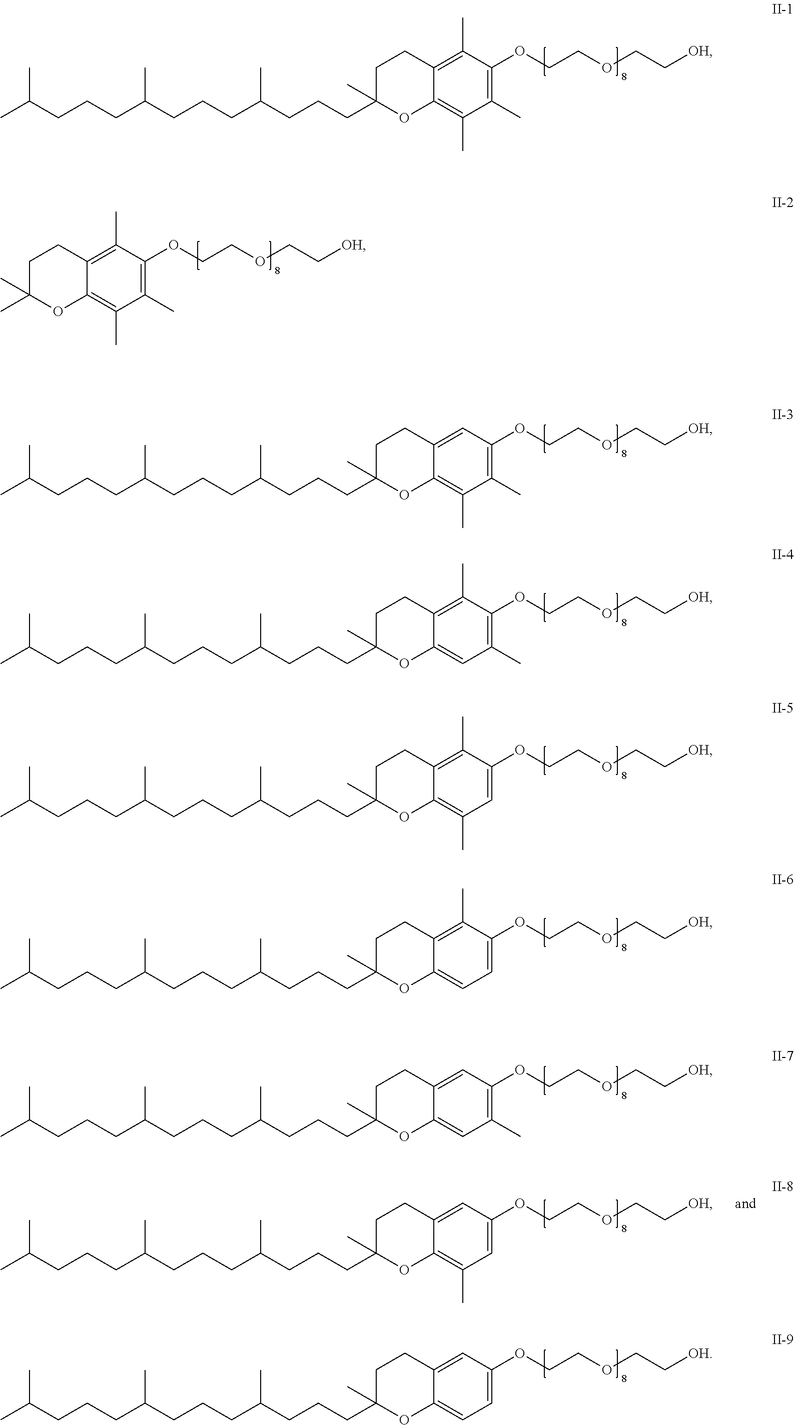

### Example 5

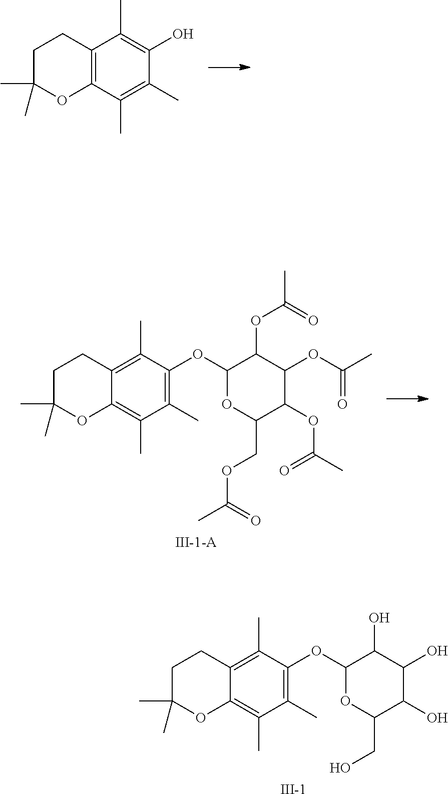

α-Acetobromogalactose was dissolved in anhydrous dichloromethane and oven dried 3 angstrom molecular sieves were added. The mixture was stirred for 20 minutes. The tocopherol starting material was added in one portion followed by the portionwise addition of silver carbonate. The reaction was stirred at room temperature for 18 hours, before being filtered through celite. The celite pad was washed with dichloromethane, and the filtrate was concentrated to dryness. The crude material was purified by column chromatography to yield the desired product.

The coupled tocopherol protected glucose material was dissolved in tetrahydrofuran. The mixture was cooled to 4 °C in an ice bath. A solution of lithium hydroxide in water was added was prepared in added dropwise to the reaction. The reaction was then warmed to room temperature and stirred for 18 hours. Once complete, the reaction was diluted with water and extracted with ethyl acetate. The organic layer was dried with anhydrous magnesium sulfate before being concentrated to dryness. The crude product was purified by column chromatography in order to yield the desired product.

The structure of compound III-1 (TOC-Glu or TOC-997) was characterized by ¹H NMR (400 MHz, CDCl₃) δ 4.987-1.974 (d, J=5.27 Hz, 1H), 3.762-3.725 (m, 1H), 3.668-3.645 (t, J=6.79 Hz, 1H), 3.622-3.604 (m, 1H), 3.588-3.548 (m, 2H), 3.533-3.488 (m, 1H), 2.623-2.588 (t, J=10.48 Hz, 2H), 2.176 (s, 3H), 2.161-2.135 (m, 4H), 2.055 (s, 3H), 1.825 (s, 3H), 1.811-1.775 (t, J=6.91 Hz, 2H), 1.274 (s, 6H).

Example compounds made by this method include, but are not limited to:

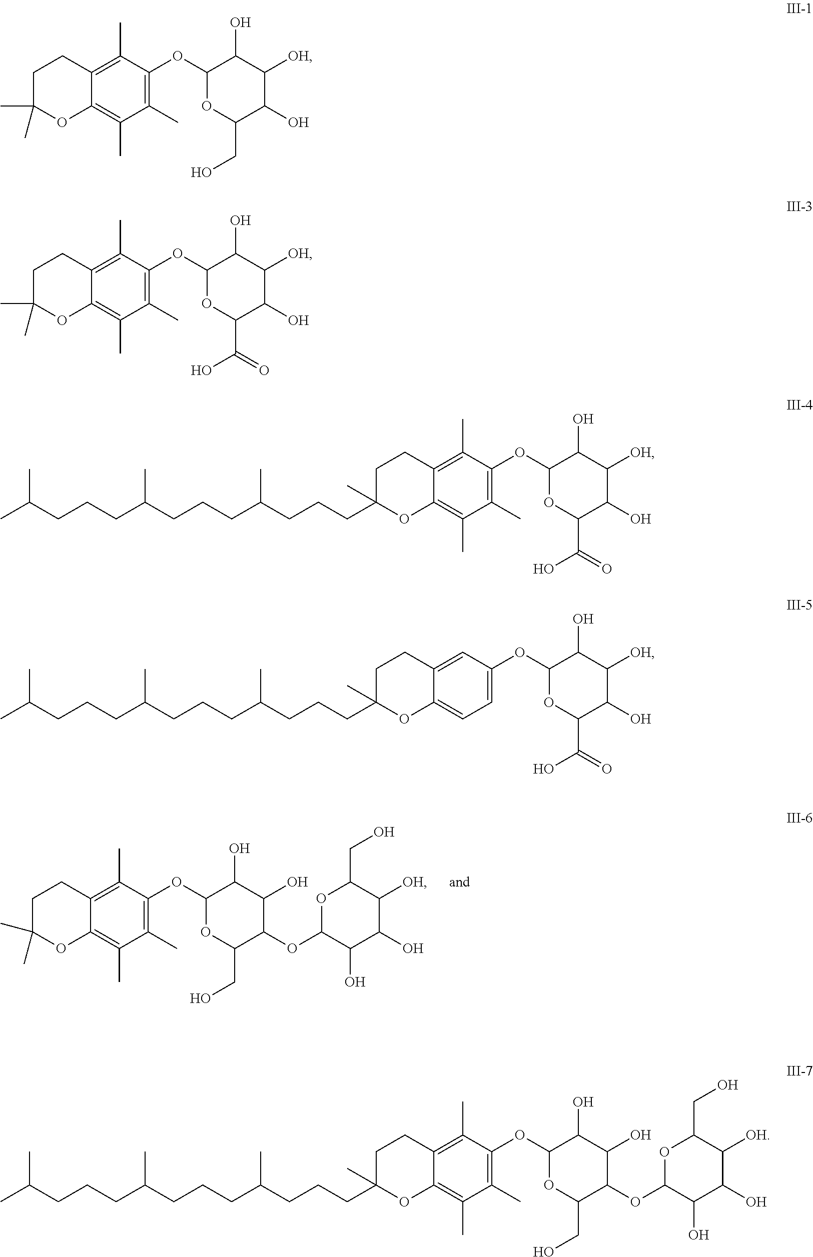

### Example 6

### General Procedure to Make Tocopherol Succinic Acid Derivatives

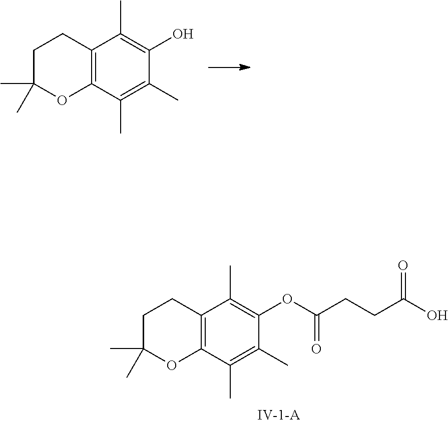

In a reaction flask, a tocopherol starting material, 4-DMAP and succinic anhydride was dissolved in anhydrous acetonitrile (MeCN) and stirred at room temperature. After completion of the reaction as judged by TLC, the reaction was concentrated to dryness then subsequently diluted with ethyl acetate, neutralized with saturated ammonium chloride and extracted with water. The organic layers were collected, combined and dried over Na₂SO₄, then filtered. The filtrate was concentrated to dryness. The crude product was purified by column chromatography to obtain the desired product as a solid white solid powder.

Example compounds made by this method include, but are not limited to:

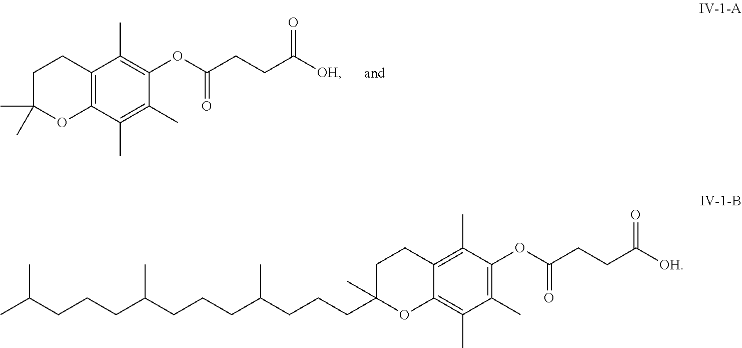 [SMILES: Cc1c(C)c2c(c(C)c1OC(=O)CCC(=O)O)CCC(C)(C)O2 ; Cc1c(C)c2c(c(C)c1OC(=O)CCC(=O)O)CCC(C)(CCCC(C)CCCC(C)CCCC(C)C)O2]

### Example 7

### General Procedure to Make Tocopherol PEG Analogs with a Succinic Acid Linker

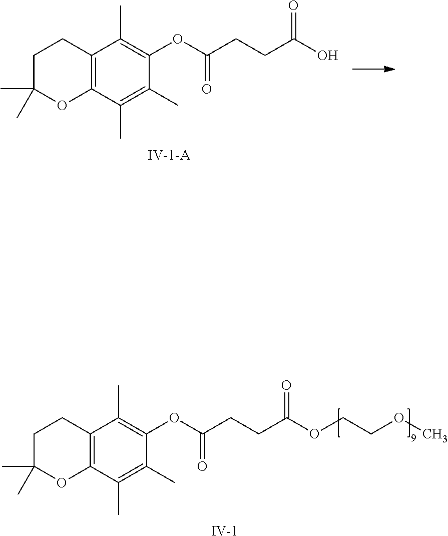

In a reaction flask, a tocopherol succinic acid derivative (for example, from Example 6), 4-DMAP and m-PEG 9 was dissolved in anhydrous MeCN and stirred at room temperature. N,N-diisopropylcarbodiimide was added dropwise. After completion of the reaction as judged by TLC, the mixture was concentrated to dryness and subsequently diluted in ethyl acetate and extracted with water. The organic layers were collected, combined, dried over Na₂SO₄ and filtered. The resulting filtrate was concentrated in vacuo and the crude product was isolated by column chromatography. The fractions were combined and dried in vacuo to obtain the desired product compound IV-1 (TOC-suc-dPEG9-OMe) as a colorless oil.

Example compounds made by this method but using alternative PEG analogs include, but are not limited to:

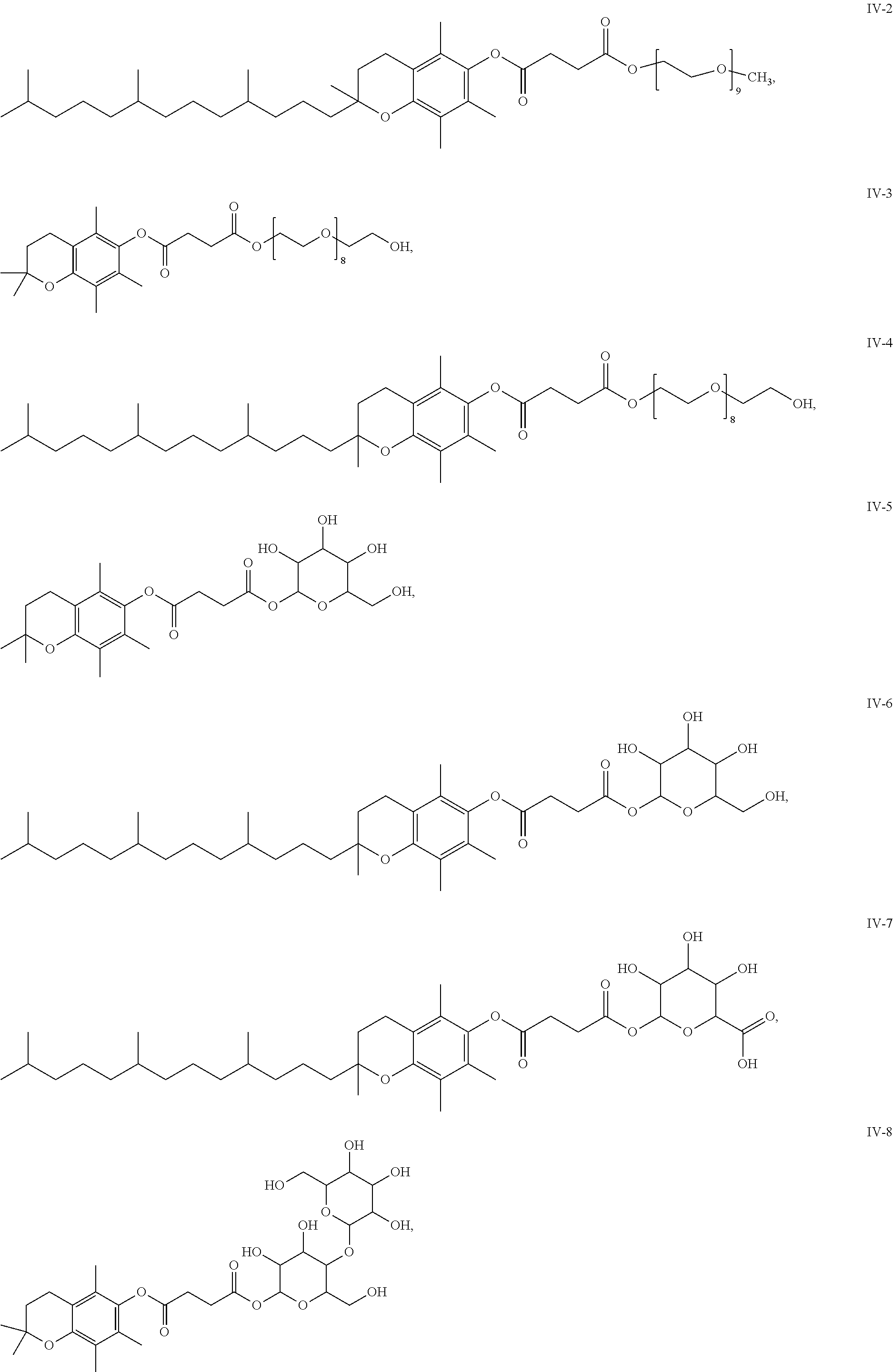

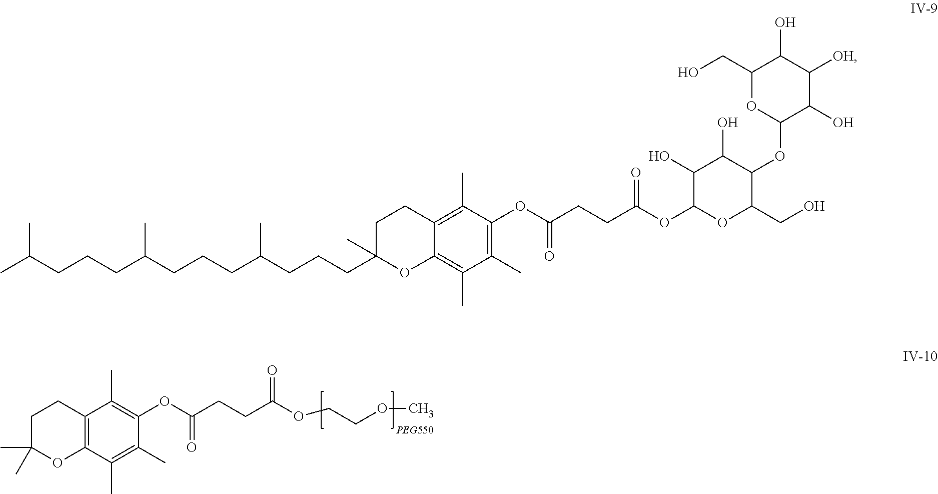

The structure of compound IV-10 (TOC-suc-PEG550-OMe or TOC-193) was characterized by ¹H NMR (400 MHz, CDCl₃): δ 4.20 (2H, t, J=5.4 Hz), 3.57 (48H, s), 3.31 (3H, s), 2.87 (2H, t, J=5.8 Hz), 2.73 (2H, t, J=6.6 Hz), 2.53 (2H, t, J=5.9 Hz), 2.01 (3H, s), 1.93 (6H, d, J=12.6 Hz), 1.71 (2H, t, J=5.4 Hz), 1.22 (6H, s).

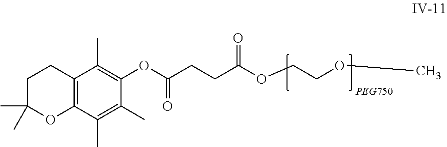

The structure of compound IV-11 (TOC-suc-PEG750-OMe or TOC-185) was characterized by ¹H NMR (400 MHz, CDCl₃): δ 4.29 (2H, t, J=4.2 Hz), 3.66 (68H, s), 3.40 (3H, s), 2.95 (2H, t, J=6.0 Hz), 2.82 (2H, t, J=6.2 Hz), 2.63 (2H, t, J=6.2 Hz), 2.10 (3H, s), 2.01 (6H, d, J=15.4 Hz), 1.31 (6H, s).

### Example 8

Frozen cell pellets (1×10⁷/pellet) were lysed with a test surfactant or a control surfactant using an immunoprecipitation (IP) lysis buffer recipe. The control surfactants were NP40 and EcoDurf-EH9 (EH9). Each surfactant experiment was run in duplicate. The cells were lysed with 0.5 mL of buffer and 1× protease inhibitor for 15 minutes on ice. The resultant mixture was centrifuged for 10 minutes at 17,000 g and 4 °C, and the resultant supernatant and pellets were collected.

The protein concentration of the supernatant was determined by BCA protein assay, subtracting the buffer only reading and the result was used for protein gels run at 10 g. SDS-page was run and the bands were assessed based on Coomassie quantitate with iBright™ software.

And western blots were run to assess some or all of the following:
HistoneH3 (15 kDa Anti-R^{b})—localized to nucleus, synthesized in the cytoplasm could be sign of nuclei damage during extraction
HSP70 (90 kDa Anti-M)—localized to cytoplasm
Cav-1 (17 kDa Anti-R^{b})—plasma membrane protein physically interacts with cholesterol with one integral domain
EGFR—(175 kDa Anti-R^{b})—transmembrane protein with one integral domain
ATP1A1—(100 kDa Anti-R^{b})—transmembrane protein with 10 integral domain
COXIV—(17 kDa Anti-R^{b})—inner mitochondrial transmembrane protein

Additionally for the test surfactants, the pellets were resuspended in 100 μL PBS and assessed under a microscope for intact nuclei.

### I. Assessment of Lysates of Cell Line A549 with Compound I-11 (LG-TOC-769)

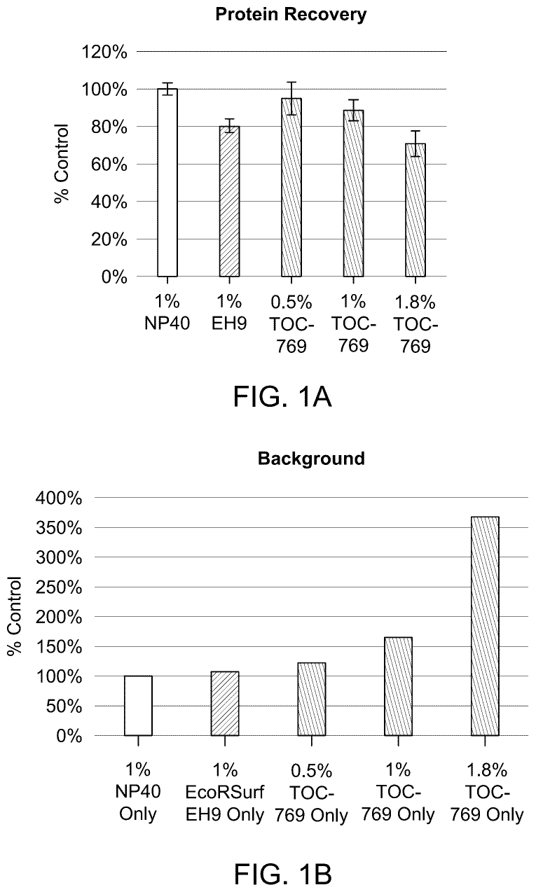

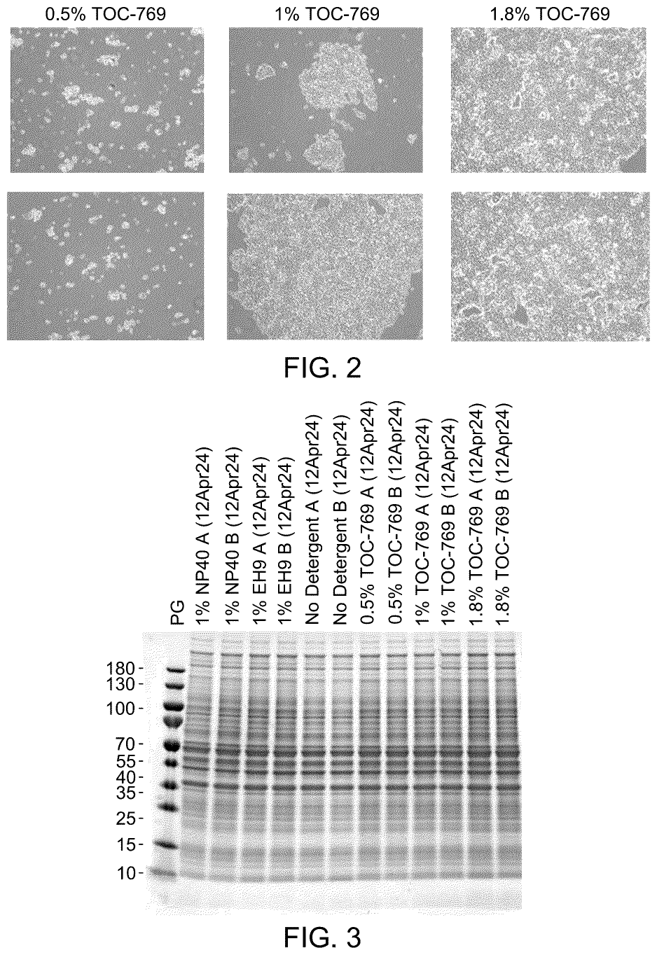

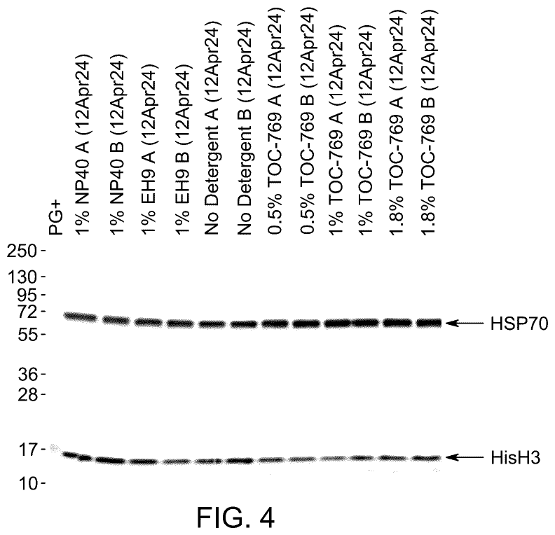

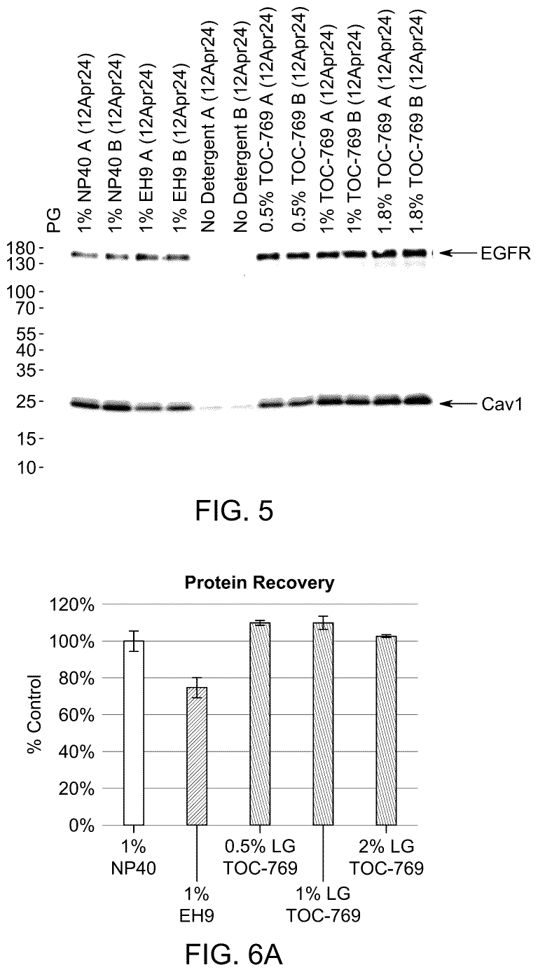

Compound I-11 was resuspended to 2% with DMSO added and used to lyse cell line A549. Compound I-11 demonstrated good protein recovery at lower doses, and some interference at higher doses (FIGS. 1A and 1B). Nuclei were present and dispersed at lower doses, but precipitant was seen at higher doses (FIG. 2). Comparable Coomassie bands were observed with SDS-page stained with Coomassie gel staining (FIG. 3). And good membrane protein recovery was observed (FIGS. 4 and 5).

### II. Assessment of Lysates of Cell Line HCT116 with Compound I-11 (LG-TOC-769)

Compound I-11 was resuspended to 2% and used to lyse cell line HCT 116 (Cell line HCT 116-CCL-247 available from ATCC).

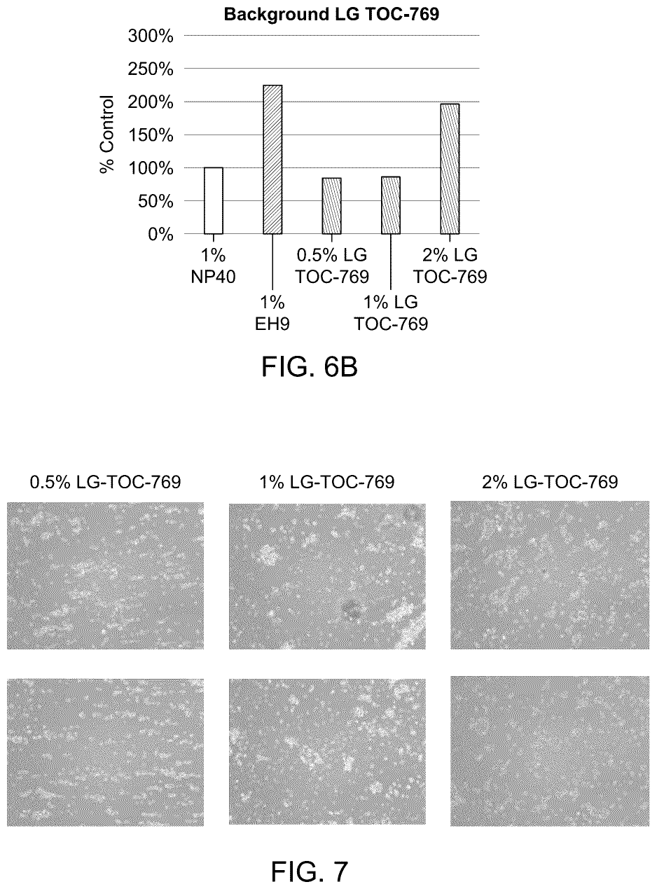

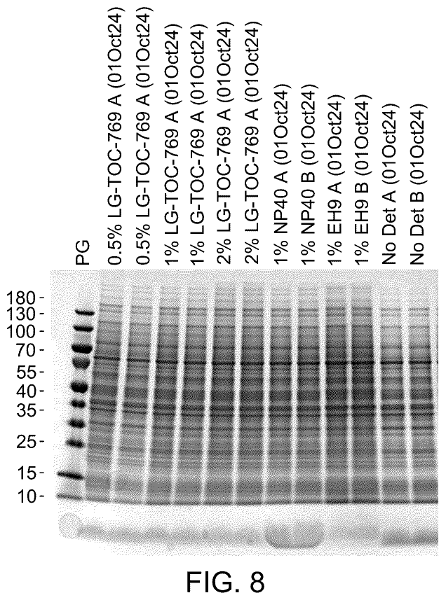

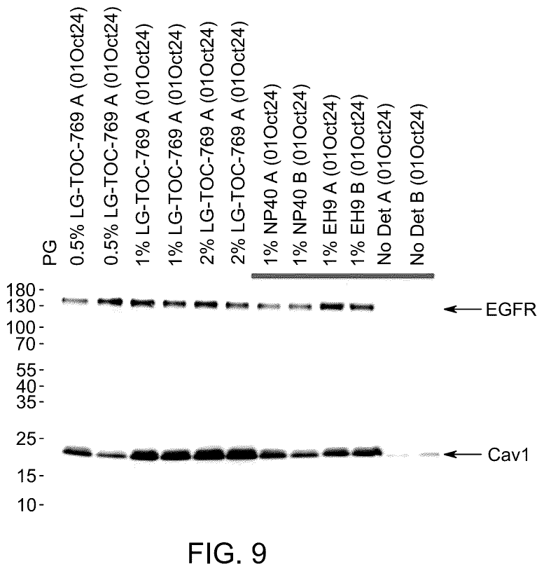

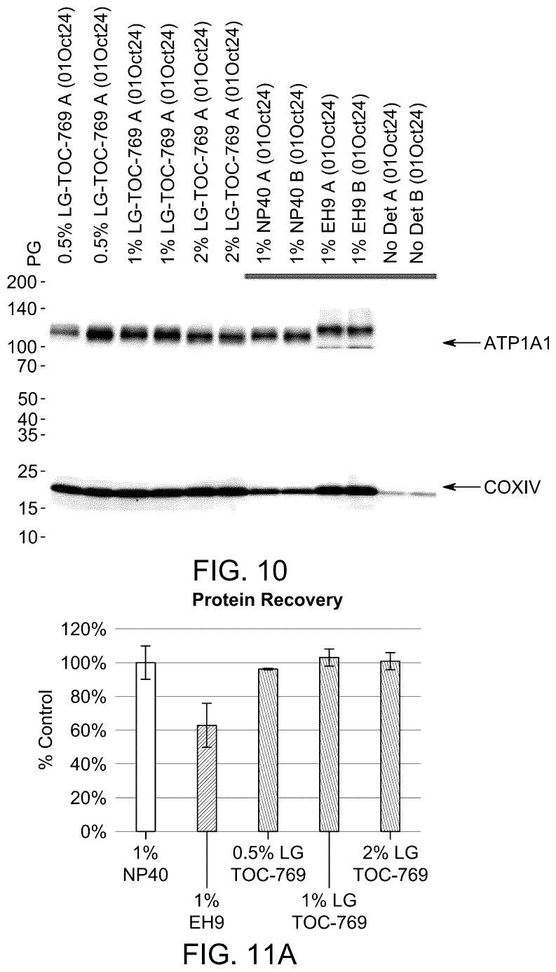

Compound I-11 demonstrated good protein recovery with no interference even at higher doses (FIGS. 6A and 6B). Nuclei were present and well dispersed (FIG. 7). Comparable Coomassie bands were observed with SDS-page stained with Coomassie gel staining (FIG. 8). And good membrane protein recovery was observed (FIGS. 9 and 10).

### III. Assessment of Lysates of Cell Line Expi293 with Compound I-11 (LG-TOC-769)

Compound I-11 was resuspended to 2% and used to lyse cell line Expi293.

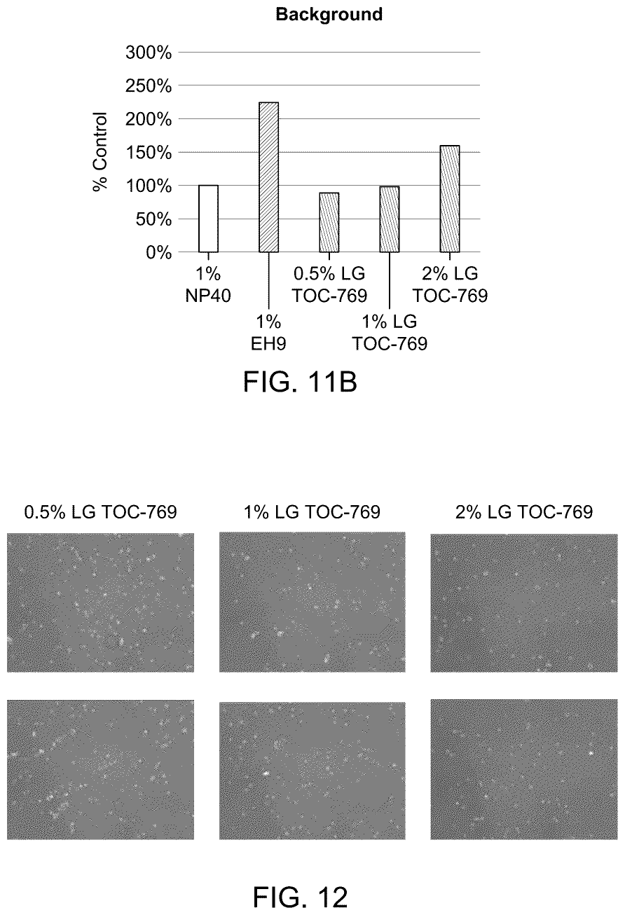

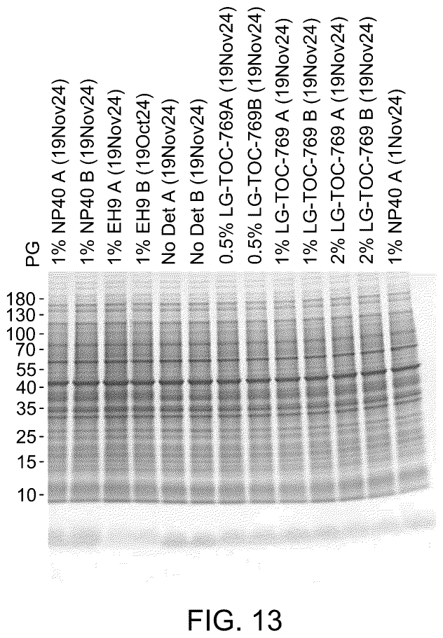

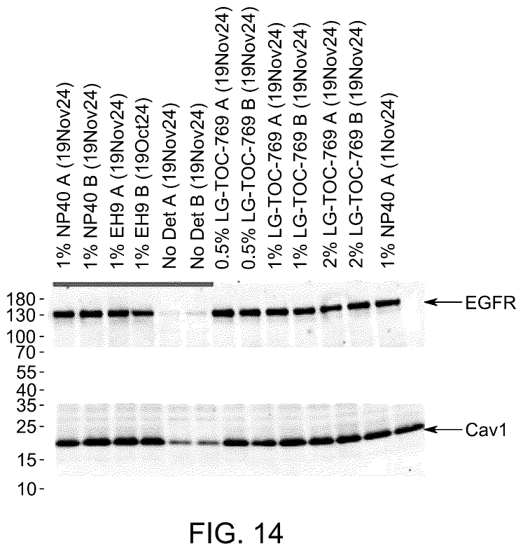

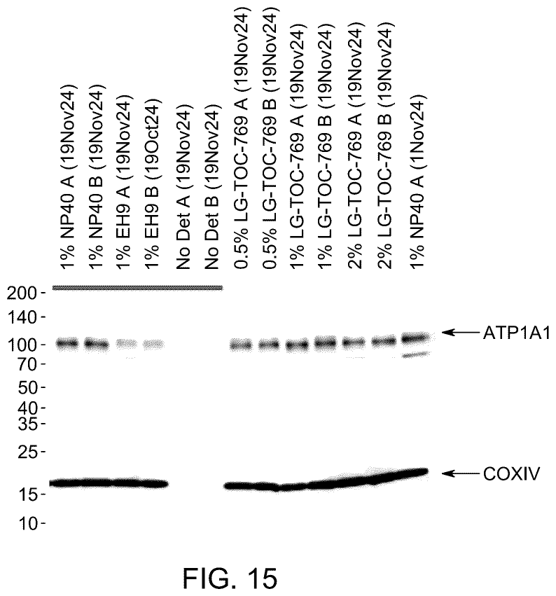

Compound I-11 demonstrated good protein recovery with no interference even at higher doses (FIGS. 11A and 11B). Nuclei were present and well dispersed (FIG. 12). Comparable Coomassie bands were observed with SDS-page stained with Coomassie gel staining (FIG. 13). And good membrane protein recovery was observed (FIGS. 14 and 15).

### Example 9

### Synthesis of Trolox-Based Compounds

### Step 1

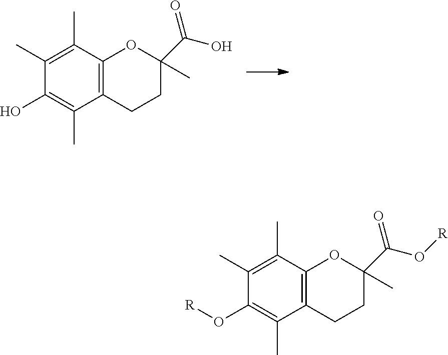

The following procedure can be used to add the following R groups to the molecule simply by substituting the alkyl bromide in the procedure
Straight alkyl chain up to 12 carbons in length
An aromatic ring by using benzyl bromide

Branched alkyl chains such as the following:

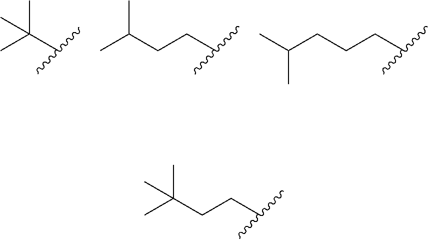

Procedure: All glassware and stir bar was oven dried before use. Trolox was dissolved in anhydrous dimethylformamide and the mixture was stirred at RT. Potassium carbonate was added in one portion and the reaction was heated to 100 °C for 30 min. The reaction was then cooled to 80 °C before the desired alkyl bromide was added dropwise to the reaction. The reaction was allowed to continue stirring at 80 °C for 6 hr until full consumption of the starting material was observed via TLC. Once complete, the reaction was cooled to RT before excess potassium carbonate was removed via filtration. The reaction was then concentrated to dryness before the residue was taken up in ethyl acetate. The organic layer was washed once with 1 M aqueous HCl, before being washed once with brine. The organic layer was then dried with anhydrous magnesium sulfate. The solvent was removed under reduced pressure and the crude material was purified via column chromatography to afford the desired di-substituted product.

### Step 2

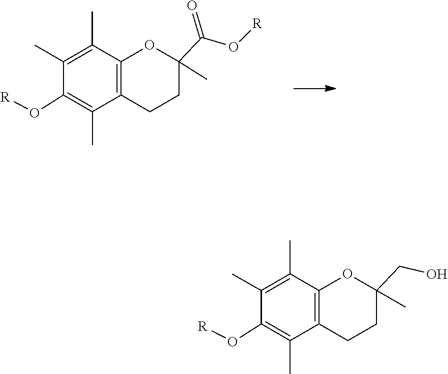

The following procedure can be used with any of the di-substituted compounds from the above procedure to selectively reduce the ester to a primary alcohol.

Procedure: All glassware and stir bar was oven dried before use. Anhydrous tetrahydrofuran was added to a flask followed by the portion wise addition of lithium aluminum hydride. The mixture was stirred and cooled to 4 °C. In a separate flask, a solution was prepared consisting of the starting material dissolved in anhydrous tetrahydrofuran. The solution containing the starting material was added dropwise to the reaction flask. The rate of addition was adjusted in order to minimize gas evolution. If too much gas was being produced, the rate of addition was slowed, and the rate of addition could be increased when gas evolution slowed. Once the addition was complete, the reaction was allowed to stir at 4 °C and the reaction was monitored by TLC. After 2 hr, full consumption of the starting material was observed via TLC. The reaction was quenched very slowly by the dropwise addition of methanol until gas evolution stopped. The solvent was then removed under reduced pressure and the residue was taken up in ethyl acetate. The organic layer was then washed water, followed by a wash with brine, before being dried with anhydrous magnesium sulfate. The organic layer was then concentrated to dryness. The crude material was purified via column chromatography to yield the desired product.

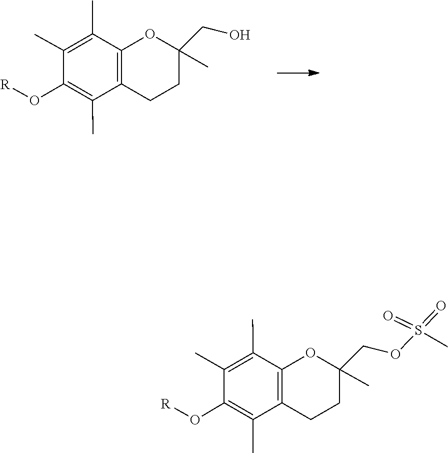

### Step 3

The following procedure can be used with any of the reduced compounds from the above procedure in order to add a mesylate leaving group.

Procedure: All glassware and stir bar was oven dried before use. The starting material was dissolved in anhydrous dichloromethane. The mixture was stirred and cooled to 4 °C. Triethylamine was added dropwise to the reaction, followed by the dropwise addition of a solution of methane sulfonyl chloride in anhydrous dichloromethane. The reaction was then warmed to RT and allowed to stir for 12 hr. Upon observing full consumption of the starting material via TLC, the reaction was quenched with a 50% aqueous brine solution. The organic layer was then washed with brine before being dried with anhydrous magnesium sulfate. The organic layer was then concentrated to dryness to yield the crude product. The crude material was carried forward to the next reaction without purification.

### Step 4

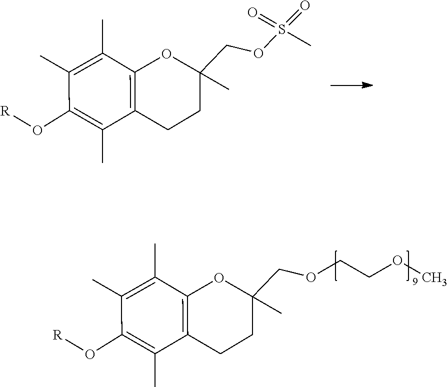

The following procedure can be used to add a PEG group to any of the mesylated compound synthesized from the above procedure

The procedure can be used to add any PEG length including discrete PEGs as well as PEG mixtures. Furthermore, this procedure can be used with capped m-PEGs as well as free hydroxy PEGs. Simply substitute the PEG in the procedure to achieve different analogs.

Procedure: All glassware and stir bar were oven dried before use. The desired PEG was dissolved in anhydrous tetrahydrofuran. Potassium tert-butoxide was added portion wise to the mixture, and the reaction was then cooled to 4 °C. In a separate flask, the mesylated starting material was dissolved in anhydrous tetrahydrofuran. The starting material solution was added dropwise to the reaction flask while stirring at 4 °C. Upon completion of the addition, the reaction was warmed to RT and allowed to stir for 18 hr. Once full consumption of the starting material was observed via TLC, the reaction was diluted with H₂O. 1 M HCL was then added dropwise until a pH of 6-7 was achieved. The reaction was then extracted with ethyl acetate. The organic layer was washed with brine before being dried with anhydrous magnesium sulfate. The solvent was removed under reduced pressure to yield the crude product. The crude material was purified via column chromatography in order to yield the desired product.

### Step 5

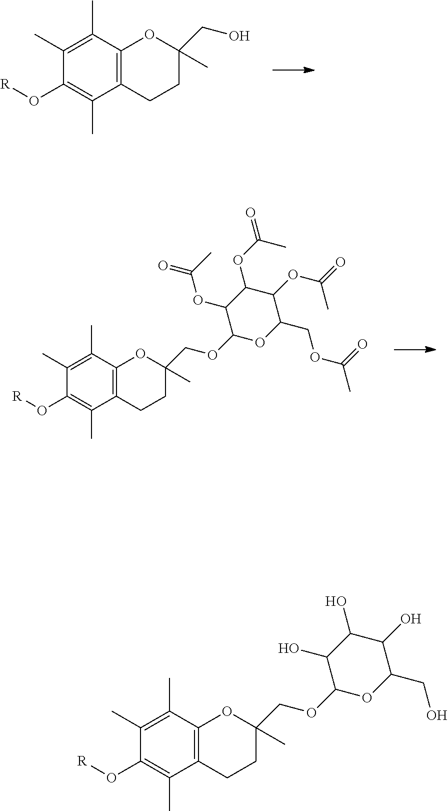

The following procedures are for first the coupling of the acetyl protected carbohydrate, and second for the acetyl deprotection to obtain the desired product

The coupling and deprotection can be conducted with any analog that has been reduced from the lithium aluminum hydride reaction

The following procedures can be conducted with any acetyl protected, bromine containing carbohydrate. This includes glucose, glucuronic acid, and carbohydrates containing multiple sugar units.

Some examples of final products are given below

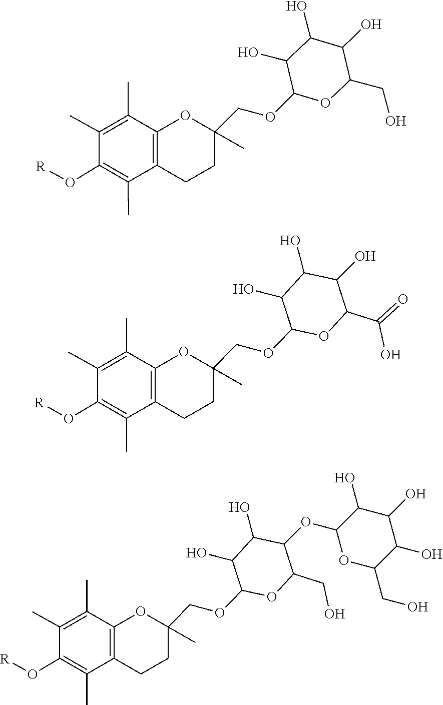

Procedures: α-Acetobromogalactose was dissolved in anhydrous dichloromethane and oven dried 3 angstrom molecular sieves were added. The mixture was stirred for 20 min. The starting material was added in one portion followed by the portion wise addition of silver carbonate. The reaction was stirred at RT for 18 hr. Before being filtered through celite. The celite pad was washed with dichloromethane, and the filtrate was concentrated to dryness. The crude material was purified by column chromatography to yield the desired product.

The carbohydrate coupled product was dissolved in tetrahydrofuran. The mixture was cooled to 4 °C in an ice bath. A solution of lithium hydroxide in water was prepared and added dropwise to the reaction. The reaction was then warmed to RT and stirred for 18 hr. Once complete, the reaction was diluted with water and extracted with ethyl acetate. The organic layer was dried with anhydrous magnesium sulfate before being concentrated to dryness. The crude product was purified by column chromatography in order to yield the desired product.

### Step 6

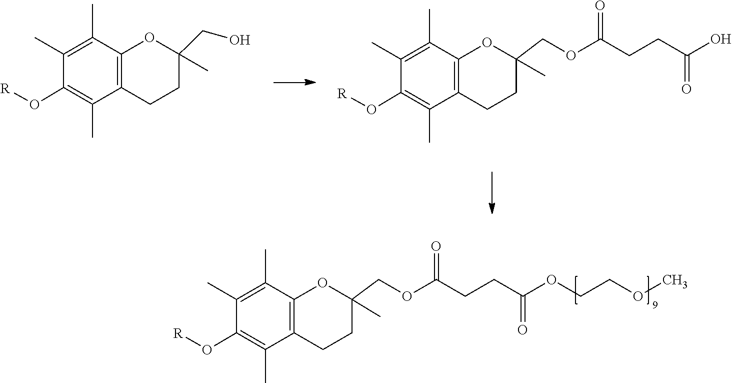

The following procedure are first for the coupling of a succinic acid linker onto the primary alcohol that was reduced in a previous reaction. This is followed by the procedure of the coupling of a PEG to the succinic acid

The procedure can be used to add any PEG length including discrete PEGs as well as PEG mixtures. Furthermore, this procedure can be used with capped m-PEGs as well as free hydroxy PEGs. Simply substitute the PEG in the procedure to achieve this different analogs.

Procedure: In a reaction flask, starting material, 4-DMAP and succinic anhydride was dissolved in anhydrous acetonitrile (MeCN) and stirred at room temperature. After completion of the reaction as judged by TLC, the reaction was concentrated to dryness then subsequently diluted with ethyl acetate, neutralized with saturated ammonium chloride and extracted with water. The organic layers were collected, combined and dried over Na₂SO₄, then filtered. The filtrate was concentrated to dryness. The crude product was purified by column chromatography to obtain the desired product.

In a reaction flask, the succinic acid derivative starting material, 4-DMAP and m-PEG 9 was dissolved in anhydrous MeCN and stirred at room temperature. N,N-diisopropylcarbodiimide was added dropwise. After completion of the reaction as judged by TLC, the mixture was concentrated to dryness and subsequently diluted in ethyl acetate and extracted with water. The organic layers were collected, combined, dried over Na₂SO₄ and filtered. The resulting filtrate was concentrated in vacuo and the crude product was isolated by column chromatography. The fractions were combined and dried in vacuo to obtain the desired product.

### Example 10

### In Silico Toxicology Predictions

Computational toxicology predictions were performed using a combination of QSAR (Quantitative Structure—Activity Relationship) and modeling platforms (including ECOSAR (Ecological Structure Activity Relationships) Class Program (Version 2-2), DEREK Nexus (Version 6.4.2), SARAH Nexus (Version 5.0.0), and ToxTree (Version 3.1.0-1851-1525442531402). The models assessed endpoints including: aquatic toxicity (fish, daphnia, algae); carcinogenicity; mutagenicity and genotoxicity; skin and eye irritation potential; endocrine disruption and reproductive toxicity alerts.

Comparative analyses were performed between select compounds of the current application, including Compound I-17 (TOC-dPEG8-OH (TOC-963)), and Compound IT-(TOC-Glu (TOC-997)) with results for Nonidet™ NP-40 (NP-40), and Triton™ X-100 (TX-100). Results were reported as No Alert/Low Concern, Equivocal/Moderate Concern, Positive Alert/High Concern, and Unknown.

Table 1 shows summary results for the in silico testing.

| | NP-40 | TX-100 | TOC-dPEG8-OH | TOC-Glu |
|---|---|---|---|---|
| ECOSAR: | Positive | Positive | No Alert/Low | Equivocal/ |
| Aquatic toxicity | Alert/High | Alert/High | Concern | Moderate |
| | Concern | Concern | | Concern |
| ToxTree: | No Alert/Low | No Alert/Low | No Alert/Low | No Alert/Low |
| Sensitization | Concern | Concern | Concern | Concern |
| ToxTree: | Unknown | Unknown | No Alert/Low | No Alert/Low |
| Eye irritation | | | Concern | Concern |
| ToxTree: | Positive | Positive | No Alert/Low | No Alert/Low |
| Skin irritation | Alert/High | Alert/High | Concern | Concern |
| | Concern | Concern | | |
| ToxTree: | No Alert/Low | No Alert/Low | No Alert/Low | No Alert/Low |
| Carcinogenicity | Concern | Concern | Concern | Concern |
| ToxTree: | No Alert/Low | No Alert/Low | No Alert/Low | No Alert/Low |
| Micronucleus | Concern | Concern | Concern | Concern |
| ToxTree: Ames | No Alert/Low | No Alert/Low | No Alert/Low | No Alert/Low |
| Genotoxicity | Concern | Concern | Concern | Concern |
| Nexus: ICHM7 | No Alert/Low | No Alert/Low | No Alert/Low | No Alert/Low |
| | Concern | Concern | Concern | Concern |
| Nexus: Sarah | No Alert/Low | No Alert/Low | No Alert/Low | No Alert/Low |
| | Concern | Concern | Concern | Concern |
| Nexus: Derek | Positive | Positive | Positive | No Alert/Low |
| (Other Alerts) | Alert/High | Alert/High | Alert/High | Concern |
| | Concern | Concern | Concern | |
| Nexus: Derek | No Alert/Low | No Alert/Low | No Alert/Low | No Alert/Low |
| (Mutagenicity) | Concern | Concern | Concern | Concern |

Table 1 shows the results from ECOSAR (aquatic toxicity), ToxTree (sensitization, eye irritation, skin irritation, carcinogenicity, micronucleus, Ames genotoxicty), and NEXUS (ICHM7, Sarah, Derek). Results are reported as No Alert/Low Concern, Equivocal/Moderate Concern, Positive Alert/High Concern, and Unknown.

The in silico results predict that compounds of Formula I have improved toxicological and environmental safety profiles as compared to Triton™ X-100 and Nonidet™ NP-40, particularly with respect to aquatic toxicity. These findings support their potential as safer, more environmentally-friendly, regulatory-compliant substitutes for non-ionic surfactants in life science, industrial, and diagnostic applications.

### Example 11

### Fish Acute Toxicity Test (OECD 236)

The acute aquatic toxicity of certain compounds disclosed herein on freshwater fish are evaluated under controlled laboratory conditions in accordance with OECD Test Guideline 236 (Fish Embryo Acute Toxicity Test). This study is designed to determine the maximum tolerable dose (MTD) which does not cause overt toxicity, and the concentration that results in 50% lethality (LC50).

To test the acute aquatic toxicity Danio rerio (zebrafish) are used as a representative freshwater species. The test system and environmental parameters are maintained as follows: zebrafish embryos are fertilized. At three hours post-fertilization (hpf), one embryo is placed in each well of a pre-conditioned 24-well plates. Liquid is gently aspirated from the wells of the 24-well plate and replaced by 2.0 ml of test solution, consisting of solutions containing compounds of Formula I in varying concentrations (0.1, 0.3, 1, and 3 mg/L (ppm)). Additional wells are similarly prepared using by-products of Compounds of Formula I, TX-100 and NP-40. Plates are sealed and maintained at a temperature of 26 °C. Initial assessment of the embryos occurs at 24 hpf when the presence of dead embryos is recorded, observations recorded, and the plates resealed. This is repeated at 24-hour intervals up to 96 hpf Phenotypical data are entered as 1 or 0 (1=abnormal, 0=normal) for the following: mortality, somites, detachment of the tail-bud from the yolk sac, heartbeat, cardiac edema and hatching. Overt deformities in other areas are noted. LC₅₀ values are estimated at 96 hours.

Based on in silico modeling, the 96-hour LC₅₀ for compounds of Formula I indicate low acute aquatic toxicity under GHS and REACH classification schemes as compared to Triton™ X-100 and Nonidet™ NP-40.

The results of this study corroborate in silico toxicity predictions, demonstrating that compounds of Formula I present minimal risk to aquatic organisms and may be considered environmentally safer alternatives to Triton™ X-100 and Nonidet™ NP-40

### Example 12

### Fish Acute Toxicity Test (Daphnia magna)

Comparative acute aquatic toxicity of projected degradation by-product of Formula I was compared to the known degradation by-products Triton™ X-100 and Tergitol™ NP-40. Daphnia magna was selected as a representative freshwater invertebrate model organism to assess acute toxicity, using DAPHTOXKIT F test kit by Microbiotests, Inc.

Dormant eggs of Daphnia magna were rinsed to remove storage media and transferred to a hatching petri dish in 15 ml pre-aerated standard freshwater. Petri dish was covered and incubated for 72 h at 20-22 °C to hatch.

Solutions of projected potential by-product of Formula I:

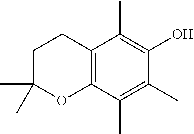

and the known by-products of Triton™ X-100:

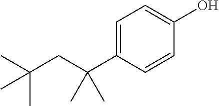

### and Tergitol™ NP-40:

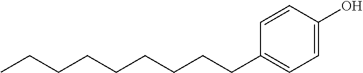 [SMILES: CCCCCCCCCc1ccc(O)cc1]

were prepared in the following manner: each by-product was diluted to 1% in methanol and then diluted to 0.001% in twice distilled H2O, to reach a concentration of 10 mg/L. The solutions were further diluted to 2.0 mgL, 3.0 mg/L and 5.0 mg/L for each by-product. Solutions of 2.0 mgL, 3.0 mg/L and 5.0 mg/L methanol were prepared.

A multiwell plate was labeled and 10 mL saltwater added to a well as control; an additional well was prepared using 10 mL of 2.0 mg/L K₂Cr₂O₇. 10 mL of each concentration of methanol or TX-100, NP-40 or Formula I by-product was added into separate wells. Next, 5 Daphnia neonates were transferred into each test well. The plate was covered tightly and incubated at 20 °C in darkness. Number of immobilized (including dead) neonates were recorded at 24 h and 48 h. Percentage immobilized neonates were calculated (Table 2).

TABLE 2

| | 24 HR | 48 HR |
|---|---|---|
| Control | 0% | 5% |
| 2.0 mg/L K₂Cr₂O₇ | 100% | 100% |
| 2.0 mg/L Meth | 0% | 0% |
| 3.0 mg/L Meth | 0% | 0% |
| 5.0 mg/L Meth | 0% | 0% |
| 2.0 mg/L Triton ™ X-100 By-product | 5% | 15% |
| 3.0 mg/L Triton ™ X-100 By-product | 45% | 55% |
| 5.0 mg/L Triton ™ X-100 By-product | 95% | 100% |
| 2.0 mg/L Nonidet ™ NP-40 By-product | 15% | 55% |
| 3.0 mg/L Nonidet ™ NP-40 By-product | 100% | 100% |
| 5.0 mg/L Nonidet ™ NP-40 By-product | 100% | 100% |
| 2.0 mg/L Formula I By-product | 5% | 5% |
| 3.0 mg/L Formula I By-product | 5% | 5% |
| 5.0 mg/L Formula I By-product | 0% | 0% |

Table 2 shows Daphnia study results with percentage of fish immobilized after 24 and 48 hours in saltwater control, K₂Cr₂O₇; methanol at 2.0, 3.0, or 5.0 mg/L; and by-products of Triton™ X-100, Nonidet™ NP-40, or compounds of this disclosure at 2.0, 3.0, and 5.0 mg/L.

Results show that the by-product of Formula I showed significantly lower aquatic less toxicity than the K₂Cr₂O₇ control, Triton™ X-100 by-product, and Nonidet™ NP-40 by-product. Immobilization did not increase significantly with higher doses of Formula I by-product, while significantly higher dose-dependent immobilization was observed with the by-products of Triton™ X-100 and Nonidet™ NP-40. The by-product of Formula I did not reach EC₅₀ (the concentration at which 50% of the organisms are immobilized) at 5.0 mg/L. Similar tests are performed on the individual compounds to further demonstrate similar improved toxicity profile. The results of this study demonstrate that compounds of this disclosure are environmentally safer alternatives to Triton™ X-100 and Nonidet™ NP-40.

### Example 13

### Biodegradability (OECD 301B)

In this example, compounds of this disclosure are tested according to OECD 301B to determine biodegradability. The compounds of this disclosure are tested, and biodegradability results are calculated, according to acceptable test conditions under OECD 301B. A compound is determined to be “readily biodegradable” if it achieves a 60% biodegradability result, which is equivalent to 60% of ThOD (theoretical oxygen demand) or ThCO₂ (theoretical carbon dioxide) production within a ten-day window during a 28-day test.

Compounds of this disclosure are anticipated to achieve a higher percentage of biodegradability than either Triton™ X-100 or Nonidet™ NP-40.

In view of the many possible aspects to which the principles of the disclosure may be applied, it should be recognized that the illustrated aspects are only preferred examples of the disclosure and should not be taken as limiting the scope of the disclosure. Rather, the scope of the disclosure is defined by the following claims. We therefore claim as the disclosure all that comes within the scope and spirit of these claims.

1. A compound having a structure according to Formula I 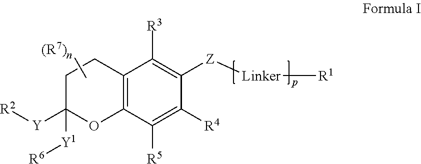
wherein:
R¹ is —([(CH₂)ᵥ]—O)_{z}—R^{c},
each y independently is 2 or 3:
z is from 4 to 25; and
R^{c} is H or C₁₋₄alkyl;
R² is methyl or C₂₋₂₅alkyl;
each of R³, R⁴ and R⁵ independently is H, C₁₋₆alkyl, —OC₁₋₆alkyl, C₃₋₆cycloalkyl, —OC₃₋₆cycloalkyl, 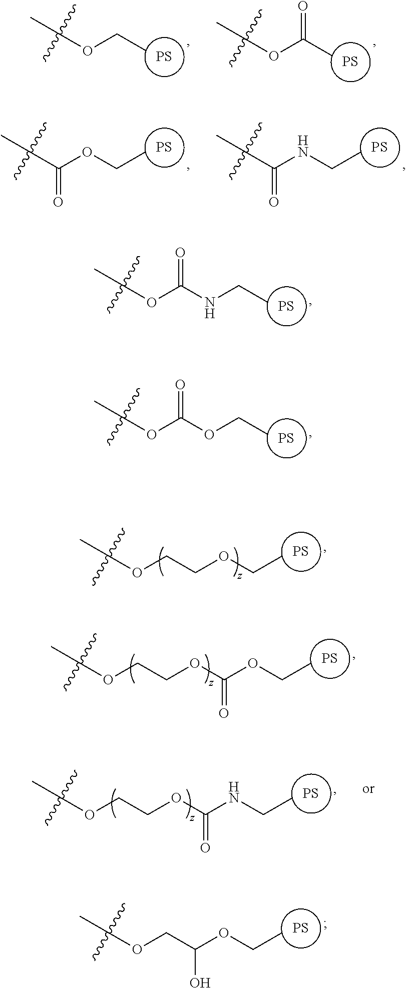
PS is a polystyrene bead;
R⁶ is H or C₁₋₃₀alkyl;
R⁷ is C₁₋₆alkyl;
n is 0, 1, 2, 3, or 4;
Y is a bond, O, or N(R^{a});
Y¹ is a bond, O, or N(R^{a});
Z is O, N(R^{a}), C(═O), (C═O)N(R^{a}), N(R^{a})C(═O), or a bond;
Linker is —(CH₂)ₓO—, —(CH₂)ₓC(═O)N—, or —(CH₂)ₓNC(═O)—;
x is 1, 2, 3, or 4;
p is 0 or 1; and
each R^{a} independently is H, C₁₋₄alkyl.

2. The compound of claim 1, wherein R³, R⁴, R⁵, are independently —O—C₁₋₆alkyl, —O—C₃₋₆cycloalkyl,

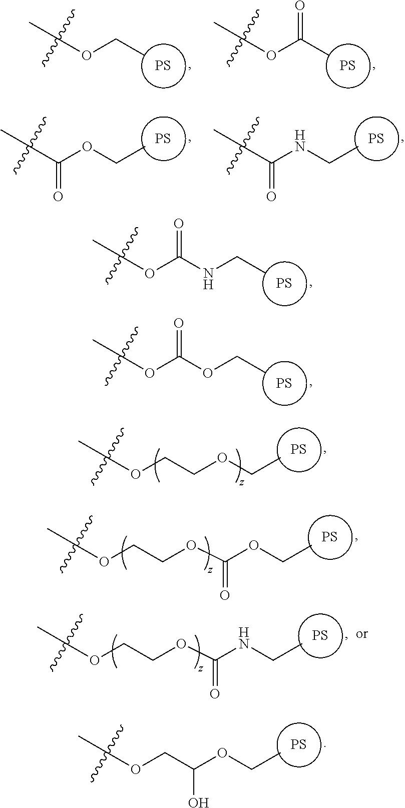

3. The compound of claim 1, wherein R¹ is

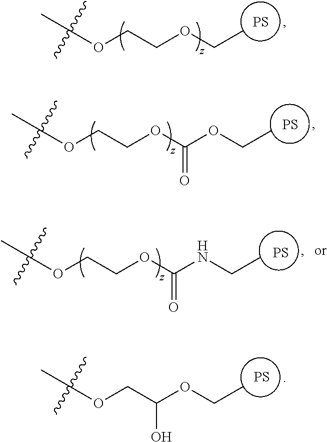

4. The compound of claim 1, wherein R² is C₄₋₂₀alkyl.

5. The compound of claim 1, wherein R² is

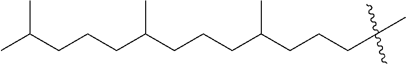 [SMILES: *CCCC(C)CCCC(C)CCCC(C)C]

6. The compound of claim 1, wherein R⁶ is methyl.

7. The compound of claim 1, wherein the compound has a structure according to Formula II or Formula III

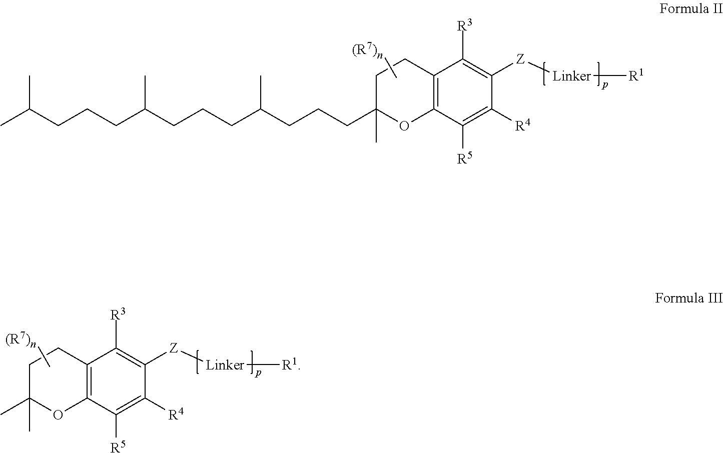

8. The compound of claim 1, wherein the compound has a structure according to Formulas IIa, IIb, IIc, IId, IIIa, IIb, IIc, or IIId

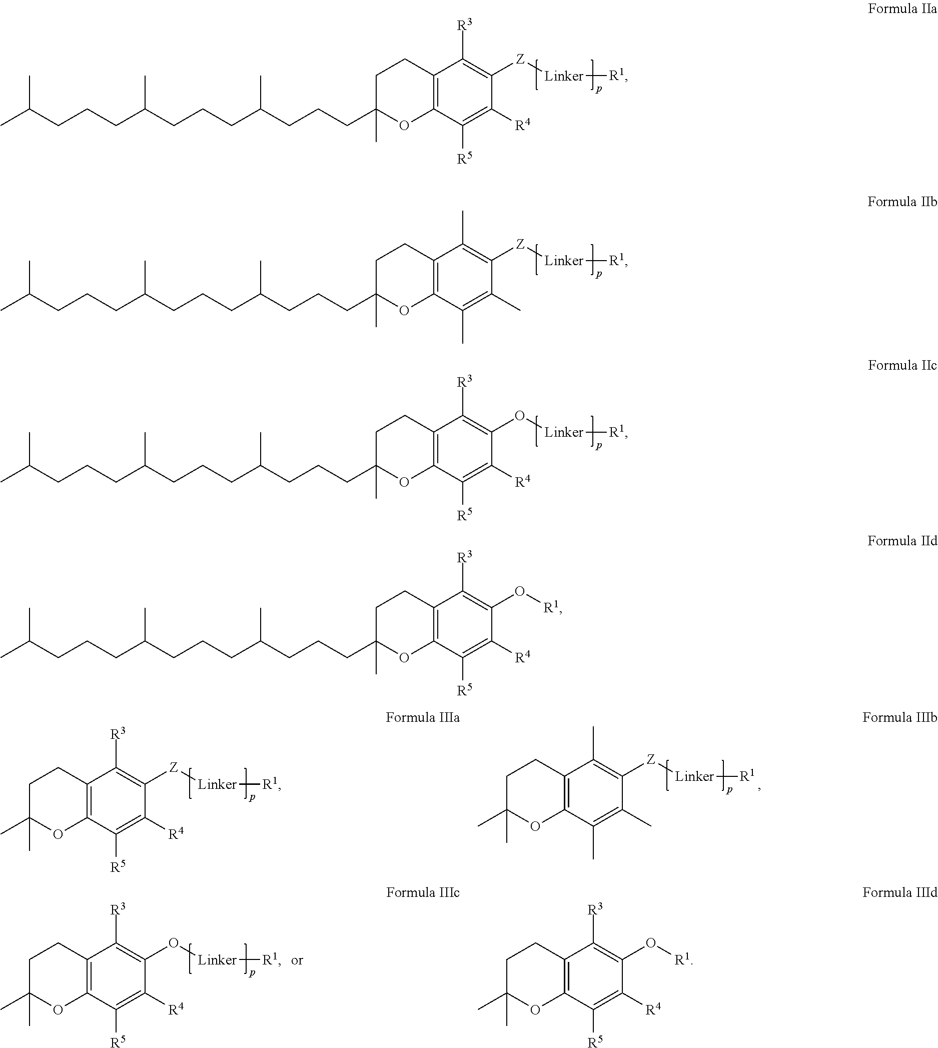

9. The compound according to claim 1, wherein R¹ is a polyalkylene oxide group.

10. The compound according to claim 9, wherein the polyalkylene oxide group is polyethylene glycol (PEG), polypropylene glycol (PPG), or a combination thereof, optionally terminated in an alkyl group.

11. (canceled)

12. The compound of claim 1, wherein R¹ is a PEG or PPG group having a mass average molecular weight ranging from 500 to 800 daltons.

13. The compound of claim 1, wherein when R¹ is a heterocycloaliphatic group, the heterocycloaliphatic group is a 5- or 6-membered oxygen-containing heterocycloaliphatic group.

14. The compound of claim 13, wherein the heterocycloaliphatic group is derived from a sugar moiety.

15. The compound of claim 14, wherein
R¹ is 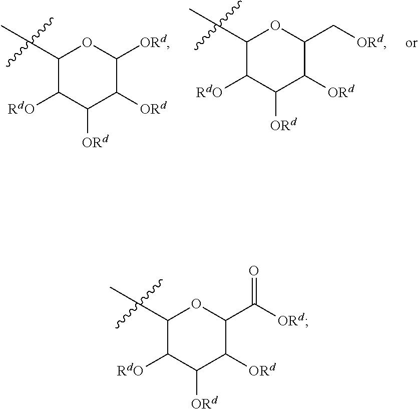
each R^{d} independently is H, a polyalkylene oxide group,

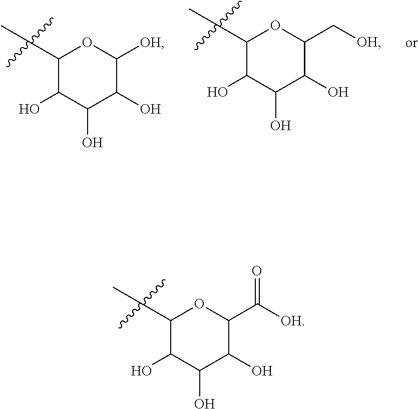 [SMILES: *C1OC(O)C(O)C(O)C1O ; *C1OC(CO)C(O)C(O)C1O ; *C1OC(C(=O)O)C(O)C(O)C1O]

16. The compound of claim 15, wherein the polyalkylene oxide group is polyethylene glycol (PEG), polypropylene glycol (PPG), or a combination thereof, optionally terminated in an alkyl group.

17. The compound of claim 1, wherein R¹ and R² are selected to provide a hydrophilic-lipophilic balance (“HLB”) value ranging from 10 to 18.

18. The compound of claim 1, selected from

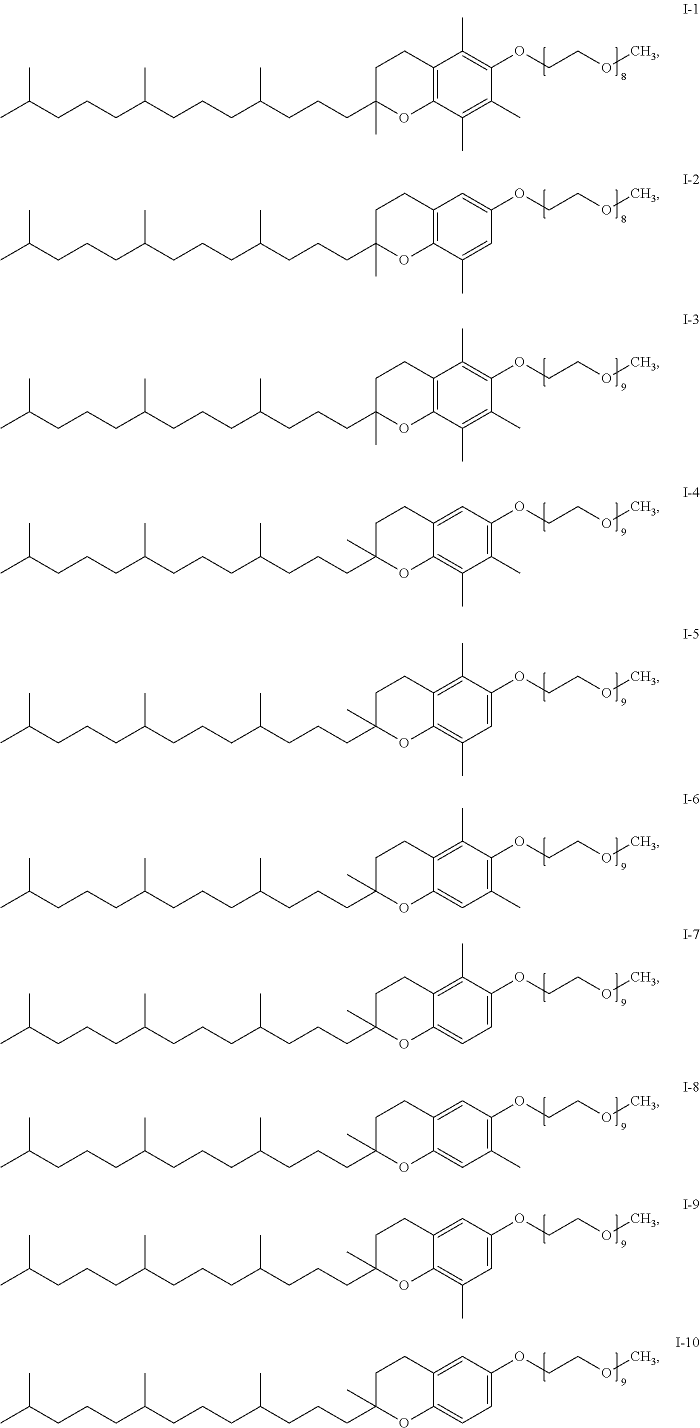

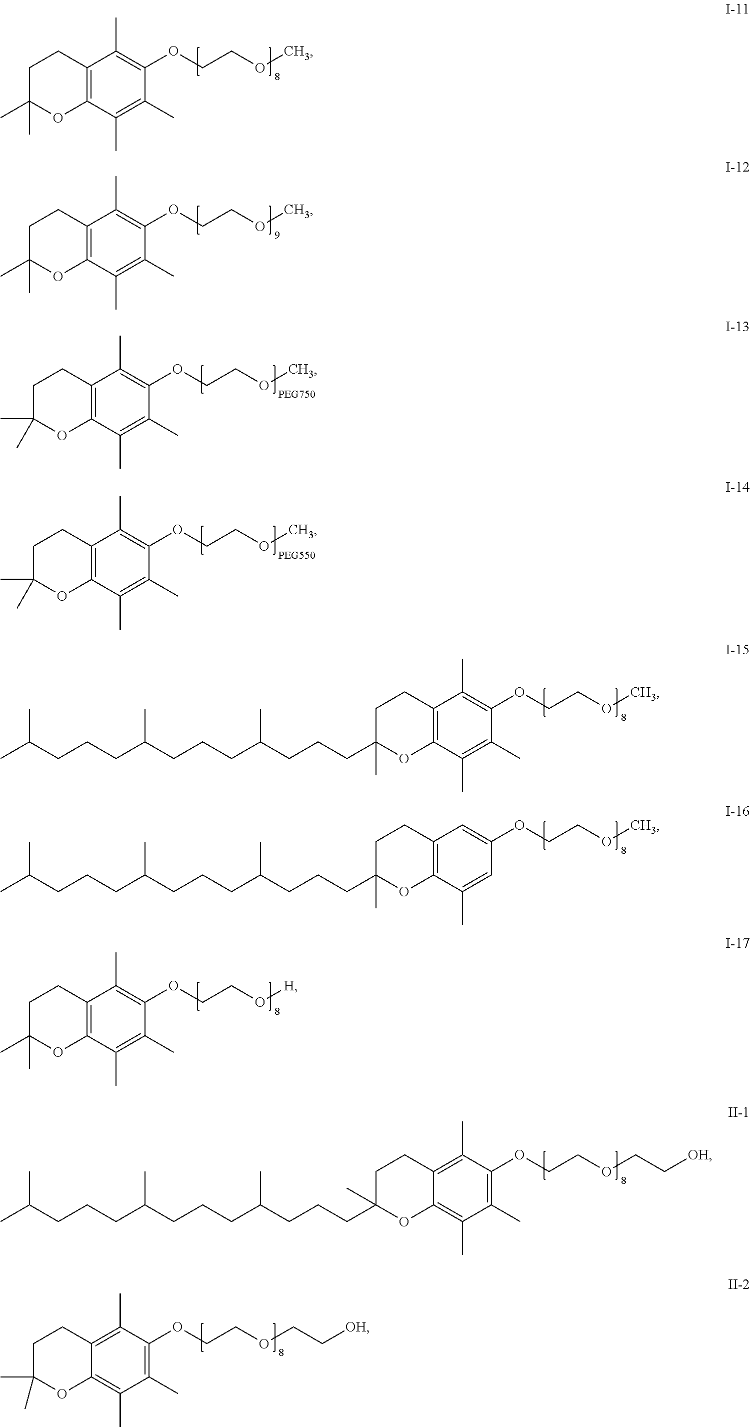

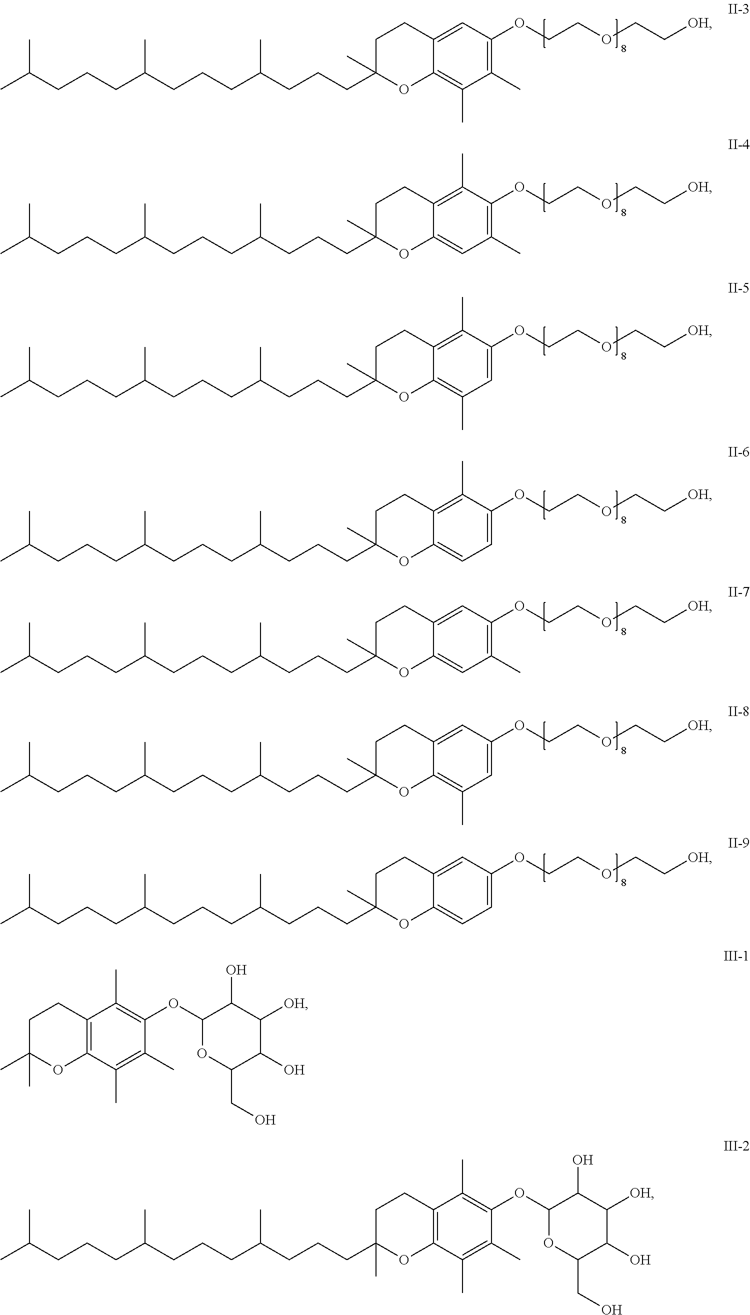

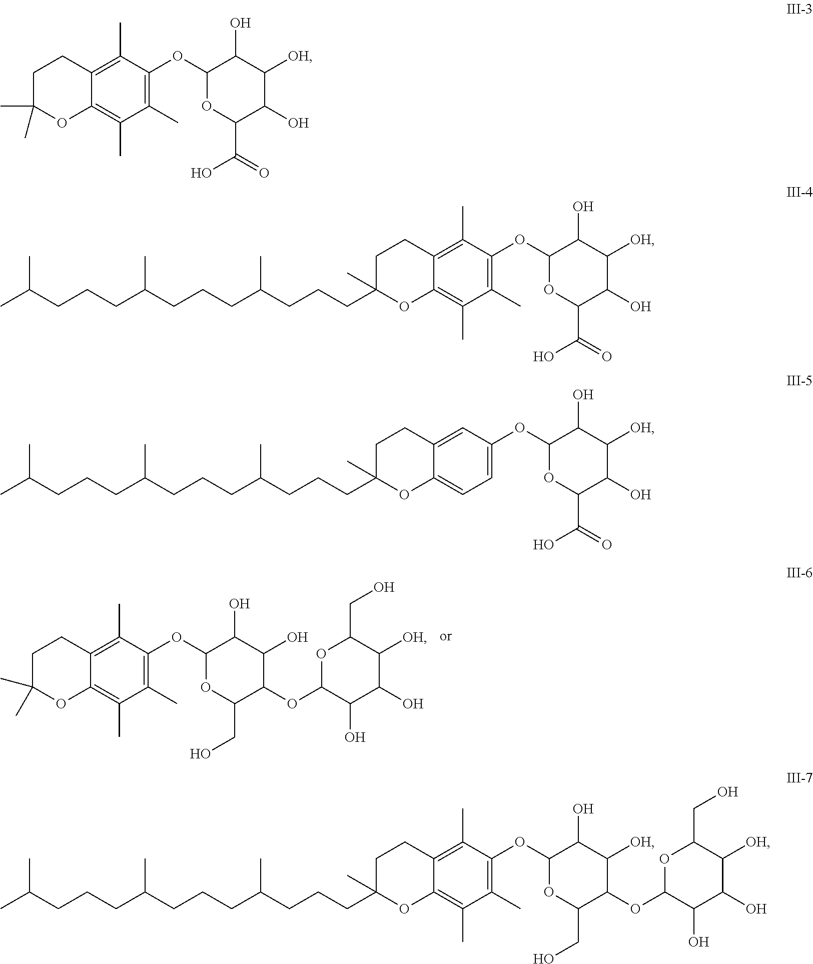 [SMILES: Cc1c(C)c2c(c(C)c1OC1OC(C(=O)O)C(O)C(O)C1O)CCC(C)(C)O2 ; Cc1c(C)c2c(c(C)c1OC1OC(C(=O)O)C(O)C(O)C1O)CCC(C)(CCCC(C)CCCC(C)CCCC(C)C)O2 ; CC(C)CCCC(C)CCCC(C)CCCC1(C)CCc2cc(OC3OC(C(=O)O)C(O)C(O)C3O)ccc2O1 ; Cc1c(C)c2c(c(C)c1OC1OC(CO)C(OC3OC(CO)C(O)C(O)C3O)C(O)C1O)CCC(C)(C)O2 ; Cc1c(C)c2c(c(C)c1OC1OC(CO)C(OC3OC(CO)C(O)C(O)C3O)C(O)C1O)CCC(C)(CCCC(C)CCCC(C)CCCC(C)C)O2]

19. A compound having a structure according to Formula V 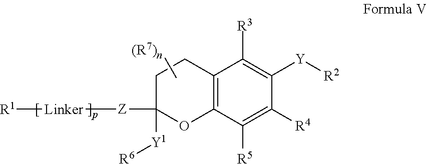
wherein:
R¹ is a hydrophilic group;
R² is a lipophilic group;
each or R³, R⁴ and R⁵ independently is H, C₁₋₆alkyl, —OC₁₋₆alkyl, C₃₋₆cycloalkyl, or —OC₃₋₆cycloalkyl;
R⁶ is H or C₁₋₃₀alkyl;
R⁷ is C₁₋₆alkyl;
n is 0, 1, 2, 3, or 4;
Y is a bond, O, or N(R^{a});
Y¹ is a bond, O, or N(R^{a});
Z is O, N(R^{a}), C(═O), (C═O)N(R^{a}), C(═O)O, N(R^{a})C(═O), OC(═O), or a bond;
Linker is —(CH₂)ₓO—, —(CH₂)ₓC(═O)O—, —(CH₂)ₓC(═O)N—, —(CH₂)ₓOC(═O)—, or —(CH₂)ₓNC(═O)—;
x is 1, 2, 3, or 4;
p is 0 or 1; and
each R^{a} independently is H, C₁₋₄alkyl.

20. A composition, comprising a compound according to claim 1.
